(19) United States
(12) Patent Application Publication
(10) Pub. No.: US 2023/0203055 A1
(43) Pub. Date: Jun. 29, 2023

(54) FUSED RING COMPOUND AND APPLICATION THEREOF IN MEDICINE

(71) Applicant: Betta Pharmaceuticals Co., LTD, Hangzhou (CN)

(72) Inventors: Hao Wu, Hangzhou (CN); Xiaoping Chen, Hangzhou (CN); Jun Yu, Hangzhou (CN); Yuan Lu, Hangzhou (CN); Jiangqi He, Hangzhou (CN); Wei Wang, Hangzhou (CN); Bo Zhan, Hangzhou (CN); Boyan Li, Hangzhou (CN); Yunlai Zhang, Hangzhou (CN); Dong Wang, Hangzhou (CN); Xiujun Xie, Hangzhou (CN); Xiaoguan Zhu, Hangzhou, (CN); Hong Lan, Beijing (CN); Jiabing Wang, Beijing (CN); Lieming Ding, Hangzhou (CN)

(21) Appl. No.: 17/997,328

(22) PCT Filed: Apr. 27, 2021
(86) PCT No.: PCT/CN2021/090082

§ 371 Date: Oct. 27, 2022

(30) Foreign Application Priority Data
Apr. 28, 2020 (CN) PCTCN/2020/087465
Aug. 20, 2020 (CN) 202010844659.0

(51) Int. Cl. C07D 491/052; C07D 491/044; C07D 491/147; C07D 519/00; C07D 487/04; C07D 498/22; C07D 471/04

(52) U.S. Cl. CPC C07D 491/052; C07D 471/04; C07D 487/04; C07D 491/044; C07D 491/147; C07D 498/22; C07D 519/00; C07B 2200/05

(57) ABSTRACT

The present invention relates to a novel compound (formula I), which has cancer treatment activity. The present invention also relates to a preparation method for the compound and a pharmaceutical composition containing the compound.

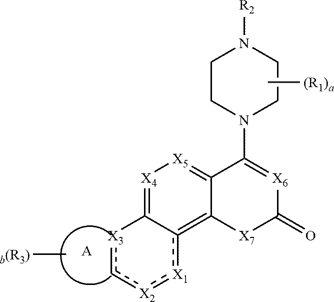

### TECHNICAL FIELD

The present invention relates to a novel fused-ring compound having cancer therapeutic activity. The present invention also relates to methods for the preparation of these compounds and pharmaceutical compositions containing them.

### BACKGROUND TECHNIQUE

Clinical data show that RAS is the gene with the highest mutation rate in human tumors, about 20-30% of all tumors have RAS mutations, about 98% of pancreatic cancer, 52% of colon cancer, 43% of multiple myeloma, and 32% of lung adenocarcinomas have RAS gene mutations. The most common mutation of RAS is point mutation, which often occurs at codons 12, 13, and 61, of which the 12th codon mutation is the most common. KRAS-G12C mutations account for approximately 10-20% of KRAS mutations and 14% in non-small cell lung cancer. RAS protein is a low-molecular-weight guanosine triphosphate (GTP)-binding protein with only one polypeptide chain, including two conformations: an active GTP-binding conformation and an inactive GDP-binding conformation. Under certain conditions, they can be transformed into each other to form the RAS cycle and regulate the activation of multiple downstream signaling pathways, the most important of which include the RAF-MEK-ERK and PI3K-AKT-mTOR signaling pathways. RAS is known as a “molecular switch” in the transmission of cellular signaling networks. Under normal circumstances, RAS is in an inactive state bound to GDP, but RAS is activated after receiving upstream signal stimulation, and the signal chain is only temporarily active. However, when RAS is mutated, the exchange frequency between RAS and GDP/GTP is accelerated, and RAS can combine with GTP for a long time, so that RAS and downstream signals are activated for a long time, and cell proliferation is out of control, resulting in malignant transformation of cells.

At present, drug development targeting KRAS mutations is one of the hotspots in current new drug research. KRAS G12C inhibitors AMG510 (WO2018217651A1) and MRTX849 (WO2019099524A1) have entered late clinical stages. The present invention provides a KRAS regulator with a novel structure.

### SUMMARY

The present invention provides compounds represented by general Formula (I), its tautomer, deuterated compound or pharmaceutically acceptable salt:

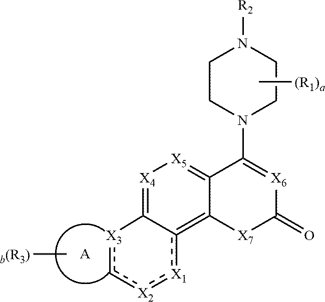

wherein,
R₁ is independently H, amino, halogen, C₁₋₃ cyano, C₁₋₃ hydroxyalkyl, C₁₋₆ alkyl, C₁₋₆ haloalkyl, C₁₋₆ alkoxy or C₁₋₆ haloalkoxy, or 2 R₁ and the connected atoms together form C₃₋₆ cycloalkyl or 3-6 membered heterocyclyl;
R₂ is acryloyl or substituted acryloyl;
A is selected from the group consisting of C₃₋₁₀ cycloalkyl, 3-10 membered heterocyclyl, C₆₋₁₂ aryl and 5-12 membered heteroaryl;
X₁ is independently —C(R₄)₁₋₂—(CH₂)₀₋₂—, —NR₄—(CH₂)₀₋₂—, —O—(CH₂)₀₋₂—, —S—(CH₂)₀₋₂—,

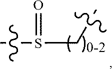

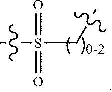

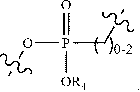

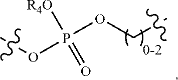

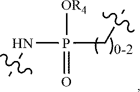

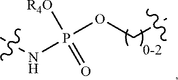

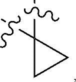

—CH═CH— or —N═CH—;
X₂ is independently —C(R₅)₁₋₂—(CH₂)₀₋₂—, —NR₅—(CH₂)₀₋₂—, —O—(CH₂)₀₋₂—, —S—(CH₂)₀₋₂—,

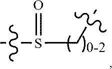

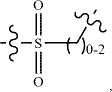

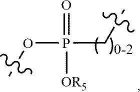

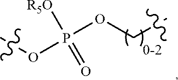

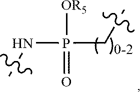

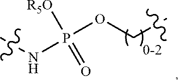

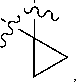

—CH═CH— or —N═CH—;
X₃ is C, CH or N;
X₄ is CR₆ or N;
X₅ is CR₇ or N;
X₆ is CR₈ or N;
X₇ is NR₉ or CHR₉;
R₆ and R₈ are independently selected from the group consisting of H, halogen, C₁₋₃ alkyl, C₁₋₃ haloalkyl, C₁₋₃ alkoxy, C₃₋₄ cycloalkyl, C₃₋₁₂ heterocycloalkyl, C₂₋₃ alkenyl, C₂₋₃ alkynyl, aryl and heteroaryl;
R₇ is independently H, hydroxy, C₁₋₆ alkyl, C₁₋₆ haloalkyl, C₁₋₆ alkoxy, -NH-C₁₋₆ alkyl, -N(C₁₋₆ alkyl)₂, cyano or halogen;
R₉ is independently C_{1-6,} alkyl, —C₀₋₃ alkylene-C₆₋₁₄ aryl, —C₀₋₃ alkylene-(5-14-membered heteroaryl), —C₀₋₃ alkylene-C₃₋₁₄ cycloalkyl, —C₀₋₃ alkylene-(3-14 membered heterocycloalkyl), —O—C₀₋₃ alkylene-C₆₋₁₄ aryl, —O—C₀₋₃ alkylene -(5-14 membered heteroaryl), —O—C₀₋₃ alkylene-C₃₋₁₄ cycloalkyl, -O—C₀₋₃ alkylene-(3-14 membered heterocycloalkyl), -NH-C₁₋₆ alkyl, -N (C₁₋₆ alkyl)₂, -NH—C₀₋₃ alkylcne-C₆₋₁₄ aryl, -NH—C₀₋₃ alkylene-(5-14 membered heteroaryl), -NH—C₀₋₃ alkylene-C₃₋₁₄ cycloalkyl or -NH—C₀₋₃ alkylene-(3-14 membered heterocycloalkyl), where said R₉ is optionally unsubstituted or further substituted by one or more R₉ₐ substituents;
R₃, R₄, R₅, and R₉ₐ are each independently selected from the group consisting of H, D, oxo, nitro, halogen, C₁₋₆ alkyl, C₁₋₆ cyano, C₁₋₆ haloalkyl, —C₀₋₃ alkylene-ORₐ, —C₀₋₃ alkylene-N ( Rₐ)₂, —C₀₋₃ alkylene—NRₐC(═O)Rₐ —C₀₋₃ alkylene—NR_{aC}(═O)ORₐ, —C₀₋₃ alkylene—NRₐS(═O)Rₐ, —C₀₋₃ alkylene—S(═O)Rₐ, —C₀₋₃ alkylene—S(═O)₂Rₐ, —C₀₋₃ alkylene—S(═O)₂N(Rₐ)₂, —C₀₋₃ alkylene-SRₐ, -C(₀₋₃ alkylene-S(Rₐ)₅, —C₀₋₃ alkylene—C(═O)N(Rₐ)₂, —C₀₋₃ alkylene-C(=O)R_{a,} —C₀₋₃ alkylenc-C(=O)OR_{a,} C₂₋₆ alkenyl, C₂₋₆ alkynyl, —C₀₋₃ alkylene-C₃₋₁₄ cycloalkyl, —C₀₋₃ alkylene-(3-14 membered heterocycloalkyl), —C₀₋₃ alkylene-C₆₋₁₄ aryl and —C₀₋₃ alkylene-(5-14 membered heteroaryl), where said —C₀₋₃ alkylene-C₃₋₁₄ cycloalkyl. —C₀₋₃ alkylene-(3-14 membered heterocycloalkyl), —C₀₋₃ alkylene-C₆₋₁₄ aryl or —C₀₋₃ alkylene-(5-14 membered heteroaryl) is optionally unsubstituted or further substituted with one or more Rₐ substituents, and each Rₐ is independently selected from the group consisting of H, halogen, C₁₋₆ alkyl, C₁₋₆ haloalkyl, C₃₋₁₄ cycloalkyl, 3-14 membered heterocycloalkyl, C₂₋₃ alkenyl, C₂₋₃ alkynyl, aryl and heteroaryl;
a and b are each independently selected from the group consisting of 1, 2, 3 and 4;

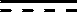

is a double bond or a single bond.

In certain embodiments, the compounds of Formula (I) is represented by formula (1-1),

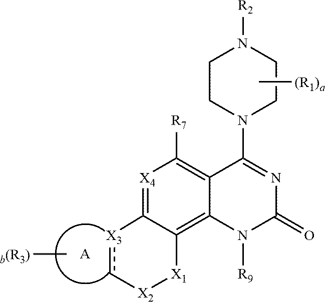

wherein,
R₁ is independently H, amino, halogen, C₁₋₃ cyano, C₁₋₃ hydroxyalkyl or C₁₋₆ alkyl, or 2 R₁ together with the attached atoms form C₃₋₆ cycloalkyl or 3- 6-membered heterocyclic group;
R₂ is acryloyl or substituted acryloyl;
ring A is selected from the group consisting of C₃₋₁₀ cycloalkyl, 3-10 membered heterocyclyl, C₆₋₁₂ aryl and 5-12 membered heteroaryl;
X₁ is independently —C(R₄)₂—(CH₂)₀₋₂—, —NR₄—(CH₂)₀₋₂—, —O—(CH₂)₀₋₂—, —S—(CH₂)₀₋₂—,

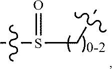

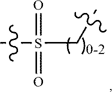

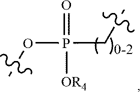

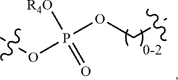

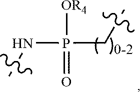

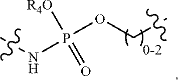

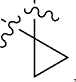

-C(=O)-(CH₂)₀₋₂-, —CH═CH— or —N═CH—, where said R₄ is independently selected from the group consisting of H, D, halogen and C₁₋₃ alkyl;
X₂ is independently C(R₅)₂—(CH₂)₀₋₂—, —NR₅—(CH₂)₀₋₂—, —O—(CH₂)₀₋₂—, —S—(CH₂)₀₋₂—,

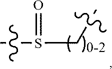

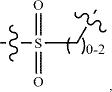

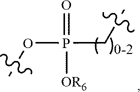

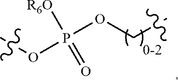

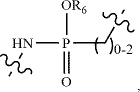

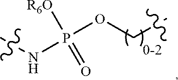

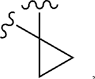

—C(═O)—(CH₂)₀₋₂—, —CH═CH— or —N═CH—, where said R₅ is independently selected from the group consisting of H, D, halogen and C₁₋₃ alkyl;
X₃ is C, CH or N;
X₄ is CR₆, or N;
R₆ is independently H, halogen, C₁₋₃ alkyl, C₁₋₃ haloalkyl, C₁₋₃ alkoxy, C₃₋₄ cycloalkyl, C₃₋₁₂ heterocycloalkyl, C₂₋₃ alkenyl, C₂₋₃ alkynyl, aryl or heteroaryl;
R₇ is independently H, hydroxy, C₁₋₆ alkyl. C₁₋₆ haloalkyl, C₁₋₆ alkoxy, -NH-C₁₋₆ alkyl, -N(C₁₋₆ alkyl)₂, cyano or halogen;
R₉ is independently —C₀₋₃ alkylene-C₆₋₁₄ aryl, —C₀₋₃ alkylene-(5-14-membered heteroaryl), —C₀₋₃ alkylene-C₃₋₁₄ cycloalkyl, —C₀₋₃ alkylene-(3-14 membered heterocycloalkyl), -O—C₀₋₃ alkylene-C₆₋₁₄ aryl, —O—C₀₋₃ alkylene-(5-14 membered heteroaryl), —O—C₀₋₃ alkylene-C₃₋₁₄ cycloalkyl, —O—C₀₋₃ alkylene-(3-14-membered heterocycloalkyl), —NH—C₁₋₆ alkyl, -N(C₁₋₆ alkyl)₂, —NH—C₀₋₃ alkylene-C₆₋₁₄ aryl, —NH-C₀₋₃ alkylene-(5-14-membered heteroaryl), -NH—C₀₋₃ alkylene-C₃₋₁₄ cycloalkyl or —NH-C₀₋₃ alkylene-(3-14-membered heterocycloalkyl), where said R₉ is optionally unsubstituted or further substituted by 1-4 R₉ₐ;
R₃ and R₉ₐ are each independently selected from the group consisting of H, D, oxo, nitro, halogen, C₁₋₆ alkyl, C₁₋₆ cyano, C₁₋₆ haloalkyl, —C₀₋₃ alkylene-ORₐ, —C₀₋₃ alkylene-N(Rₐ)₂ —C₀₋₃ alkylene—NRₐC(═O)Rₐ, —C₀₋₃ alkylene—NRₐC(═O)ORₐ, —C₀₋₃ alkylene-NRₐS(=O)₂R_{a,} —C₀₋₃ alkylene—S(═O)Rₐ, —C₀₋₃ alkylene—S(═O)₂Rₐ, —C₀₋₃ alkylene—S(═O)₂N(Rₐ)₂, —C₀₋₃ alkylene-SRₐ, —C₀₋₃ alkylene-S(Rₐ)₅, —C₀₋₃ alkylene—C(═O)N(R₁₄)₂, —C₀₋₃ alkylene-C(=O)R_{a,} —C₀₋₃ alkylene—C(═O)ORₐ, C₂₋₆ alkenyl, C₂₋₆ alkynyl, —C₀₋₃ alkylene-C₃₋₁₄ cycloalkyl, —C₀₋₃ alkylene-(3-14 membered heterocycloalkyl), —C₀₋₃ alkylene-C₆₋₁₄ aryl and —C₀₋₃ alkylene-(5-14-membered heteroaryl), where said —C₀₋₃ alkylene-C₃₋₁₄ cycloalkyl, —C₀₋₃ alkylene-(3-14-membered heterocycloalkyl), —C₀₋₃ alkylene —C₆₋₁₄ aryl or —C₀₋₃ alkylene-(5-14 membered heteroaryl) is optionally unsubstituted or further substituted with one or more Rₐ substituents, and each Rₐ is independently H , halogen, C₁₋₆ alkyl, C₁₋₆ haloalkyl, C₃₋₁₄ cycloalkyl, 3-14 membered heterocycloalkyl, C₂₋₃ alkenyl, C₂₋₃ alkynyl, aryl or heteroaryl;
a and b are independently 1, 2, 3 or 4;

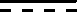

is a double bond or a single bond.

In certain embodiments,

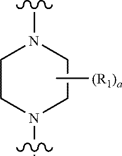

in Formula (I) is selected from the group consisting of

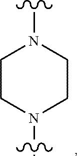

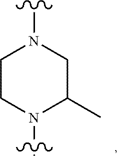

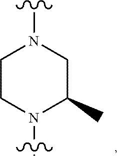

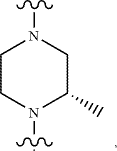

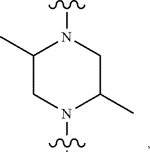

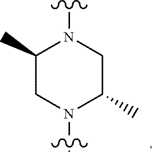

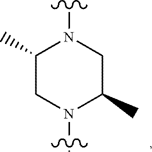

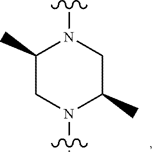

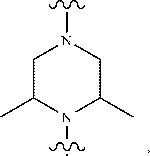

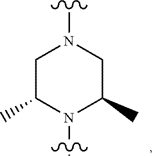

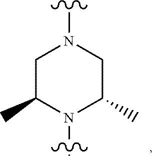

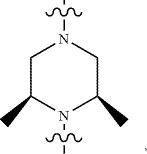

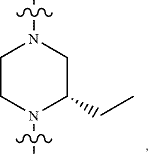

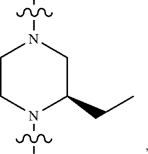

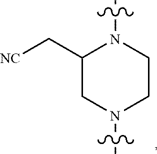

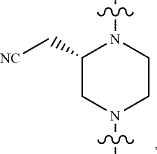

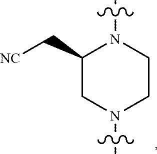

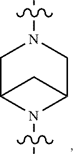

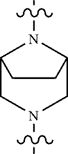

and

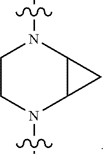

In certain embodiments, R² in Formula (I) is selected from the group consisting of

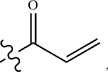

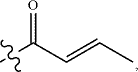

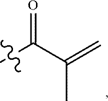

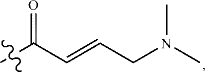

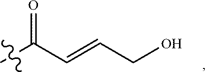

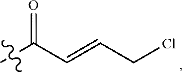

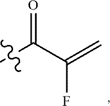

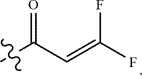

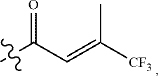

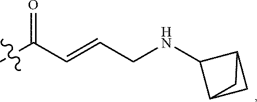

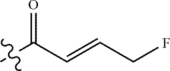

and

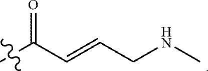

In certain embodiments, ring A in Formula (I) is selected from the group consisting of

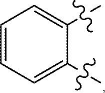

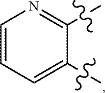

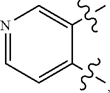

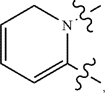

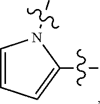

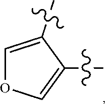

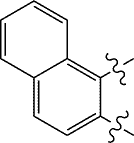

and

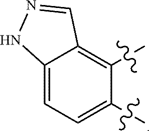

In certain embodiments, R₃ in Formula (I) is selected from the group consisting of H, halogen, oxo, —ORₐ, —C₀₋₃ alkylene-N(Rₐ)₂, and C₁₋₆haloalkyl, where said Rₐ is independently H, C₁₋₆ alkyl or C₁₋₆ haloalkyl.

In certain embodiments, X₁ in Formula (I) is selected from the group consisting of —C(R₄)₂—CH₂)₀₋₂—, —NH₁—(CH₂)₀₋₂—, —O—(CH₂)₀₋₂—, —C(O)—(CH₂)₀₋₂—,

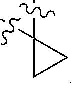

—CH═CH— and —N═CH—, where said R₄ is independently H, D, halogen or C₁₋₃ alkyl.

In certain embodiments, X₂ in Formula (I) is selected from the group consisting of —C(R₅)₂—(CH₂)₀₋₂—, —NH—(CH₂)₀₋₂—, —O—(CH₂)₀₋₂—, —C(O)—(CH₂)₀₋₂—,

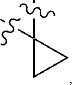

—CH═CH— and —N═CH—, where said R₅ is independently selected from the the group consisting of H, D, halogen and C₁₋₃ alkyl.

In certain embodiments, X₃ in Formula (I) is C, CH or N.

In certain embodiments, X₄ in Formula (I) is CR₆ or N, and the substituent R₆ is independently halogen, C₁₋₃ alkyl, C₁₋₃ haloalkyl or C₂₋₃ alkenyl.

In certain embodiments, X₅ in Formula (I) is CR₇ or N, and the substituent R₇ is independently H or C₁₋₃ alkyl.

In certain embodiments, X₆ in Formula (I) is CR₈ or N, and the substituent R₈ is independently H, halogen or C₁₋₃ alkyl, preferably X₆ is N.

In certain embodiments, X₇ in Formula (I) is NR₉ or CHR₉, preferably X₇ is NR₉.

In certain embodiments, R₉ in Formula (I) is selected from the group consisting of —C₀₋₃ alkylene-C₆₋₁₄ aryl, —C₀₋₃ alkylene-(5-14-membered heteroaryl), —C₀₋₃ alkylene--C₃₋₁₄ cycloalkyl, and —C₀₋₃ alkylene-(3-14 membered heterocycloalkyl), where said —C₀₋₃ alkylene-C₆₋₁₄ aryl, —C₀₋₃ alkylene-(5-14 membered heteroaryl), —C₀₋₃ alkylene-C₃₋₁₄ cycloalkyl or —C₀₋₃ alkylene-(3-14 membered heterocycloalkyl) is unsubstituted or further substituted by 1-4 substituents selected from the group consisting of halogen, C₁₋₃ alkyl, C₁₋₃ alkenyl, C₁₋₃ haloalkyl, —(CH₂)₀₋₃—S(Rₐ)₅, C₁₋₃ alkoxy, C₃₋₆ cycloalkyl, C₁₋₃ alkyl substituted C₃₋₆ cycloalkyl, —C₀₋₃ alkylene-phenyl and —C₀₋₃ alkylene-N(Rₐ)₂, and each Rₐ is independently H or C₁₋₆ alkyl; preferably R₉ is selected from the group consisting of

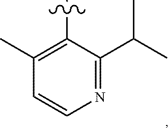

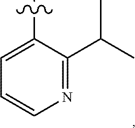

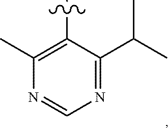

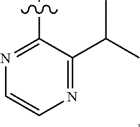

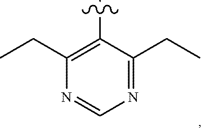

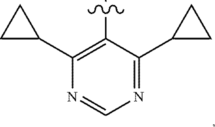

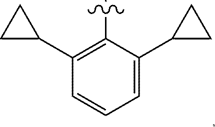

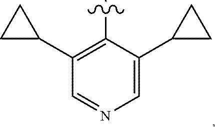

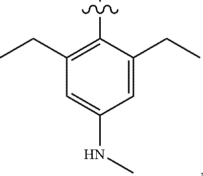

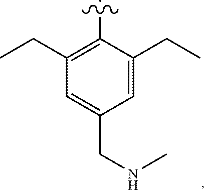

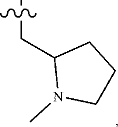

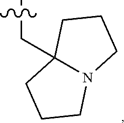

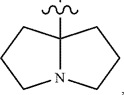

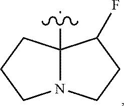

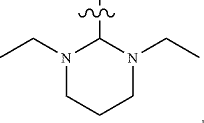

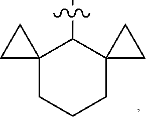

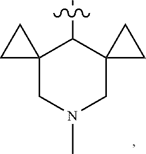

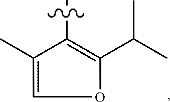

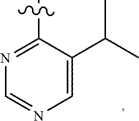

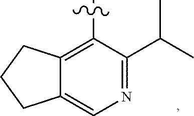

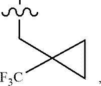

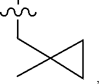

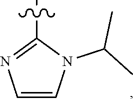

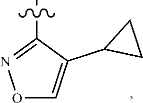

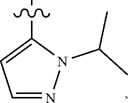

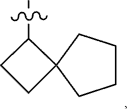

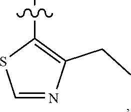

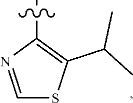

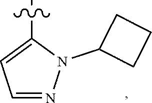

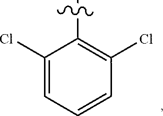

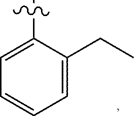

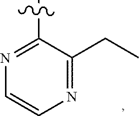

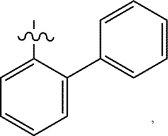

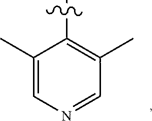

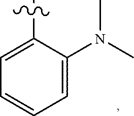

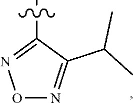

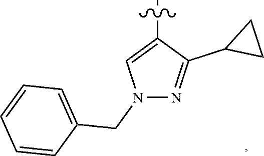

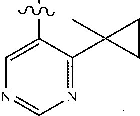

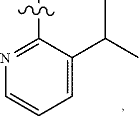

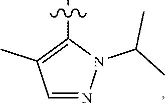

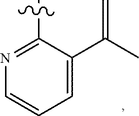

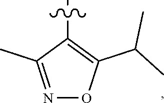

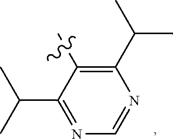

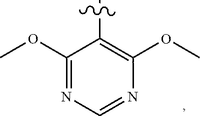

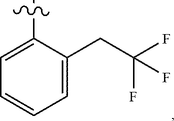

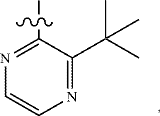

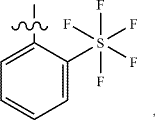

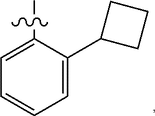

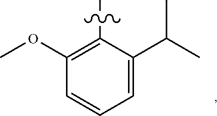

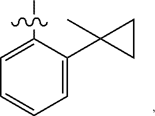

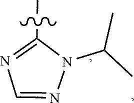

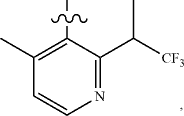

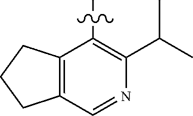

and

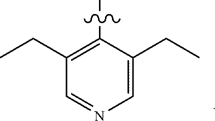

In certain embodiments,

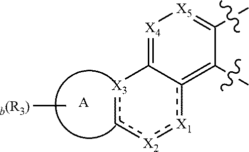

in formula (I) is selected from the group consisting of

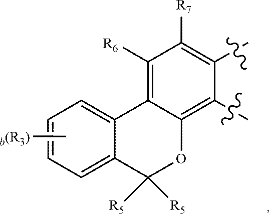

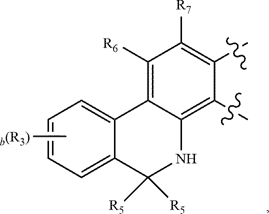

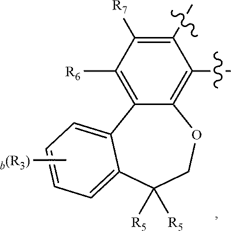

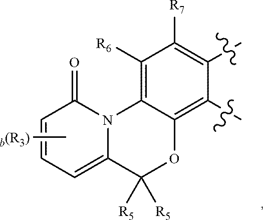

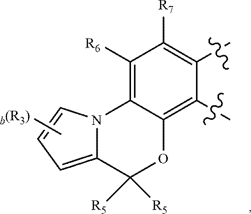

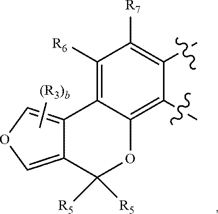

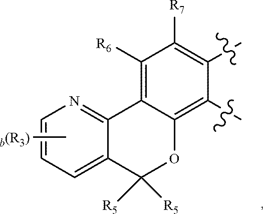

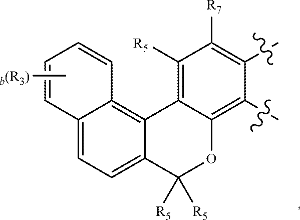

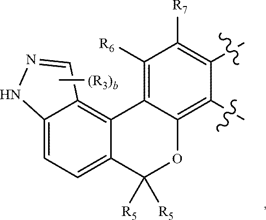

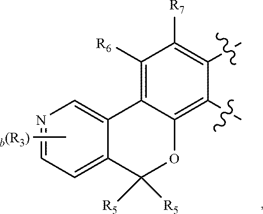

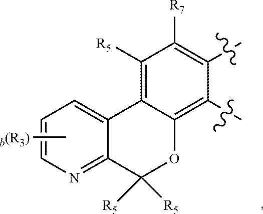

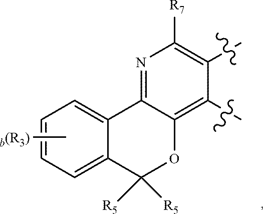

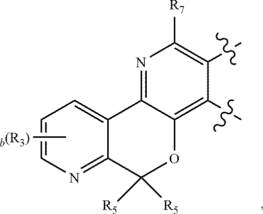

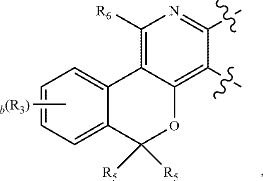

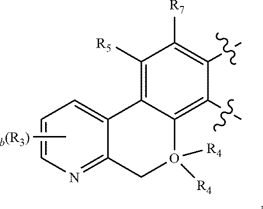

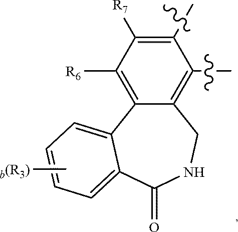

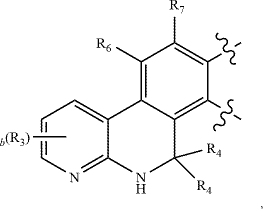

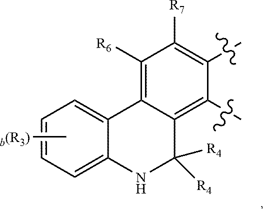

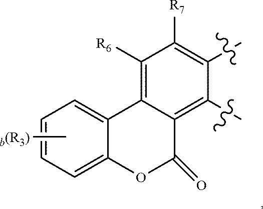

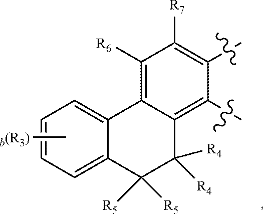

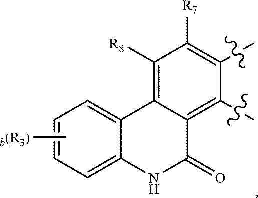

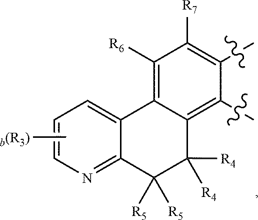

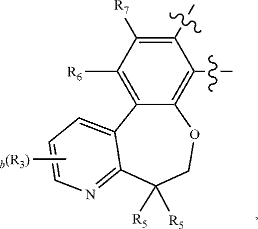

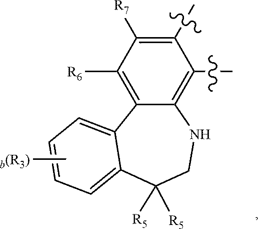

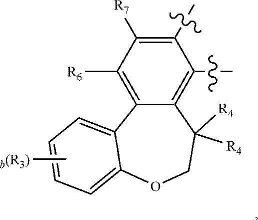

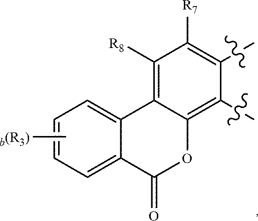

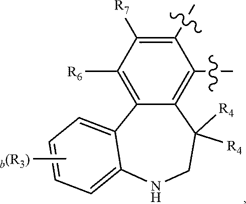

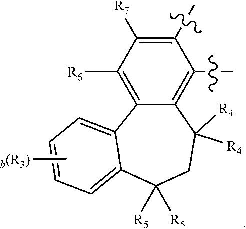

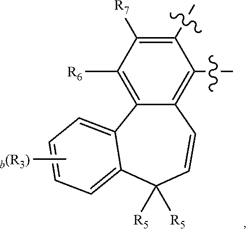

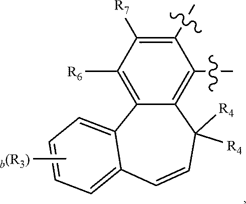

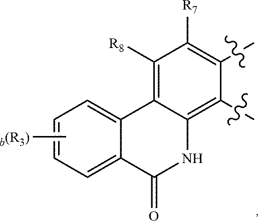

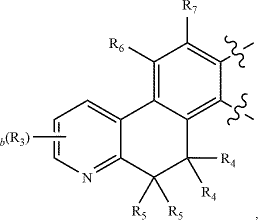

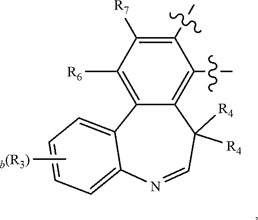

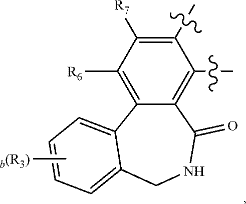

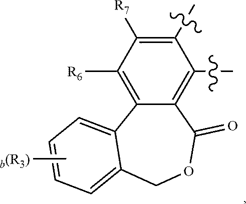

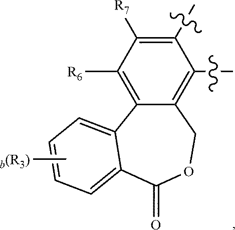

and

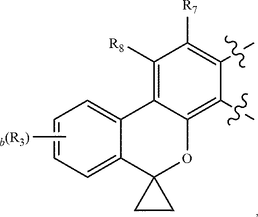

In certain embodiments, the Formula (I) is selected from Formula (Ia) to Formula (Ie),

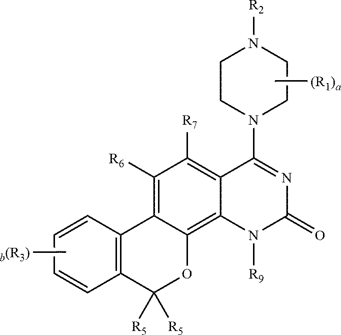

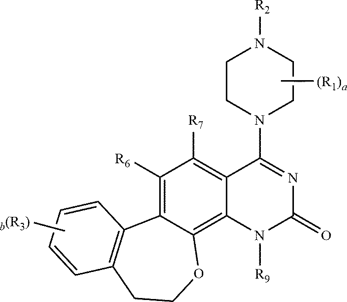

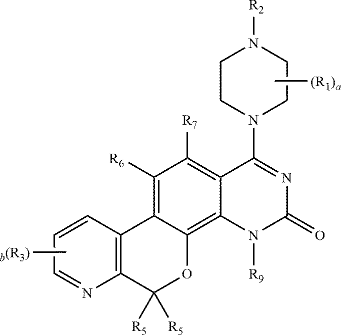

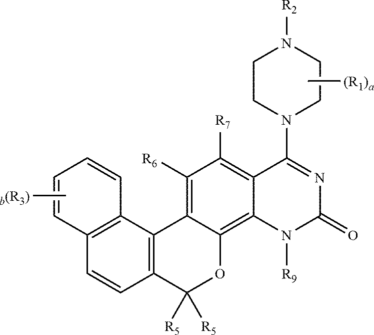

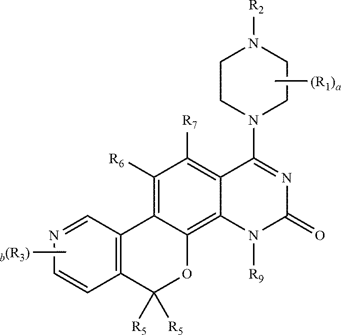

wherein, the substituents R₁-R₉, a and b are as defined in Formula (I).

The present invention also provides compounds represented by the general Formula (II), its tautomer, deuterated compound or pharmaceutically acceptable salt thereof,

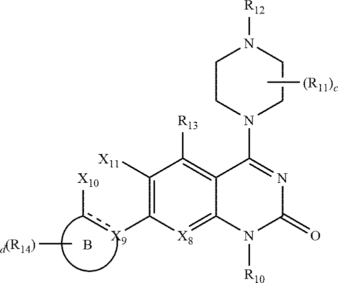

wherein, R₁₀ is selected from the group consisting of C₁₋₆ alkyl, —C₀₋₃ alkylene-C₆₋₁₄ aryl, —C₀₋₃ alkylene-(5-14-membered heteroaryl), —C₀₋₃ alkylenc-C₃₋₁₄ cycloalkyl, —C₀₋₃ alkylene-(3-14 membered heterocycloalkyl), —O—C₀₋₃ alkylene-C₆₋₁₄ aryl, —O—C₀₋₃ alkylene -(5-14 membered heteroaryl), —O—C₀₋₃ alkylene-C₃₋₁₄ cycloalkyl, —O—C₀₋₃ alkylene-(3-14 membered heterocycloalkyl), —NH—C₁₋₆ alkyl, -N(C₁₋₆ alkyl)₂, -NH—C₀₋₃ alkylene-C₆₋₁₄ aryl, —NH—C₀₋₃ alkylene-(5-14 membered Heteroaryl), -NH—C₀₋₃ alkylene-C₃₋₁₄ cycloalkyl, and —NH—C₀₋₃ alkylene-(3-14 membered heterocycloalkyl), where said R₁₀ is optionally unsubstituted or further substituted by 1-4 R₁₀ₐ substituents; preferably R₁₀ is selected from the group consisting of

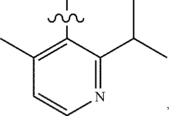

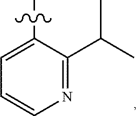

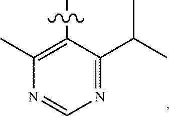

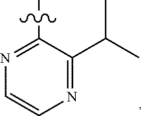

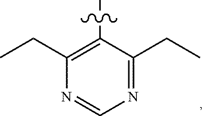

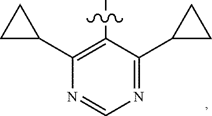

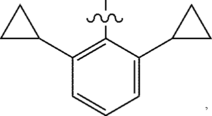

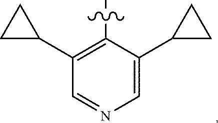

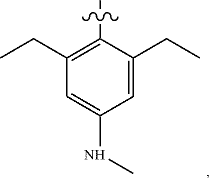

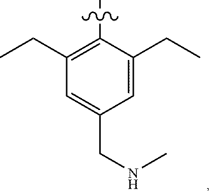

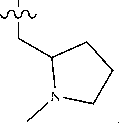

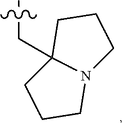

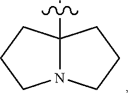

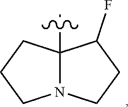

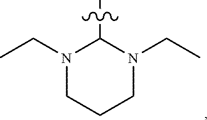

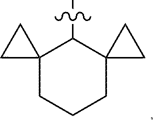

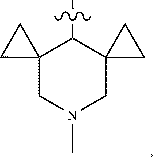

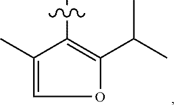

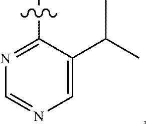

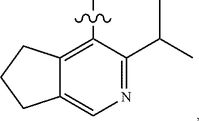

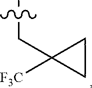

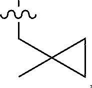

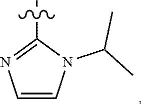

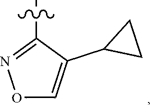

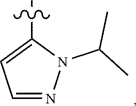

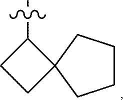

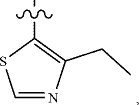

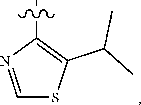

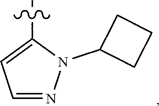

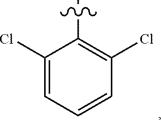

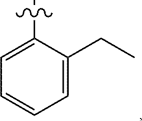

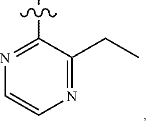

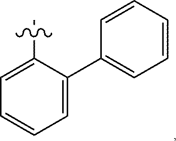

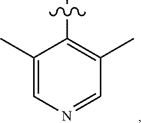

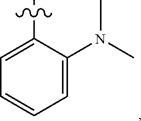

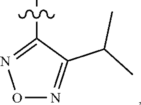

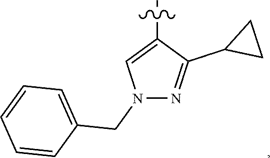

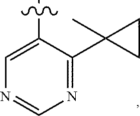

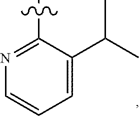

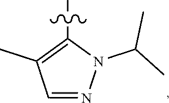

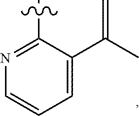

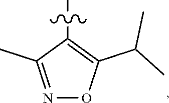

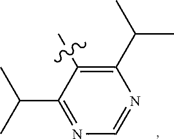

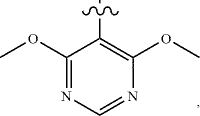

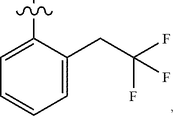

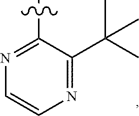

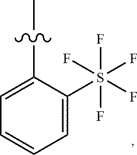

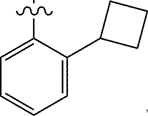

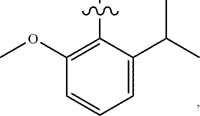

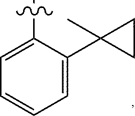

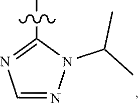

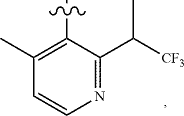

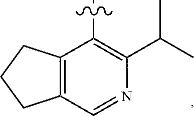

and

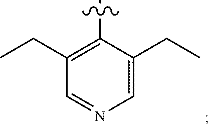

R₁₁ is independently H, amino, halogen, C₁₋₃ cyano, C₁₋₃ hydroxyalkyl or C₁₋₆ alkyl; preferably R₁₁ is methyl or ethyl cyano;
R₁₂ is acryloyl or substituted acryloyl; preferably R₁₂ is selected from the group consisting of

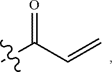

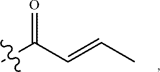

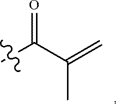

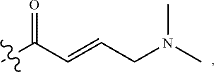

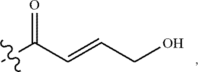

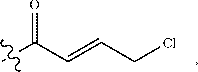

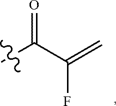

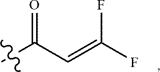

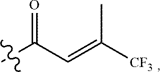

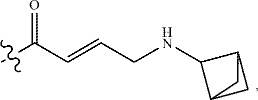

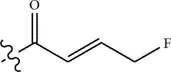

and

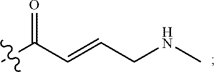

R₁₃ is independently H, hydroxy, C₁₋₆ alkyl, C₁₋₆ haloalkyl, C₁₋₆ alkoxy, -NH-C₁₋₆ alkyl, -N(C₁₋₆ alkyl)₂, cyano or halogen; preferably R₁₃ is H;
ring B is selected from the group consisting of C₃₋₁₀ cycloalkyl, 3-10 membered heterocyclyl, C₆₋₁₂ aryl and 5-12 membered heteroaryl;
X₈ is CR₁₅ or N; preferably X₈ is selected from the group consisting of N, halogen and CH;
X₉ is C, CH or N;
X₁₀ and X₁₁ are independently —O—(CH₂)₀₋₂— or CH₂;
R₁₄, R₁₅ and R₁₀ₐ are independently selected from the group consisting of hydrogen, oxo, acyl, cyano, halogen, C₁₋₆ alkyl, C₁₋₆ haloalkyl, —(CH₂)₀₋₃—OR_{b}, —(CH₂)₀₋₃ —N(R_{b})₂, —(CH₂)₀₋₃₋S(═O)R_{b}, —(CH₂)₀₋₃—S(═O)₂R_{b}, —(CH₂)₀₋₃—SR_{b}, —(CH₂ )₀₋₃—S(Rₐ)₅, —(CH₂)₀₋₃—C(═O)N(R_{b})₂, C₂₋₃ alkenyl, C₂₋₃ alkynyl, —C₀₋₃ alkylene-C₃₋₁₄ cycloalkyl, —C₀₋₃ alkylene-(3-14 membered heterocycloalkyl), —C₀₋₃ alkylene-C₆₋₁₄ aryl and —C₀₋₃ alkylene-(5 -14-membered heteroaryl), and each R_{b} is independently selected from the group consisting of H, C₁₋₆ alkyl, C₁₋₆ haloalkyl, C₃₋₁₄ cycloalkyl, 3-14 membered heterocycloalkyl, C₂₋₃ alkene , C₂₋₃ alkynyl, aryl and heteroaryl;
c and d are independently 1, 2, 3 or 4;
[symbol] is a double bond or a single bond.

In certain embodiments, the Formula (II) is selected from Formula (IIa) to Formula (IId):

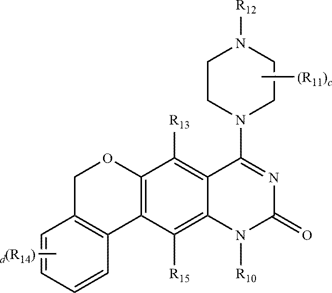

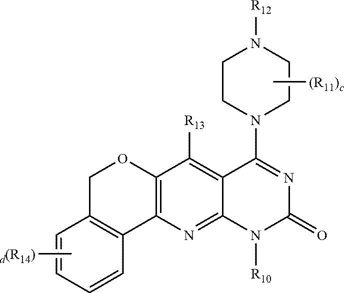

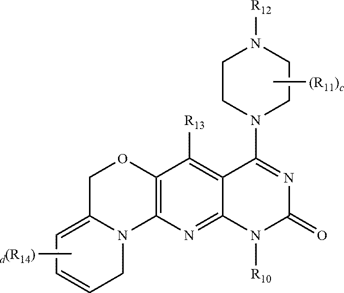

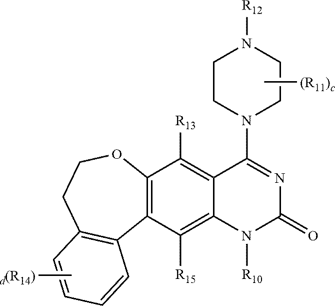

wherein, the substituents are as defined in Formula (II).

The present invention also provides a pharmaceutical composition, comprising a therapeutically effective amount of at least one compound represented by Formula (I) or Formula (II) and at least one pharmaceutically acceptable carrier.

The present invention further provides a pharmaceutical composition, wherein a weight ratio of the compound represented by Formula (I) or Formula (II) to the pharmaceutically acceptable carrier is 0.0001 :1-10.

The present invention provides the use of the compound represented by the structural Formula (I) or the Formula (II) or the pharmaceutical composition in the preparation of a medicine.

In certain embodiments, the method of preparing a compound of Formula (I) comprises the steps of:

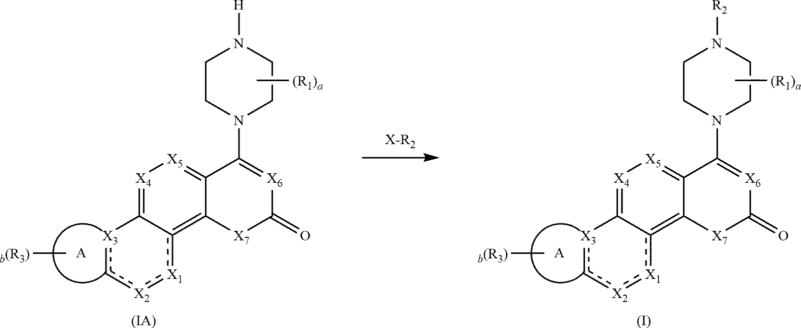

acylating the compound of Formula (IA) with a compound of X-R₂ under basic conditions, to obtain a compound of Formula (I), its tautomer, deuterated compound or pharmaceutically acceptable salt, wherein X is halogen; R₁-R_{3,} X₁-X₇, a or b are as defined in Formula (I).

The present invention further provides a preferred technical solution for the use of the compound represented by the formula (I) or the formula (II) or the pharmaceutical composition in the preparation of a medicine.

Preferably, the use is in the preparation of a drug for treating and/or preventing cancer.

Preferably, the use is in the preparation of a medicament for treating a disease mediated by KRAS G12C.

Preferably, the disease is cancer. Preferably, the cancer is selected from the group consisting of breast cancer, multiple myeloma, bladder cancer, endometrial cancer, gastric cancer, cervical cancer, rhabdomyosarcoma, non-small cell lung cancer, small cell lung cancer, pleomorphic lung cancer, ovarian cancer, esophagus cancer, melanoma, colorectal cancer, hepatocellular carcinoma, head and neck tumor, hepatobiliary cell carcinoma, myelodysplastic syndrome, malignant glioma, prostate cancer, thyroid cancer, xuwang’s cell tumor, lung squamous cell carcinoma, lichenoid keratosis, synovial sarcoma, skin cancer, pancreatic cancer, testicular cancer and liposarcoma.

The present invention also provides a method for treating and/or preventing diseases, comprising administering a therapeutically effective amount of at least any compound represented by structural Formula (I) or Formula (II) or a pharmaceutical composition containing the compound to a subject in need thereof.

The present invention also provides a method for treating and/or preventing a disease mediated by KRAS G12C, comprising administering a therapeutically effective amount of at least any compound represented by structural Formula (I) or Formula (II) or the pharmaceutical compositions comprising the same to a subject.

The present invention also provides a method for treating cancer, comprising administering a therapeutically effective amount of at least any compound represented by structural Formula (I) or Formula (II) or a pharmaceutical composition comprising the same to a subject in need thereof.

Preferably, in the above method, the KRAS G12C-mediated disease is cancer.

Preferably, in the above-mentioned method, the cancer is selected from the group consisting of breast cancer, multiple myeloma, bladder cancer, endometrial cancer, gastric cancer, cervical cancer, rhabdomyosarcoma, non-small cell lung cancer, small cell lung cancer, pleomorphic lung cancer, ovarian cancer, esophageal cancer, melanoma, colorectal cancer, hepatocellular tumor, head and neck tumor, hepatobiliary cell carcinoma, myelodysplastic syndrome, malignant glioma, prostate cancer, thyroid cancer, xuwang’s cell tumor, lung squamous cell carcinoma, lichenoid keratosis, synovial sarcoma, skin cancer, pancreatic cancer, testicular cancer and liposarcoma.

Unless otherwise specified, the general chemical terms used in the general structural Formula have their usual meanings.

For example, unless otherwise specified, the term “halogen” used in the present invention refers to fluorine, chlorine, bromine or iodine.

In the present invention, unless otherwise specified, “alkyl” will be understood to mean a linear or branched monovalent saturated hydrocarbon group. For example, alkyl includes methyl, ethyl, propyl, isopropyl, n-butyl, isobutyl, sec-butyl, tert-butyl, n-pentyl, 3-(2-methyl)butyl, 2-pentyl, 2-methylbutyl, neopentyl, n-hexyl, 2-hexyl, 2-methylpentyl, etc. Similarly, the “₁₋₈” in “C₁₋₈ alkyl” refers to a straight-chain or branched group containing 1, 2, 3, 4, 5, 6, 7 or 8 carbon atoms.

“C1-3 alkylene” refers to methylene, 1,2-ethylene, 1,3-propylene or 1,2-isopropylene.

“Alkoxy” refers to the oxyether form of the aforementioned linear or branched alkyl group, which is —O—alkyl.

In the present invention, “a”, “an”, “the”, “at least one” and “one or more” can be used interchangeably. Thus, for example, a composition comprising “a” pharmaceutically acceptable excipient can be interpreted to mean that the composition includes “one or more” pharmaceutically acceptable excipients.

The term “aryl” in the present invention, unless otherwise specified, will be understood to mean an unsubstituted or substituted monocyclic or condensed ring aromatic group including carbon ring atoms. In a further embodiment, the aryl group is a 6 to 10 membered monocyclic or bicyclic aromatic ring group. In a further embodiment, it is phenyl and naphthyl. Most preferred is phenyl. The aryl ring can be fused to a heteroaryl, heterocyclyl, or cycloalkyl, wherein the ring attached to the parent structure is an aryl ring, non-limiting examples including, but not limited to, benzocyclopentyl.

The term “heterocyclyl”, as used herein, unless otherwise specified, will be understood to mean an unsubstituted or substituted 3-8 membered stable monocyclic ring consisting of carbon atoms and 1-3 heteroatoms selected from N, O or S. Ring systems, which are saturated or partially unsaturated monocyclic or polycyclic cyclic hydrocarbon substituents, comprising 3 to 20 carbon atoms, wherein nitrogen or sulfur heteroatoms can be selectively oxidized, and nitrogen heteroatoms can be selectively ground is quatemized. The heterocyclyl can be attached to any heteroatom or carbon atom to form a stable structure. Examples of these heterocyclyls include, but are not limited to azetidinyl, pyrrolidinyl, piperidinyl, piperazinyl, oxopiperazinyl, oxopiperidinyl, tetrahydrofuranyl, dioxolane, tetrahydroimidazolyl, tetrahydrothiazolyl, tetrahydrooxazolyl, tetrahydropyranyl, morpholinyl, thiomorpholinyl, thiomorpholinyl sulfoxide, thiomorpholinylsulfone and tetrahydro oxadiazolyl.

The term “heteroaryl”, as used herein, unless otherwise specified, will be understood to mean an unsubstituted or substituted stable 5- or 6-membered monocyclic aromatic ring system or an unsubstituted or substituted 9- or 10-membered benzo-fused heteroaromatic ring system or bicyclic heteroaromatic ring system, which consists of carbon atoms and 1-4 heteroatoms selected from N, O or S, and wherein the nitrogen or sulfur heteroatoms can be selectively oxidized. The nitrogen heteroatoms can be selectively quaternized. The heteroaryl group can be attached to any heteroatom or carbon atom to form a stable structure. Examples of heteroaryl groups include, but are not limited to thienyl, furyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzothiazolyl, benzothiazolyl, benzene and thiadiazolyl, benzotriazolyl adenine, quinolinyl or isoquinolinyl. The heteroaryl group can be fused to an aryl, heterocyclyl or cycloalkyl ring, wherein, the ring connected with the parent structure is a heteroaryl ring.

The term “cycloalkyl” refers to a cyclic saturated or partially unsaturated monocyclic or polycyclic cyclic hydrocarbon substituent having 3 to 10 carbon atoms, for example, cyclopropyl, cyclobutyl, cyclopentyl or cyclohexyl .

The term “substituted” will be understood to mean that one or more hydrogen atoms in the group are replaced by the same or different substituents. Typical substituents include, but are not limited to halogen (F, Cl, Br or I), C₁₋₈ alkyl, C₃₋₁₂ cycloalkyl, —OR₁, —SR₁, ═O, ═S, —C(O)—R₁ , —C(S)R₁, ═NR₁, —C(O)OR₁, —C(S)OR₁, —NR₁R₂, —C(O)NR₁R₂, cyano, nitro, —S(O)₂R₁, —OS (O₂) OR₁, —OS(O)₂R₁, —OP(O)(OR₁)(OR₂). Wherein R₁ and R₂ are independently selected from —H, C₁₋₆ alkyl, and C₁₋₆ haloalkyl. In certain embodiments, the substituents are independently selected from the group comprising —F, —Cl, —Br, —I, —OH, trifluoromethoxy, ethoxy, propoxy, isopropoxy, n-butoxy group, isobutoxy, tert-butoxy, —SCH₃, —SC₂H₅, formaldehyde, —C(OCH₃), cyano, nitro, —CF₃, —OCF₃, amino, dimethylamino, methylthio, sulfonyl and acetyl groups.

Examples of substituted alkyl groups include, but are not limited to, 2,3-dihydroxypropyl, 2-aminoethyl, 2-hydroxyethyl, pentachloroethyl, trifluoromethyl, methoxymethyl, pentafluoroethyl , phenylmethyl, dioxenylmethyl and piperazinylmethyl.

Examples of substituted alkoxy groups include, but are not limited to, 2-hydroxyethoxy, 2-fluoroethoxy, 2,2-difluoroethoxy, 2-methoxyethoxy, 2-aminoethoxy, 2,3-dihydroxypropoxy, cyclopropylmethoxy, aminomethoxy, trifluoromethoxy, 2-diethylaminoethoxy, 2-ethoxycarbonylethoxy, 3- hydroxypropoxy.

The term “pharmaceutically acceptable salt” will be understood to mean a salt prepared from a pharmaceutically acceptable non-toxic base or acid. When the compound provided by the present invention is an acid, its corresponding salt can be conveniently prepared from pharmaceutically acceptable non-toxic bases, including inorganic bases and organic bases. Salts derived from inorganic bases include aluminum, ammonium, calcium, copper (high and low prices), ferric, ferrous, lithium, magnesium, manganese (high and low prices), potassium, sodium, zinc and the like. Particularly preferred are ammonium, calcium, magnesium, potassium and sodium salts. Pharmaceutically acceptable non-toxic organic bases that can be derivatized into salts include primary, secondary and tertiary amines, as well as cyclic amines and amines containing substituents, such as naturally occurring and synthetic amines containing substituents. Other pharmaceutically acceptable non-toxic organic bases capable of forming salts, including ion exchange resins and arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, reduced glucosamine, glucosamine, histidine, haramine, isopropylamine , lysine, methyl glucamine, morpholine, piperazine, piperidine, polyamine resin, procaine, purine, theobromine, triethylamine, trimethylamine, tripropylamine, tromethamine, etc.

When the compound provided by the present invention is a base, the corresponding salt can be conveniently prepared from pharmaceutically acceptable non-toxic acids, including inorganic acids and organic acids. Such acids include, for example, acetic acid, benzenesulfonic acid, benzoic acid, camphorsulfonic acid, citric acid, ethanesulfonic acid, isethionic acid, formic acid, fumaric acid, gluconic acid, glutamic acid, hydrobromic acid, Hydroiodic acid, perchloric acid, hydrochloric acid, isethionic acid, propionic acid, glycolic acid, lactic acid, maleic acid, malic acid, mandelic acid, methanesulfonic acid, mucic acid, nitric acid, oxalic acid, hexanoic acid, pantothenic acid, phosphoric acid , succinic acid, sulfuric acid, 2-naphthalenesulfonic acid, cyclohexylamine sulfonic acid, salicylic acid, saccharic acid, trifluoroacetic acid, tartaric acid and p-toluenesulfonic acid. Preferably, citric acid, hydrobromic acid, formic acid, hydrochloric acid, maleic acid, phosphoric acid, sulfuric acid and tartaric acid. More preferably, formic acid and hydrochloric acid.

Since the compound represented by Formula (I) or Formula (II) will be used as a medicine, it is preferable to use a certain purity, for example, at least 60% purity, more suitable purity is at least 75%, and particularly suitable purity is at least 98% (% is weight ratio).

The prodrug of the compound of the present invention is included in the protection scope of the present invention. Generally, the prodrug refers to a functional derivative that is easily converted into a desired compound in the body. For example, any pharmaceutically acceptable salt, ester, salt of ester or other derivative of the compound of the present application can directly or indirectly provide the compound of the present application or its pharmaceutically active metabolite or residues.

The compound of the present invention may contain one or more asymmetric centers, and may produce diastereomers and optical isomers from this. The present invention includes all possible diastereomers and their racemic mixtures, their substantially pure resolved enantiomers, all possible geometric isomers and their pharmaceutically acceptable salts.

When compounds of Formula (I) or Formula (II) are substituted with heavier isotopes such as deuterium may offer certain therapeutic advantages, this is due to greater metabolic stability, such as increased in vivo half-life or reduced dosage requirements.

When the compound represented by Formula (I) or Formula (II) and its pharmaceutically acceptable salt have solvates or polymorphs, the present invention includes any possible solvates and polymorphs. The type of solvent that forms the solvate is not particularly limited, as long as the solvent is pharmaceutically acceptable. For example, water, ethanol, propanol, acetone and similar solvents can be used.

The term “composition”, as used herein, will be understood to mean a product comprising a specified amount of each specified ingredient, and any product produced directly or indirectly from a combination of specified amounts of each specified ingredient. Therefore, pharmaceutical compositions containing the compounds of the present invention as active ingredients and methods for preparing the compounds of the present invention are also part of the present invention. In addition, some crystalline forms of the compound may exist in polymorphs, and this polymorph is included in the present invention. In addition, some compounds can form solvates with water (ie, hydrates) or common organic solvents, and such solvates also fall within the scope of the present invention.

The pharmaceutical composition provided by the present invention includes as an active component a compound represented by Formula (I) or Formula (II) (or a pharmaceutically acceptable salt thereof), a pharmaceutically acceptable excipient and other optional therapeutic components or accessories. Although in any given case, the most suitable way of administering the active ingredient depends on the particular subject to be administered, the nature of the subject and the severity of the disease, the pharmaceutical composition of the present invention includes oral, rectal, topical and a pharmaceutical composition for parenteral administration (including subcutaneous administration, intramuscular injection, and intravenous administration). The pharmaceutical composition of the present invention can be conveniently prepared in a unit dosage form known in the art and prepared by any preparation method known in the pharmaceutical field.

In general, to treat the conditions or discomforts shown above, the dose level of the drug is about 0.01 mg/kg body weight to 150 mg/kg body weight per day, or 0.5 mg to 7 g per patient per day. For example, inflammation, cancer, psoriasis, allergies/asthma, diseases and discomforts of the immune system, diseases and discomforts of the central nervous system (CNS), the effective treatment drug dosage level is 0.01 mg/kg body weight to 50 mg/kg body weight per day, or 0.5 mg to 3.5 g per patient per day.

However, it is understood that lower or higher dosages than those mentioned above may be required. The specific dosage level and treatment plan for any particular patient will depend on many factors, including the activity of the specific compound used, age, weight, overall health, gender, diet, administration time, administration route, excretion rate, and the condition of drug combination and the severity of the specific disease being treated.

### Synthetic Scheme

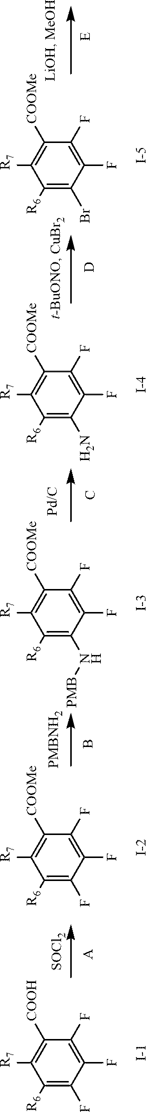

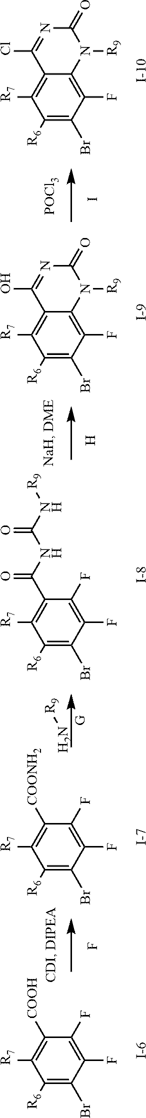

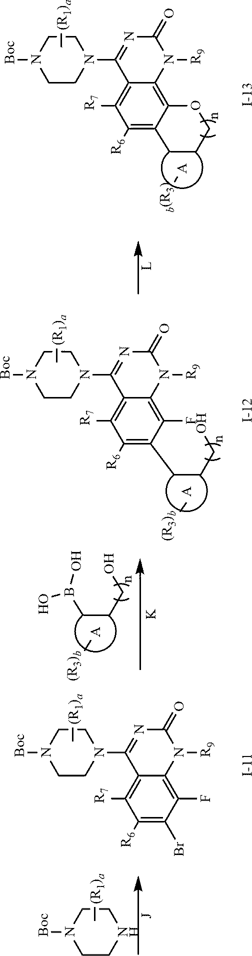

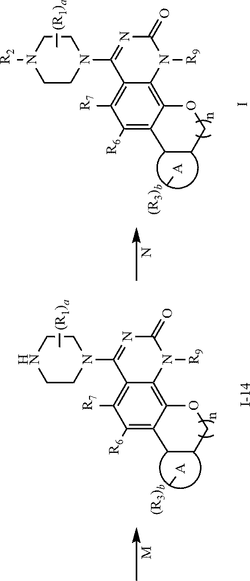

Step A: Compound I-1 is methylated to obtain compound I-2;
Step B: Compound I-2 is subjected to substitution reaction with PMBNH₂ to obtain compound I-3;
Step C: Compound I-3 removes the PMB protecting group under the action of Pd/C;
Step D: Compound I-4 converts the amino group into bromine under the action of CuBr₂;
Step E: Compound I-5 is hydrolyzed under alkaline conditions such as LiOH to obtain compound I-6;
Step F: Compound I-6 converts the carboxyl group into an amide under the action of CDI;
Step G: Compound I-7 introduces R₉ group under the action of oxalyl chloride;
Step H: Compound I-8 is self-ring closed under the action of strong base such as NaH to obtain compound I-9;
Step 1: Phosphorus oxychloride is stirred at high temperature to convert —OH of I-9 into —Cl to obtain compound I-10;
Step J: under basic conditions, such as DIEA etc, compound I-10 and

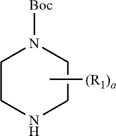

.undergo substitution reaction to compound I-11;
Step K: Under the Pd catalyst conditions, such as Pd(dppf)Cl₂, compound I -11 and

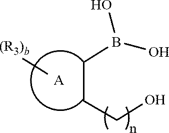

are coupled through Suzuki to introduce A ring to obtain compound I-12, and n is selected from 1, 2, 3 or 4;
Step L: under alkaline conditions, such as under the action of Cs₂CO_{3,} compound I-12 itself is closed;
Step M: under acidic conditions, such as trifluoroacetic acid, etc., remove the Boc protecting group;
Step N: Under weak basic conditions, such as sodium bicarbonate, etc., the target compound I can be obtained by introducing the R₂ group.

Synthetic route 2

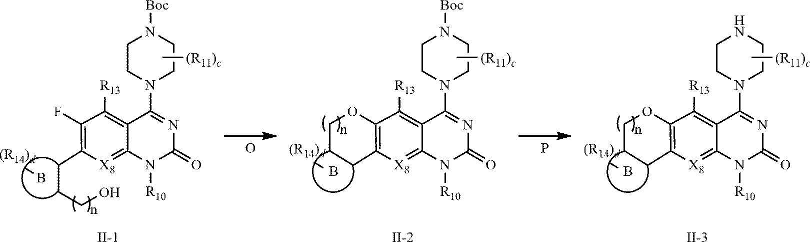

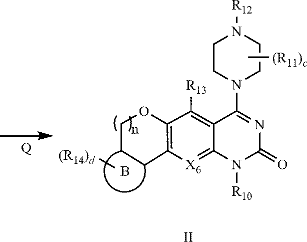

Step O: Synthesize compound II-1 through a similar procedure to compound I-12, and compound II-1 is self-ring closed under alkaline conditions such as Cs₂CO₃;
Step P: under acidic conditions such as trifluoroacetic acid, etc., remove the Boc protecting group;
Step Q: Under weak basic conditions such as sodium bicarbonate, etc., the target compound I can be obtained by introducing the R₁₂ group.

### EXAMPLES

In order to make the above content clearer and clearer, the present invention will use the following embodiments to further illustrate the technical solution of the present invention. The following examples are only used to illustrate specific implementations of the present invention, so that those skilled in the art can understand the present invention, but are not used to limit the protection scope of the present invention. In the specific embodiments of the present invention, technical means or methods that are not specifically described are conventional technical means or methods in the art.

Unless otherwise specified, all parts and percentages in the present invention are calculated by weight, and all temperatures refer to ℃.

The following abbreviations have been used:
CDI: carbonyldiimidazole;
DCM: dichloromethane;
DIPEA: diisopropylethylamine;
DME: dimethyl ether;
DMF: N,N-dimethylformamide;
DMSO: dimethyl sulfoxide;
EA: ethyl acetate;
MeOH: methanol;
PE: petroleum ether;
Pd/C: Palladium on Carbon;
THF: tetrahydrofuran:
TFA: trifluoroacetic acid;
pre-TLC: preparation of thin layer chromatography silica gel plate;
pre-HPLC: Preparative high performance liquid chromatography;
PMBNH₂: 4-methoxybenzylamine;
SOCl₂: thionyl chloride;
CDI: N,N′-Carbonyldiimidazole.

Synthesis of Intermediate M-11

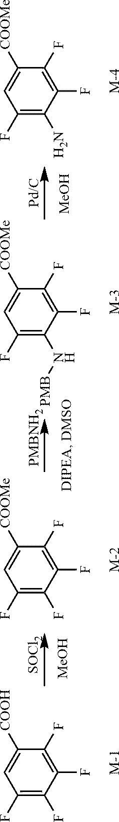

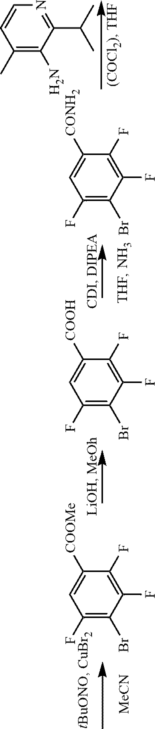

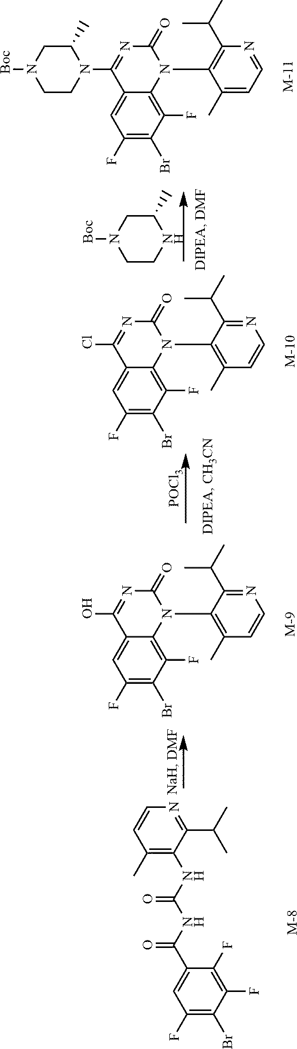

### Step 1: Synthesis of Compound M-2

The commercially available raw material M-1 (11.64 g, 60 mmol) was dissolved in methanol (80 mL), and then thionyl chloride (21.41 g, 180.00 mmol, 13.05 mL) was added dropwise, after dropping, the reaction was incubated at 55° C. overnight, TLC confirmed complete conversion of starting material. Concentrate to remove methanol. 120 mL of ethyl acetate was added, washed with 40 mL of saturated aqueous sodium chloride solution for 2 times, and the solution was separated. Add anhydrous sodium sulfate to dry. Filtration, removal of desiccant, and concentration to constant weight, obtained a colorless liquid M-2 (10.1 g, 48.53 mmol, 81% yield).

### Step 2: Synthesis of Compound M-3

Compound M-2 (10.1 g, 48.53 mmol) and 4-methoxybenzylamine (9.99 g, 72.80 mmol) were dissolved in DMSO (50 mL), diisopropylethylamine (25.09 g, 194.13 mmol, 33.81 mL) was added, heated to 60° C., and stirred the reaction. The reaction was stirred for 3.5 hours, and LC-MS detection showed that the conversion of the starting material was complete. After the reaction mixture was cooled to room temperature, it was added to 250 g of an ice-water mixture and stirred for 10 minutes. Add 200 mL×2 of ethyl acetate for extraction and separation; combine the organic phases. Saturated aqueous sodium chloride solution 120 mL was added for 2 times, washed and separated. Dry with anhydrous sodium sulfate, remove the desiccant, and concentrate to obtain 15.82 g of a white solid, which is a crude product. The crude product was slurried with PE/EA = 8/1 mixture 150 mL; compound M-3 was finally obtained as a white solid (12.0 g, 36.52 mmol, 75% yield).

### Step 3: Synthesis of Compound M-4

The white solid obtained above (12.0 g, 36.89 mmol) was dissolved in methanol (300 mL), replaced with nitrogen, then 10% palladium on carbon (1200 mg) was added, and then replaced with hydrogen, maintaining a hydrogen atmosphere, and stirring the reaction at room temperature. The reaction was stirred for 4.5 hours; LC-MS detection showed complete conversion of starting material. Celite was used for filtration to remove palladium on carbon, and the filter residue was rinsed with a little methanol. The concentrated filtrate was evaporated to dryness, dissolved in dichloromethane, and dried with anhydrous sodium sulfate; concentrated and evaporated to dryness again to obtain an off-white residue. 180 mL of PE/EA+30:1 mixture was added, and the off-white residue obtained from concentration was slurried. Filter, collect the filter cake, suck dry and weigh: Compound M-4 was obtained as an off-white solid powder (7.24 g, 35.29 mmol, 96% yield). ESI-MS m/z: 206.04 [M+H]⁺.

### Step 4: Synthesis of Compound M-5

Compound M-4 (7.24 g, 35.29 mmol) was dissolved in MeCN (80 mL). Under nitrogen protection, turn on stirring, cool to 0° C., then add CuBr₂ (11.82 g, 52.94 mmol); stir for 2 minutes, then dropwise add tert-butyl nitrite (5.46 g, 52.94 mmol), stir for 10 minutes; remove the cooling, the bath was naturally raised to room temperature, and the reaction was stirred for 1 hour. TLC analysis, the conversion of raw materials is complete; post-processing can be carried out. The reaction mixture was added to 400 g of ice-water mixture, then extracted with 400 mL of ethyl acetate, and the layers were separated (saturated aqueous sodium chloride solution facilitated layering a little). The organic phase was washed with saturated aqueous ammonium chloride solution 150 mL×2 and separated. An appropriate amount of anhydrous sodium sulfate was added to dry the organic phase. The desiccant was removed by filtration and concentrated to obtain 12.5 g of a yellow-brown viscous substance. Column chromatography separation and purification (PE/EA = 97/3) finally obtained a white solid M-5 (9.1 g, 33.83 mmol, 96% yield). ESI-MS m/z: 270.11 [M+H]⁺.

### Step 5: Synthesis of Compound M-6

The white solid M-5 (6.0 g, 22.30 mmol) obtained above was added to the reaction flask, then 50 mL of methanol was added, and then 20 mL of an aqueous solution containing LiOH (587 mg) was added at room temperature to stir the reaction, and the reaction was completed in 2 hours. Spin off most of the methanol, then adjust pH = 3, extract with EA (100 mL x 3), after obtaining the organic phase, add an appropriate amount of anhydrous sodium sulfate to dry the organic phase. The drying agent was filtered off and concentrated to obtain a white solid (5.6 g, 98% yield). ESI-MS m/z: 253.11 [M-H]⁻.

### Step 6: Synthesis of Compound M-7

Substrate M-6 (5.5 g, 21.57 mmol) was dissolved in THF (80 mL), then DIEA (8.36 g, 64.71 mmol, 11.27 mL) was added, and carbonyldiimidazole (4.19 g, 25.88 mmol) was added, heated to 50° C. for reaction. Check the reaction until all the ingredients are gone. Then cool to room temperature and set aside. Cool 120 mL of ammonia water to below 0° C., then slowly add the above reaction solution to it, and after the addition, naturally rise to room temperature for reaction. The reaction was monitored until all the starting materials were reacted to completion. EA (120 mL) was added to dilute and washed twice with saturated saline, then spin-dried and separated by column chromatography (PE/EA + 2:1) to obtain a white solid (5.32 g, 20.94 mmol, 97% yield). ESI-MS m/z; 253.96 [M+H]⁺.

### Step 7: Synthesis of Compound M-8

Substrate M-7 (4.61 g, 18.15 mmol) was dissolved in THF (45 mL), then under nitrogen protection, oxalyl chloride (9.21 g, 72.60 mmol, 6.14 mL) was added, heated to reflux for 1 hr, and spin-dried under reduced pressure, washed with DCM, concentrated again, then a solution of 2-isopropyl-4-methyl-3-pyridinamine (4.09 g, 27.22 mmol) in DCM (40 mL) was added, and the reaction was stirred for 1 hr at room temperature. After the reaction finishes, add sodium bicarbonate aqueous solution to the system, then add EA (200 mL) for extraction, after obtaining the organic phase, dry and concentrate, and column chromatography is separated (PE/EA=1:1) to obtain white solid M-8 (6.7 g, 15.57 mmol, 86% yield). ESI-MS m/z: 430.146 [M+H]⁺.

### Step 8: Synthesis of Compound M-9

Substrate M-8 (4.8 g, 11.16 mmol) was dissolved in DME (60 mL), and then NaH (535.5 mg) was slowly added at room temperature. After the system was no longer bubbling, the reaction was heated to 90° C. by microwavefor 10 minutes, after the reaction, saturated sodium bicarbonate solution was added to quench the reaction, then EA (200 mL) was added, the organic phase was washed with saturated brine, and after obtaining the organic phase, it was dried and concentrated, and separated by column chromatography (PE/EA=1:1) to obtain a white solid M-9 (3.96 g, 9.65 mmol, 86% yield). ESI-MS m/z: 410.14 [M+H]⁺.

### Step 9: Synthesis of Compound M-10

Compound M-9 (2.1 g, 5.12 mmol) was dissolved in MeCN (20 mL), then DIPEA (6.62 g, 51.19 mmol, 8.92 mL) was added, followed by POCl₃ (3.92 g, 25.60 mmol, 2.45 mL), The reaction was heated to 60° C. The reaction was detected by LC-MS until all the raw materials disappeared. After the reaction was completed, returning to room temperature, it was concentrated under reduced pressure to obtain a light yellow oily substance, add DCM for rotary evaporation 2 times, and then spin-dried again to obtain an oily substance that was directly used for the next reaction. ESI-MS m/z; 428.14 [M+H]⁺.

### Step 10: Synthesis of Compound M-11

Compound M-10 obtained in the previous step was dissolved in 10 mL of DMF, then DIPEA (1.32 g, 10.22 mmol, 1.78 mL) was added, followed by (3S)-3-methyl piperazine-1-carboxylate tert-butyl ester (1.53 g, 7.66 mmol) dissolved in DMF (10 mL), the reaction was stirred at room temperature, and after the substrate was completely reacted, EA (200 mL) was added, and the organic phase was washed with saturated brine, and the organic phase was obtained and dried. Concentration and column chromatography (PE/EA = 1:1) to obtain M-11 as a white solid (2.9 g, 4.89 mmol. 96% yield). ESI-MS m/z: 592.14 [M+H]⁺.

### Example 1: Synthesis of Compound 1 ((S)-1-(4-Acryloyl-2-Methylpiperazin-1-yl)-11-Fluoro-4-(2-Isopropyl-4-Methyl) Pyridin-3-yl)-4H-Isochromene Bridge [4,3-H] Quinazolin-3(6h)-One)

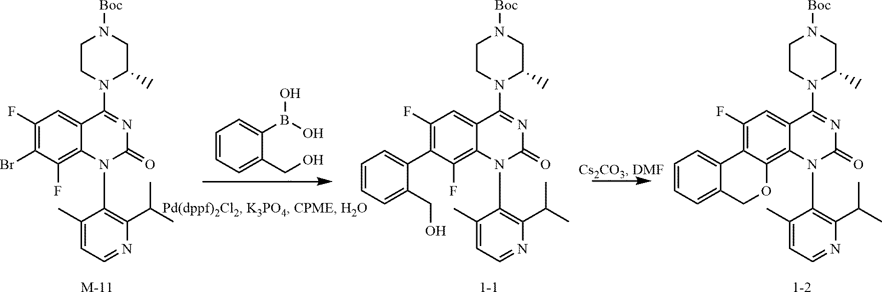

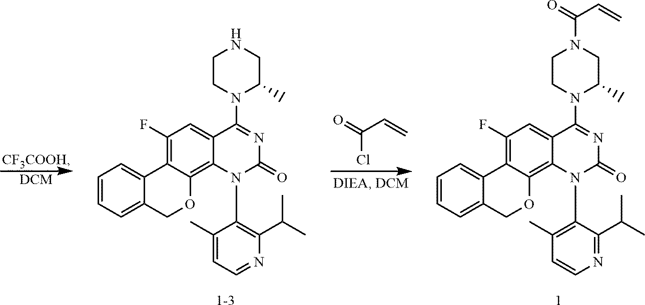

### Step 1: Synthesis of Compound 1-1

Compound M-11 (400 mg, 0.68 mmol) was added to the reaction flask, then boric acid (205 mg, 1.35 mmol) and K₃PO₄ (429.9 mg, 2.03 mmol) were added, CPME and purified water were added to the reaction flask, and then with N₂ swells the oxygen out of the system. After 5 minutes, the catalyst Pd(dppf)Cl₂ (74 mg, 0.1 mmol) was added to the reaction flask, then the stopper was sealed, and the reaction was performed under microwave (90° C., 40 min). After the reaction was completed, cooling to the room, EA was added to dilute the reaction system, and then the organic phase was washed with saturated sodium bicarbonate and saturated brine, and the organic phase was dried and spin-dried, and the product was directly used in the next reaction.

### Step2: Synthesis of Compound 1-2

Compound 1-1 (410 mg, 0.66 mmol) obtained above was dissolved in DMF, then Cs₂CO₃ (431 mg, 1.32 mmol) was added, heated to 125° C. under microwave for 30 minutes, cooled to room temperature after the reaction, and then diluted with EA, washed with saturated sodium bicarbonate and saturated brine, then the organic phase was dried, concentrated, and separated by pre-TLC (EA) preparation to obtain a white foamy solid (169 mg, 42%). ESI-MS m/z: 600.04 [M+H]⁺.

### Step 3: Synthesis of Compounds 1-3

Compound 1-2 (169 mg, 0.28 mmol) obtained above was dissolved in DCM (4 mL), then CF₃COOH (1 mL) was added at room temperature, and the reaction was kept at room temperature for 30 minutes. After the reaction was completed, saturated aqueous sodium bicarbonate solution was added to quench, then EA was added to extract the aqueous phase, the organic phases were combined, dried, concentrated, and directly used in the next reaction.

### Step 4: Synthesis of Compound 1

The compound obtained above was added to dry DCM (4 mL), cooled to 0° C. after heating, and diluted acryloyl chloride (25 mg, 0.26 mmol) was slowly added to the system. After 10 minutes, TLC showed that the substrate reacted complete, saturated sodium bicarbonate was added to quench the reaction, then EA was added to dilute tire reaction system, the aqueous phase was washed with saturated brine, the organic phase was dried, concentrated, and purified by pre-TLC preparation to obtain compound 1 as a white solid (141 mg, 98%). ESI-MS m/z: 554.33.[M+H]⁺: ¹H NMR (500 MHz, CDCl₃) δ 8.49 (d, J= 4.9 Hz, 1 H), 8.02 (d, J= 7.7 Hz, 1 H), 7.41 (t, J = 7.7 Hz, 1 H), 7.36 (t, J = 7.4 Hz, 1 H), 7.18 (d, J = 11.9 Hz, 1 H), 7.05 (d, J= 7.7 Hz, 2 H), 6.60 (dd, J= 32.2, 17.0 Hz, 1 H), 6.39 (d, J = 16.8 Hz, 1 H), 5.80 (d, J = 10.4 Hz, 1 H), 4.73 (d, J = 16.0 Hz, 1 H), 4.57-4.37 (m, 2 H), 2.16-2.06 (m, 3 H), 1.56-1.44 (m, 2 H), 1.41 (d, J = 6.7 Hz, 2 H), 1.21 (t, J = 6.3 Hz, 3 H), 1.12 (d, J = 6.8 Hz, 3 H).

### Example 2: Synthesis of Compound 2 ((S)-8-(4-Acryloyl-2-Methylpiperazin-1-yl)-12-Fluoro-11-(2-Isopropyl-4-Methyl) Pyridin-3-yl)-5H-Isochromene[3,4-g] Quinazolin-10(11H)-One)

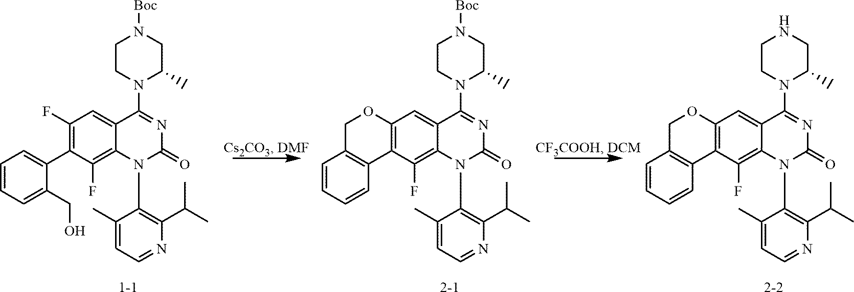

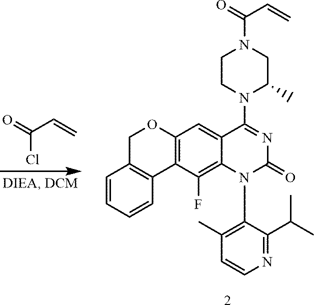

### Step 1: Synthesis of Compound 2-1

Compound 1-1 (410 mg, 0.66 mmol) obtained above was dissolved in DMF, then Cs₂CO₃ (431 mg, 1.32 mmol) was added, heated to 125° C. under microwave for 30 minutes, cooled to room temperature after the reaction, and then diluted with EA, washed with saturated sodium bicarbonate and saturated brine, then the organic phase was dried, concentrated, and separated by pre-TLC (EA) preparation to obtain a white foamy solid (170 mg, 42%). ESI-MS m/z: 600.04 [M+H]⁺.

### Step 2: Synthesis of Compound 2-2

Compound 2-1 (169 mg, 0.28 mmol) obtained above was dissolved in DCM (4 mL), then CF₃COOH (1 mL) was added at room temperature, and the reaction was kept at room temperature for 30 minutes. After the reaction was completed, saturated aqueous sodium bicarbonate solution was added to quench, then EA was added to extract the aqueous phase, the organic phases were combined, dried, concentrated, and directly used in the next reaction.

### Step 3: Synthesis of Compound 2

The compound obtained above was added to dry DCM (4 mL), cooled to 0° C. after heating, and diluted acryloyl chloride (25 mg, 0.26 mmol) was slowly added to the system. After 10 minutes, TLC showed that the substrate reacted complete, saturated sodium bicarbonate was added to quench the reaction, then EA was added to dilute the reaction system, the aqueous phase was washed with saturated brine, the organic phase was dried, concentrated, and purified by pre-TLC preparation to obtain white solid compound 2 (115 mg, 84%).

ESI-MS m/z: 554.33 [M+H]⁺; ¹H NMR (500 MHz, CDCl₃) δ 8.55 (d, J = 4.9 Hz, 1 H), 7.73 (d, J = 7.7 Hz, 1 H), 7.38 (t, J = 7.3 Hz, 1 H), 7.34 (t, J = 7.6 Hz, 1 H), 7.25-7.17 (m, 2 H), 7.11 (d, J = 4.9 Hz, 1 H), 6.60 (dd, J = 35.6, 13.4 Hz, 1 H), 6.39 (d, J = 16.7 Hz, 1 H), 5.79 (d, J = 10.5 Hz, 1 H), 5.17-5.05 (m, 2 H), 2.15 (d, J = 6.5 Hz, 3 H), 1.25 (dd, J = 6.9, 4.0 Hz, 4 H), 1.17 (d, J = 7.0 Hz, 4 H).

### Example 3: Synthesis of Compound 3 ((S)-1-(Acryloyl-2-Methylpiperazin-1-yl)-10,11-Difluoro-4-(2-Isopropyl-4-Methyl) Pyridin-3-yl)-4H-Isochromene[4,3-h] Quinazolin-3(6H)-One)

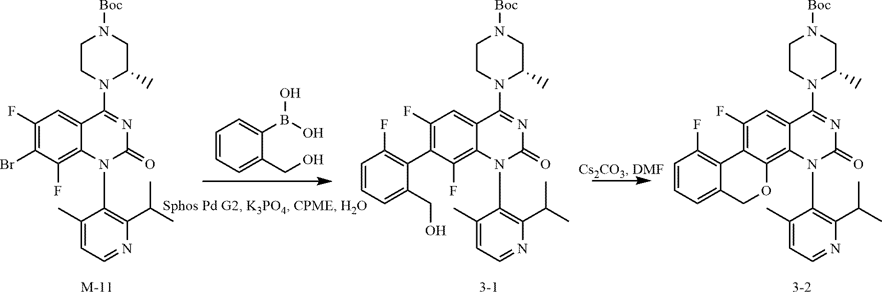

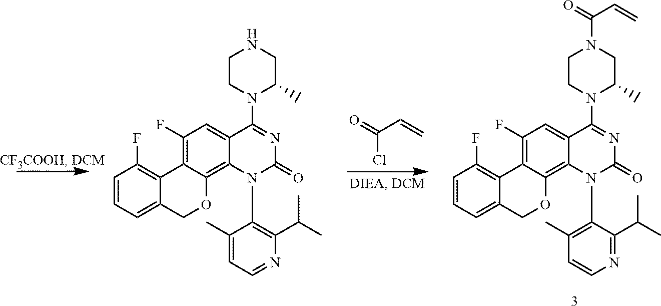

### Step 1: Synthesis of Compound 3-1

Compound M-11 (603 mg, 1.02 mmol) was added to the reaction flask, then boric acid (345.9 mg, 2.04 mmol) and K₃PO₄ (1.3 g, 6.11 mmol) were added, CPME and purified water were added to the reaction flask, and then with N₂ swells the oxygen out of the system. After 5 minutes, the catalyst SPhos Pd G₂ (147 mg, 0.2 mmol) was added to the reaction flask, then the stopper was sealed, and the reaction was performed under microwave (90° C., 40 min). After the reaction was completed, after cooling to the room, EA was added to dilute the reaction. The system was then washed with saturated sodium bicarbonate and the organic phase was washed with saturated brine, then the organic phase was dried and spin-dried, and used directly in the next reaction.ESI-MS m/z: 638.44 [M+H]⁺.

### Step 2: Synthesis of Compound 3-2

Compound 3-1 (535 mg, 0.84 mmol) obtained above was dissolved in DMF, then Cs₂CO₃ (820 mg, 2.52 mmol) was added, heated to 125° C. under microwave for 30 minutes, cooled to room temperature after the reaction, and then EA was added diluted, washed with saturated sodium bicarbonate and saturated brine, then the organic phase was dried, concentrated, and separated by pre-TLC (EA) preparation to obtain a white foamy solid (360 mg, 67%).ESI-MS m/z: 618.37 [M+H]⁺.

### Step 3: Synthesis of Compound 3-3

Compound 3-2 (349 mg, 0.56 mmol) obtained above was dissolved in DCM (6 mL), then CF₃COOH (2 mL) was added at room temperature, and the reaction was kept at room temperature for 30 minutes. After the reaction was completed, saturated aqueous sodium bicarbonate solution was added to quench, then EA was added to extract the aqueous phase, the organic phases were combined, dried, concentrated, and directly used in the next reaction.

### Step 4: Synthesis of Compound 3

The compound obtained above was added to dry DCM (4 mL), cooled to 0° C. after heating, and diluted acryloyl chloride (51 mg, 0.56 mmol) was slowly added to the system. After 10 minutes, TLC showed that the substrate reacted complete, saturated sodium bicarbonate was added to quench the reaction, then EA was added to dilute the reaction system, the aqueous phase was washed with saturated brine, the organic phase was dried, concentrated, and purified by pre-TLC preparation to obtain compound 3 as a white solid (305 mg, 94%) . ESI-MS m/z: 572.3 [M+H]⁺; ¹H NMR (500 MHz, CDCl₃) δ 8.49 (d, J= 4.9 Hz, 1 H), 7.38 (td, J= 7.9, 4.8 Hz, 1 H), 7.24-7.13 (m, 2 H), 7.07 (t, J= 4.2 Hz, 1 H), 6.92 (d, J= 7.4 Hz, 1 H), 6.62 (dt, J = 38.5, 14.2 Hz, 1 H), 6.40 (d, J= 16.8 Hz, 1 H), 5.80 (d, J= 10.5 Hz, 1 H), 4.52-4.38 (m, 3 H), 2.11 (d, J= 11.8 Hz, 3 H), 1.41 (d, J= 6.2 Hz, 2 H), 1.22 (t, J= 7.1 Hz, 3 H), 1.11 (t, J= 7.2 Hz, 3 H).

### Example 4: Synthesis of Compound 4 ((S)-8-(Acryloyl-2-Methylpiperazin-1-yl)-1,12-Difluoro-11-(2-Isopropyl-4-Methylpyridin-3-yl)-5H-Isochromene[3,4-g] Quinazolin-10(11H)-One)

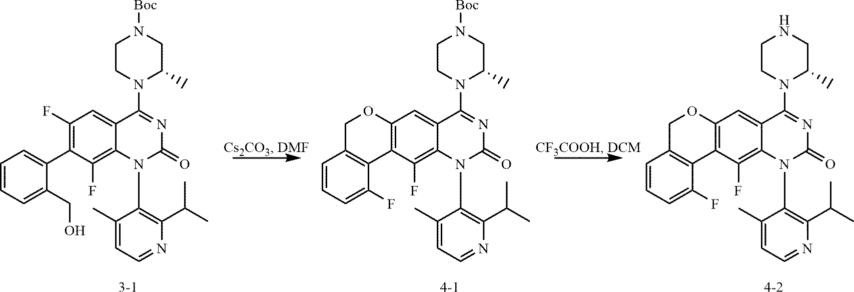

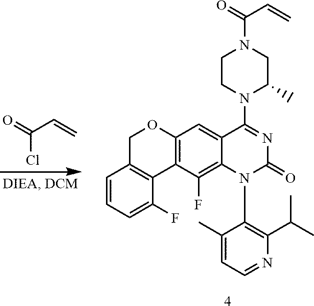

### Step 1: Synthesis of Compound 4-1

Compound 3-1 (535 mg, 0.84 mmol) obtained above was dissolved in DMF, then Cs₂CO₃ (820 mg, 2.52 mmol) was added, heated to 125° C. under microwave for 30 minutes, cooled to room temperature after the reaction, and then EA was added to dilute, washed with saturated sodium bicarbonate and saturated brine, then the organic phase was dried, concentrated, and separated by pre-TLC (EA) to obtain a white foamy solid (170 mg, 33%).ESI-MS m/z: 618.37 [M+H]⁺.

### Step 2: Synthesis of Compound 4-2

Compound 4-1 (170 mg, 0.28 mmol) obtained above was dissolved in DCM (6 mL), then CF₃COOH (2 mL) was added at room temperature, and the reaction was kept at room temperature for 30 minutes. After the reaction was completed, saturated aqueous sodium bicarbonate solution was added to quench ,, then EA was added to extract the aqueous phase, the organic phases were combined, dried, concentrated, and directly used in the next reaction.

### Step 3: Synthesis of Compound 4

The compound obtained above was added to dry DCM (4 mL), cooled to 0° C. after heating, and diluted acryloyl chloride (25 mg, 0.28 mmol) was slowly added to the system. After 10 minutes, TLC showed that the substrate reacted Complete, saturated sodium bicarbonate was added to quench the reaction, then EA was added to dilute the reaction system, the aqueous phase was washed with saturated brine, the organic phase was dried, concentrated, and purified by pre-TLC preparation to obtain compound 4 as a white solid (110 mg, 68%) .ESI-MS m/z: 572.3 [M+H]⁺; ¹H NMR (500 MHz, CDCl₃) δ 8.54 (d, J= 4.9 Hz, 1 H), 7.40 (td, J= 7.9, 4.8 Hz, 1 H), 7.24 (s, 1 H), 7.09 (t, J= 6.3 Hz, 3 H), 6.61 (ddd, J= 39.8, 14.7, 6.6 Hz, 1 H), 6.39 (d, J = 16.8 Hz, 1 H), 5.79 (d, J = 10.5 Hz, 1 H), 2.13 (d, J= 6.9 Hz, 3 H), 1.57-1.44 (m, 2 H), 1.39 (d, J = 6.7 Hz, 1 H), 1.25 (t, J= 5.9 Hz, 3 H), 1.18 (q, J= 6.8, 6.4 Hz, 4 H).

### Example 5: Synthesis of Compound 5 ((S)-1-(Acryloyl-2-Methylpiperazin-1-yl)-10-Chloro-11-Fluoro-4-(2-Isopropyl-4-Methylpyridin-3-yl)-4H-Isochromene[4,3-h] Quinazolin-3(6H)-One)

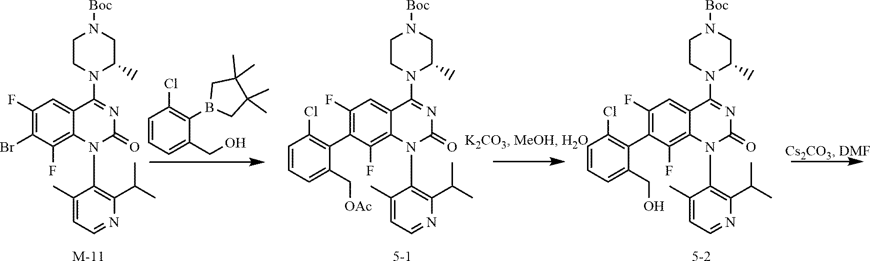

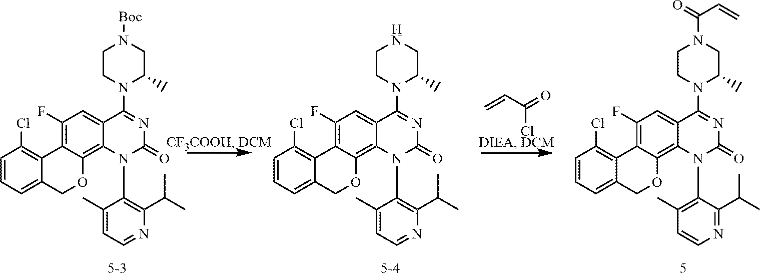

For the specific synthesis steps of compound 5, refer to Example 3.ESI-MS m/z: 588.3 [M+H]⁺; ¹H NMR (500 MHz, CDCl₃)δ 8.49 (d, J = 4.9 Hz, 1 H), 7.49 (d, J = 8.0 Hz, 1 H), 7.31 (t, J = 7.8 Hz, 1 H), 7.21 (d, J = 10.0 Hz, 1 H), 7.05 (dd, J = 9.3, 5.9 Hz, 2 H), 6.62 (dt, J = 37.2, 13.9 Hz, 1 H), 6.40 (d, J = 16.8 Hz, 1 H), 5.80 (d, J = 10.4 Hz, 1 H), 4.50-4.35 (m, 2 H), 2.11 (d, J = 10.5 Hz, 3 H).

### Example 6: Synthesis of Compound 6 ((S)-8-(Acryloyl-2-Methylpiperazin-1-yl)-1-Chloro-12-Fluoro-11-(2-Isopropyl-4-Methylpyridin-3-yl)-5 H-Isochromene[3,4-g] Quinazolin-10(11 H)-one)

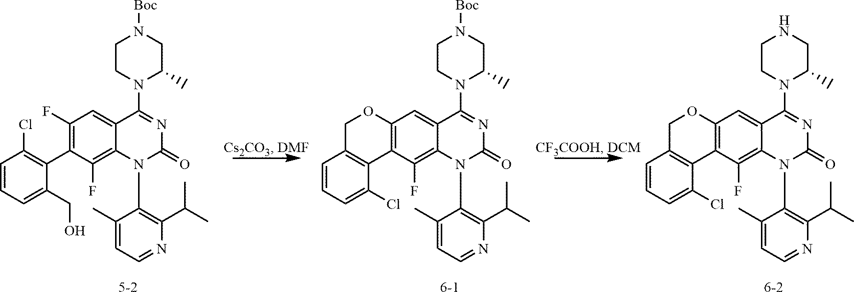

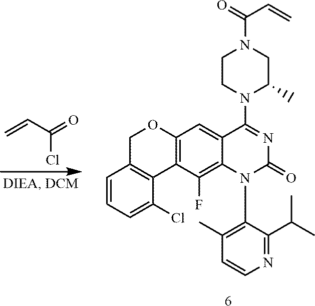

The specific synthesis steps of compound 6 refer to Example 4.ESI-MS m/z: 588.3 [M+H]⁺; ¹H NMR (500 MHz, CDCl₃) δ 8.53 (d, J = 4.9 Hz, 1 H), 7.43 (d, J = 8.1 Hz, 1 H), 7.33 (t, J = 7.8 Hz, 1 H), 7.20 (d, J = 7.4 Hz, 1 H), 7.09 (d, J= 4.9 Hz, 1 H), 6.59 (dd, J = 31.6, 16.4 Hz, 1 H), 6.39 (d, J =16.8 Hz, 1 H), 5.79 (d, J = 10.5 Hz, 1 H).

### Example 7: Synthesis of Compound 7 (10-((S)-4-(Acryloyl-2-Methylpiperazin-1-yl)-8-Fluoro-13-(2-Isopropyl-4-Methyl) Pyridin-3-yl)-2,3-Dihydrobenzo [4,5] Oxodipine [3,2-h] Quinolin-12(13H)One)

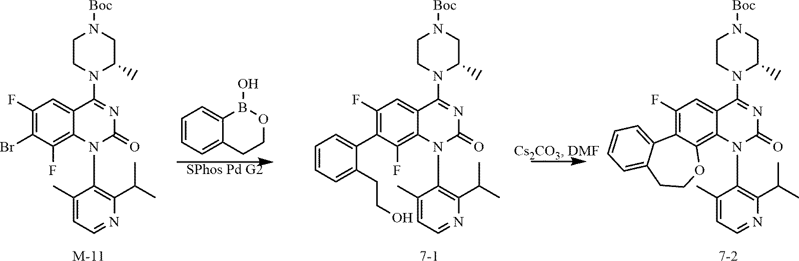

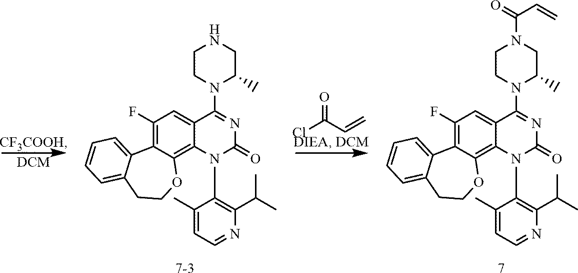

For the specific synthesis steps of compound 7, refer to Example 3.ESI-MS m/z: 568.3 [M+H]⁺; ¹H NMR (500 MHz, CDCl₃)δ 8.47 (d, J = 4.9 Hz, 1 H), 7.54 (t, J = 5.7 H_{z.} 1 H), 7.44-7.30 (m, 3 H), 7.22 (d, J =7.2 Hz, 1 H), 7.08 (d, J =4.9 Hz, 1 H), 6.61 (dd, J =38.6, 12.8 Hz, 1 H), 6.40 (d, J = 16.7 Hz, 1 H), 5.80 (d, J = 10.5 Hz, 1 H), 2.18 (d, J = 3.5 Hz, 3 H), 1.23-1.04 (m, 6 H).

### Example 8: Synthesis of Compound 8

((S)-4-(acryloyl-2-methylpiperazin-1-yl)-13-fluoro-12-(2-isopropyl-4-methyl) pyridin-3-yl)-5,6-dihydrobenzo [4,5]oxodipine [2,3-h]quinolin-11(12H)one)

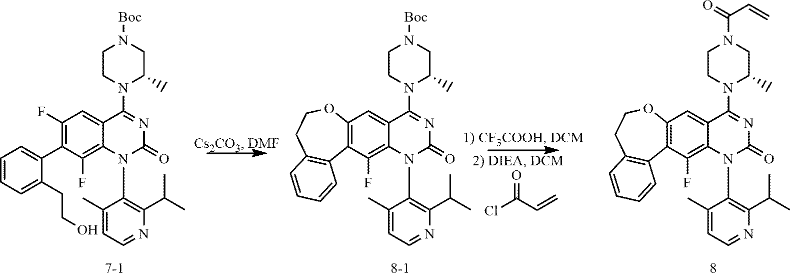

For the specific synthesis steps of compound 8, refer to Example 4.ESI-MS m/z: 568.3 [M+H]⁺; ¹H NMR (500 MHz, CDCl₃) δ8.50 (d, J = 4.9 Hz, 1 H), 7.34 (dq, J = 22.7, 7.8 Hz, 5 H), 7.07 (d, J = 4.9 Hz, 1 H), 6.73 -6.51 (m, 1 H), 6.40 (d, J = 16.8 Hz, 1 H), 5.80 (d, J = 10.5 Hz, 1 H).

### Example 9: Synthesis of Compound 9

((S)(Acryloyl-2-methylpiperazinyl)-11-fluoro-10-hydroxy-4-(2-isopropyl-4-methylpyridin-3-yl) -4 H-isochromene[4,3-h] quinazolin-3(6 H)-one)

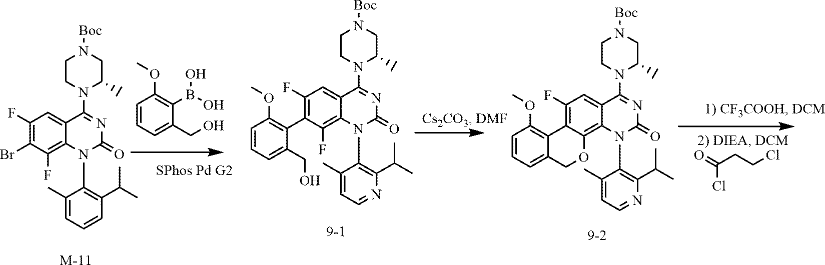

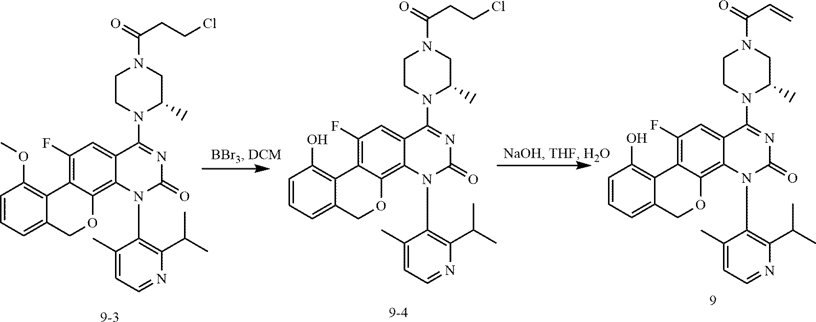

### Step 1: Synthesis of Compounds 9-4

Compound 9-3 was synthesized with reference to Example 3. Compound 9-3 (100 mg, 0.161 mmol) was dissolved in DCM (5 mL), then cooled to 0° C., BBr₃ (404 mg, 1.61 mmol) was slowly added, and the reaction was maintained at this temperature for 1 hour. Check the reaction until the raw material is completely consumed, then at this temperature, add saturated aqueous sodium bicarbonate solution to quench the reaction, then add DCM to dilute the reaction, wash with water, dry the organic phase, spin dry, and proceed directly to the next reaction. (96 mg, 98% yield).ESI-MS m/z: 606.33 [M+H]⁺.

### Step 2: Synthesis of Compound 9

The compound obtained above was dissolved in THF (3 mL), then 0.2 mL of 6 N NaOH solution was added, and the reaction was stirred at room temperature for 30 minutes. After the reaction of the substrate was completed, saturated sodium bicarbonate was used to neutralize the strong base in the system, and DCM was added for extraction twice,then combine the organic phases, dry, spin dry, and prepare and separate by HPLC to obtain the target product 9 (54 mg, 59% yield).ESI-MS m/z: 570.3 [M+H]⁺; ¹H NMR (500 MHz, CDCl₃) δ 8.52 (d, J = 5.0 Hz, 1 H), 7.22 (q, J = 11.5, 9.7 Hz, 2 H), 7.12 (d, J = 5.0 Hz, 1 H), 7.02 (d, J = 8.2 Hz, 1 H), 6.64 (d, J = 7.4 Hz, 1 H), 6.43 (d, J = 16.7 Hz, 1 H), 5.83 (d, J = 10.5 Hz, 1 H), 5.30 (s, 2 H), 2.13 (d, J = 9.6 Hz, 3 H), 1.24 (t, J= 7.6 Hz, 3 H), 1.12 (d, J = 6.9 Hz, 3 H).

### Example 10: Synthesis of Compound 10 ((S)-8-(Acryloyl-2-Methylpiperazin-1-yl)-12-Fluoro-1-Hydroxy-1l-(2-Isopropyl-4-Methylpyridin-3-yl)-5H-Isochromene[3,4-g]Quinazolin-10(11H)-One)

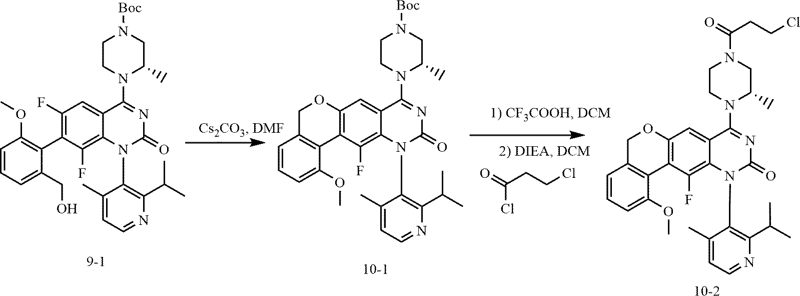

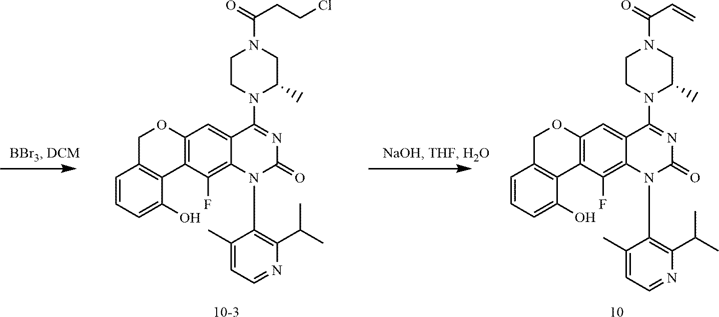

The specific synthesis steps of compound 10 refer to Example 9.ESI-MS m/z: 570.3 [M+H]⁺:

### Example 11: Synthesis of Compound 11 ((S)-1-(Acryloyl-2-Methylpiperazin-1-yl)-l1-Fluoro-10-Methoxy-4-(2-Isopropyl-4-Methylpyridin-3-yl)-4H-Isochromene[4,3-h] Quinazolin-3(6H)-one)

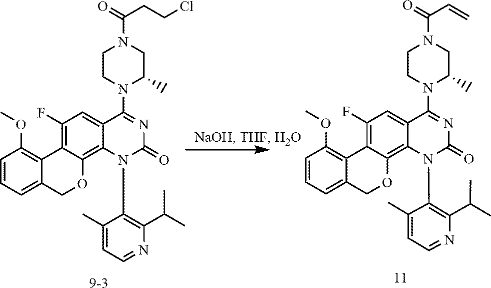

The specific synthesis steps of compound 11 refer to Example 9.ESI-MS m/z: 584.4 [M+H]⁺.

### Example 12: Synthesis of Compound 12 ((S)-8-(Acryloyl-2-Methylpiperazin-1-yl)-12-Fluoro-1-Methoxy-11-(2-Isopropyl-4-Methylpyridin-3-yl)-5H-Isochromene[3,4-g] Quinazolin-10(11H)-One)

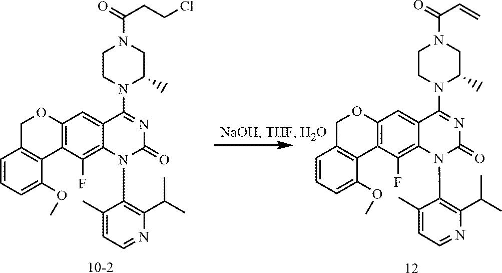

The specific synthesis steps of compound 12 refer to Example 9.ESI-MS m/z: 584.4 [M+H]⁺ .

### Example 13: Synthesis of Compound (S)-1-(4-Acryloyl-2-Methylpiperazin-1-yl)-11-Chloro-10-Fluoro-4-(2-Isopropyl-4-Methylpyridin-3-yl)-4H-Isochromene[4,3-h] Quinazolin-3(6H)-One

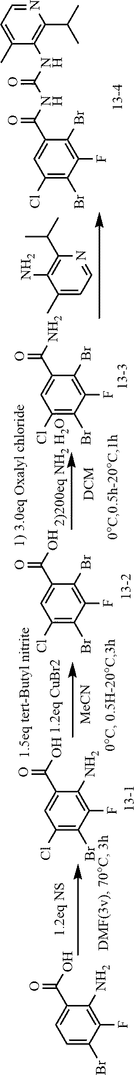

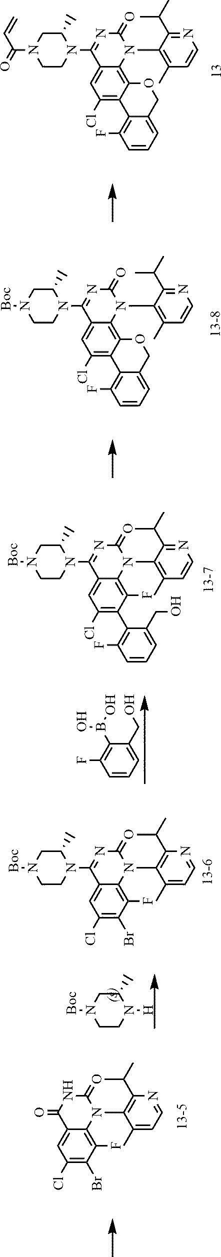

### Step 1: Synthesis of Compound 13-1

At room temperature, the compound 2-amino-4-bromo-3-fluorobenzoic acid (10 g, 42.73 mmol) was dissolved in DMF (30 mL), NCS (6.85 g, 51.28 mmol) was added, and the reaction was carried out at 70° C. 3 hs. The reaction solution was cooled to room temperature, added to 150 ml of ice water, and a light yellow solid was precipitated, which was filtered, the filter cake was washed with ice water (2 *20 ml), and the filter cake was vacuum-dried (-0.1 Mpa, 50° C., 4 h), obtained yellow solid compound (11.2 g, 41.72 mmol, 97.63% yield). ESI-MS m/z: 267.9[M+H]⁺.

### Step 2: Synthesis of Compound 13-2

At room temperature, compound 13-1 (11.2 g, 41.72 mmol) was dissolved in MeCN (146 mL), under nitrogen protection, stirring was turned on, then copper bromide (11.18 g, 50.06 mmol) was added, the ice-water bath was cooled, to 0° C., then dropwise added the MeCN (52 mL) solution of tert-butyl nitrite (6.45 g, 62.58 mmol); After dropping, be incubated at 0-5° C. and stirred for 30 minutes, then remove the cold bath, and naturally rise to room temperature, and the reaction was stirred for 3 hs. The reaction solution was added to 600 mL of cold saturated aqueous ammonium chloride solution, 500 mL of ethyl acetate was added, the layers were separated, the organic phase was separated, and the aqueous phase was extracted with 200 mL of ethyl acetate. Wash with 300 mL of saturated sodium chloride solution for 2 times, dry the organic phase with anhydrous sodium sulfate, filter, and concentrate the filtrate under reduced pressure. A light brown solid (12.19 g, 36.68 mmol, 87.92%) yield) was obtained which was used directly in the next step.ESI-MS m/z: 330.8 [M+H]⁺.

### Step 3: Synthesis of Compound 13-3

At room temperature, compound 13-2 (12.19 g, 36.68 mmol) was dissolved in DCM (122 mL), and 4 drops of DMF were added dropwise; then cooled in an ice-water bath, oxalyl chloride (13.97 g, 110.03 mmol, 9.31 mL) was added dropwise ) in DCM (14 mL); then the reaction was stirred for half an hour, the reaction solution was concentrated under reduced pressure, and add 20 mL of DCM for rotary evaporation, repeat 2 times; ammonium hydroxide (15 M, 684.66 mL) was cooled in an ice-water bath; add THF (60 mL) dropwise to dissolve the intermediate residue, and the reaction was naturally raised to room temperature for 1 hs. Add 300 mL of ethyl acetate, 300 mL of saturated aqueous sodium chloride solution, separate the layers, and separate the organic phase; the aqueous phase is then extracted with 150 mL of ethyl acetate, and the layers are separated; the organic phases are combined, and then washed with 100 ml saturated aqueous sodium chloride solution for 2 times; add anhydrous sodium sulfate to dry; filter, and concentrate the filtrate under reduced pressureto obtain brown solid (10.05 g, 30.33 mmol, 82.69% yield) EST-MS m/z: 329.8 [M+H]⁺.

### Step 4: Synthesis of Compound 13-4

At room temperature, compound 13-3 (9.6 g, 28.97 mmol) was dissolved in THF (192 mL), under nitrogen protection, oxalyl chloride (14.71 g, 115.88 mmol, 9.81 mL) was added and heated to 70° C. for reflux reaction for 2 hs; Concentrate under reduced pressure to remove THF and oxalyl chloride, and evaporate to dryness with anhydrous DCM (15 mL×2). The concentrated residue was dissolved in DCM (20 ml), a solution of 2-isopropyl-4-methylpyridin-3-amine (4.35 g, 28.97 mmol) in DCM (160 mL) was added dropwise, and the reaction was stirred at room temperature for 2 hours. The reaction mixture was diluted with DCM (100 mL), washed with saturated aqueous ammonium chloride solution (100 mL) and saturated aqueous sodium bicarbonate solution (100 mL) and separated; then washed with saturated aqueous sodium chloride solution (100 mL) and separated ; dried with anhydrous sodium sulfate, filtered, and the filtrate was concentrated under reduced pressure to obtain a brown solid compound (13.3 g, 26.20 mmol, 90.44% yield), which was directly for the next step.ESI-MS m/z: 505.9 [M+H]⁺.

### Step 5: Synthesis of Compound 13-5

At room temperature, compound 13-4 (2000 mg, 3.94 mmol) was dissolved in dimethoxyethane (20 mL), under nitrogen protection, NaH (472.83 mg, 11.82 mmol, 60% purity) was added, and stirred at room temperature for 2 minutes, until the bubbles are reduced to a trace amount, microwave at 90° C. for 10 minutes. Add 300 ml of ice water, adjust the pH to 5-6 with concentrated hydrochloric acid, extract with EA (3*200 ml), no product in the aqueous phase, combine the organic phases, dry with anhydrous sodium sulfate, filter, and concentrate the filtrate under reduced pressure. A tan solid (1.6 g, 3.75 mmol, 95.17% yield) was obtained, which was taken directly to the next step.ESI-MS m/z: 426.0 [M+H]⁺.

### Step 6: Synthesis of Compound 13-6

At room temperature, compound 13-5 (10.3 g, 24.14 mmol) was added to the reaction flask, ACN (103 mL) was added to dissolve, then POCI₃ (7.40 g, 48.28 mmol) and DIPEA (12.48 g, 96.56 mmol, 16.82 mL) were added.and then react at 80° C. for 1 h. The solvent was spun dry, and anhydrous DCM (15 ml *2) was added twice to remove the remaining POC1₃. The product was obtained as a brown oil (10.5 g, 23.59 mmol, 97.72% yield). The above brown oil (10.5 g, 23.59 mmol) was added to the reaction flask, DMF (105 mL) was added to dissolve, DIPEA (9.15 g, 70.77 mmol, 12.33 mL) and (3S)-3-methylpiperazine-1-carboxylate tert-butyl ester (6.14 g, 30.67 mmol) was added to the reaction flaskreacted at 20° C. for 0.5 h. Add 500 ml ice water, add 300 ml EA, separate layers, the aqueous phase was extracted with 150 ml of EA, combine the organic phases, wash with saturated brine (2*300 ml), dry, filter, and concentrate under reduced pressure. A brown-black solid product (13.0 g, 21.35 mmol, 90.50% yield) was obtained, which was directly used in the next step.ESI-MS m/z: 608.1 [M+H]⁺.

### Step 7: Synthesis of Compound 13-7

At room temperature, compound 13-6 (1000 mg, 1.64 mmol), [2-fluoro-6-(hydroxymethyl) phenyl] boronic acid (507.44 mg, 2.99 mmol), CPME (12 mL) were added to the reaction flask. , stirring to dissolve, adding K₃PO₄ (950.72 mg, 4.48 mmol), H₂O (1.5 mL), SPhos Pd G2 (161.15 mg, 223.94 umol), nitrogen protection, 110° C. microwave reaction for 50 minutes. Dilute with 20 ml of EA, filter through celite, add 20 ml of water to the filtrate, separate the layers, wash the organic phase with saturated brine (1*15 ml), dry with anhydrous sodium sulfate, filter, and concentrate the filtrate under reduced pressure. The residue was purified by silica gel column (EA:PE=10% to 100%).to obtainbrown solid product (550 mg, 840.79 umol, 56.32% yield).ESI-MS m/z: 654.2 [M+H]⁺.

### Step 8: Synthesis of Compound 13-8

Compound 13-7 (550 mg, 840.79 umol) was added to the reaction flask, DMF (11 mL) was added, stirred to dissolve, Cs₂CO₃ (1.10 g, 3.36 mmol) was added, under nitrogen protection, and the reaction was carried out in a microwave at 100° C. for 40 minutes. Filter through celite, add 50 ml of water to the filtrate, add 30 ml of EA, separate layers, wash the organic phase with saturated brine (20 ml) for 2 times, dry withanhydrous sodium sulfate, filter, and concentrate under reduced pressure. The residue was purified by silica gel column (EA:PE=10% to 100%) to obtain a brownish yellow solid product (300 mg, 473.08 umol, 56.27% yield). ESI-MS m/z: 634.3 [M+H]⁺.

### Step 9: Synthesis of Compound 13

Compound 13-8 (50 mg, 78.85 uL) was added to the reaction flask, DCM (0.7 mL) was added, stirred to dissolve, TFA (539.41 mg, 4.73 mmol, 350.27 uL) was added, and the reaction was carried out at 20°Cfor 0.5 hour. The reaction solution was concentrated under reduced pressure, anhydrous DCM (3 ml) was added for rotary evaporation, which was repeated 2 times, added anhydrous DCM (1 mL), added DIPEA (509.51 mg, 3.94 mmol, 686.67 uL), cooled to 0° C., a solution of acryloyl chloride (8.56 mg, 94.62 umol) in 0.25 ml anhydrous DCM was added dropwise and reacted at 0° C. for 10 minutes.The reaction solution was diluted with 10 ml of DCM, washed with saturated brine (3*15 ml), the organic phase was dried with anhydrous sodium sulfate, filtered, and concentrated under reduced pressure. The residue was purified by prep-HPLC to obtain off-white solid powder compound 13 (18.9 mg, 32.14 umol, 40.76% yield),The characteristic peaks of this compound are as follows: ¹H NMR (500 MHz, CDCl₃) δ 8.51 (d, J= 4.9 Hz, 1 H), 7.52 (s, 1 H), 7.40 (td, J = 7.9, 4.9 Hz, 1 H), 7.22-7.14 (m, 1 H), 7.07 (d, J = 16.7 Hz, 1 H), 6.95 (d, J= 7.4 Hz, 1 H), 6.69-6.52 (m, 1 H), 6.40 (d, J= 16.7 Hz, 1 H), 5.80 (d, J = 10.6 Hz, 1 H). ESI-MS m/z: 588.2 [M+H]⁺.

### Example 14: Synthesis of Compound (S)-1-(4-Acryloyl-2-Methylpiperazin-1-yl)-10-Fluoro-4-(2-Isopropyl-4-Methylpyridine-3-yl)-11-Vinyl-4H-Isochromene[4,3-h] Quinazolin-3(6H)-One

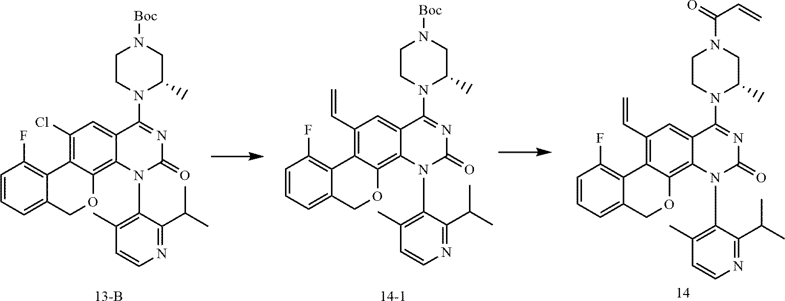

### Step 1: Synthesis of Compound 14-1

In room temperature, potassium vinyltrifluoroborate (158.42 mg, 1.18 mmol), K₃PO₄ (301.25 mg, 1.42 mmol), PdCl₂(dppf)₂DCM (19.32 mg, 23.65 umol) were added to a solution of compound 13-8 (75 mg, 1118.27 umol) in dioxane (3 mL) and water (0.4 mL), nitrogen protection, microwave reaction at 140° C. for 1.5 hs. 20 ml of EA, 10 ml of water were added, the layers were separated, the organic phase was washed with saturated brine (2*15 ml), dried over anhydrous sodium sulfate, filtered, and concentrated under reduced pressure. The residue was purified by Prep-TLC (EA) to obtain a brown solid (48 mg, 76.71 umol, 64.86% yield).ESI-MS m/z: 626.3 [M+H]⁺.

### Step 2: Synthesis of Compound 14

Compound 14-1 (63 mg, 100.68 uL) was added to the reaction flask, DCM (0.9 mL) was added, stirred to dissolve, TFA (688.79 mg, 6.04 mmol, 448.72 uL) was added, and the reaction was carried out at 20° C. for 0.5 hour. The reaction solution was concentrated under reduced pressure, anhydrous DCM (3 ml) was added for rotary evaporation, which was repeated 2 times., added anhydrous DCM (1.0 mL), DIPEA (650.61 mg, 5.03 mmol, 876.83 uL), cooled to 0° C., dropwise added acryloyl chloride (10.94 mg, 120.82 umol), reacted at 0° C. for 10 minutes. The reaction solution was diluted with 10 ml of DCM, washed with saturated brine (3*15 ml), the organic phase was dried over anhydrous sodium sulfate, filtered, and concentrated under reduced pressure. The residue was purified by pre-HPLCto obtain a pale yellow solid powder (26.1 mg, 45.03 umol, 44.72% yield). The characteristic peaks of this compound are as follows:¹H NMR (500 MHz, CDCl₃) δ 8.48 (d, J= 4.9 Hz, 1 H), 7.71 (d, J = 15.0 Hz, 1 H), 7.37 (ddd, J = 12.7, 8.9, 4.9 Hz, 1 H), 7.18-7.03 (m, 2 H), 6.94 (d, J = 7.4 Hz, 1 H), 6.68 (dt, J = 54.2, 23.0 Hz, 2 H), 6.40 (d, J = 16.8 Hz, 1 H), 5.76 (dd, J = 33.6, 14.2 Hz, 2 H), 5.32 (d, J = 10.7 Hz, 1 H). ESI-MS m/z: 580.3 [M+H]^{÷}

### Example 15: Synthesis of Compound (S)-1-(4-Acryloyl-2-Methylpiperazin-1-yl)-10-Fluoro-4-(2-Isopropyl-4-Methylpyridine-3-yl)-11-Methyl-4H-Isochromene[4,3-h] Quinazolin-3(6H)-One

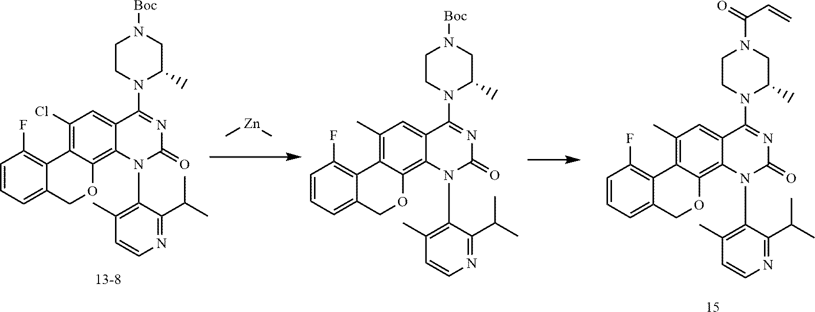

### Step 1: Synthesis of Compound 15-1

At room temperature, trimethylboroxane (296.93 mg, 1.18 mmol, 50% purity), K₃PO₄(251.04 mg, 1.18 mmol), SPhos Pd G2 (17.02 mg, 23.65 umol) were added to a solution of compound 13-8(75 mg, 118.27 umol) in dioxane (3.6 mL) and water(0.45 mL),nitrogen protection, microwave reaction at 140° C. for 0.5 hs. Add 10 ml EA, 5 ml water, and separate the layers. The aqueous phase has no product discarded. The organic phase is washed with saturated brine (2*10 ml), dried over anhydrous sodium sulfate, filtered, and concentrated under reduced pressure. The residue was purified by pre-TLC (EA:PE=85%), obtained a brown solid product (41 mg, 66.81 umol, 49.60% yield). ESI-MS m/z: 614.3 [M+H]⁺.

### Step 2: Synthesis of Compound 15

Compound 15-1 (53.00 mg, 86.36 uL) was added to the reaction flask, DCM (770.00 uL) was added, stirred to dissolve, TFA (590.80 mg, 5.18 mmol, 383.63 uL) was added, and the reaction was carried out at 20° C. for 0.5 hour. The reaction solution was concentrated under reduced pressure, anhydrous DCM (3 ml) was added for rotary evaporation, which was repeated 2 times, anhydrous DCM (1.0 mL) and DIPEA (558.05 mg, 4.32 mmol, 752.09 uL) were added, cooled to 0° C., acryloyl chloride (9.38 mg, 103.63 umol) was added dropwise and reacted at 0° C. for 10 minutes. The reaction solution was diluted with 10 ml of DCM, washed with saturated brine (3*10 ml), the organic phase was dried over anhydrous sodium sulfate, filtered, and concentrated under reduced pressure. The residue was purified by pre-HPLC. The product was obtained as a white solid powder (11.5 mg, 20.26 umol, 23.46% yield). The characteristic peaks of this compound are as follows:¹H NMR (500 MHz, CDCl₃) δ 8.47 (d, J = 4.8 Hz, 1 H), 7.39-7.29 (m, 2 H), 7.18-7.10 (m, 1 H), 7.05 (t, J = 4.5 Hz, 1 H), 6.94 (d, J = 7.4 Hz, 1 H), 6.62 (d, J = 38.1 Hz, 1 H), 6.39 (d, J = 16.7 Hz, 1 H), 5.79 (d, J = 10.8 Hz, 1 H). ESI-MS m/z: 568.3 [M+H]⁺.

### Example 16: Synthesis of Compound (S)-1-(4-Acryloyl-2-Methylpiperazin-1-yl)-10-Fluoro-4-(2-Isopropyl-4-Methylpyridine-3-yl)-11-(Trifluoromethyl)-4H-Isochromene[4,3-h] Quinazolin-3(6H)-One

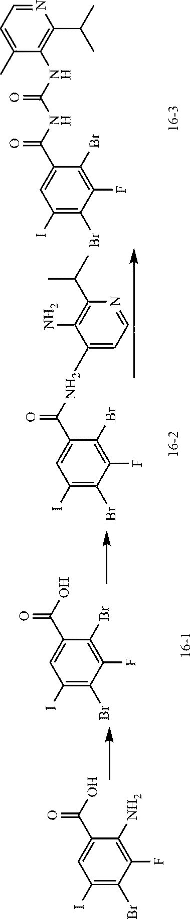

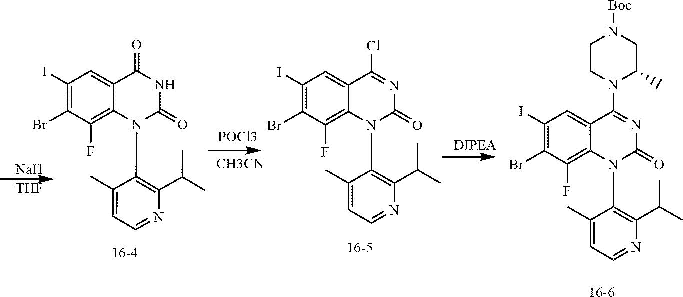

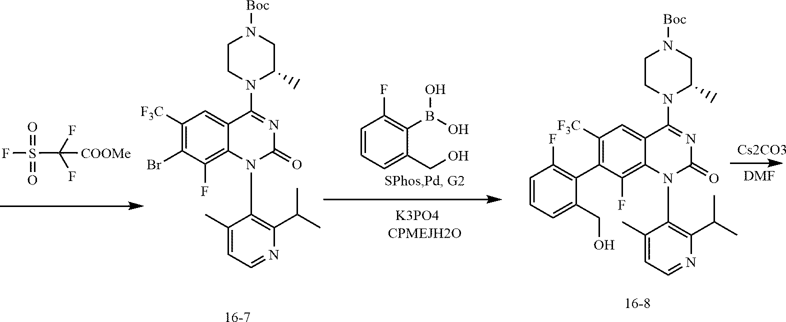

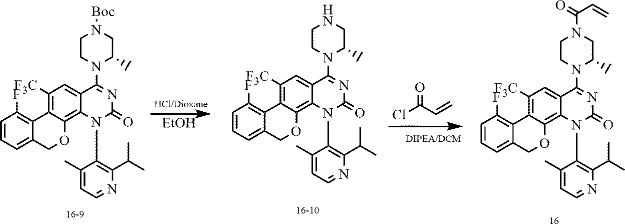

### Step 1: Synthesis of Compound 16-1

2-aminobromo-3-fluoro-5-iodo-benzoic acid (14.0 g, 38.90 mmol), (CH₃CN (230 mL and CuBr (6.70 g, 46.68 mmol) were added in a 500 ml three-necked flask, stir to dissolve, replaced and protected by nitrogen, cooled to 0° C., a solution of tert-butyl nitrite (6.02 g, 58.35 mmol) in CH₃CN (230 mL) was added dropwise,, about 30 minutes drip, rise to 25° C. of reaction 3 hours. The reaction solution was slowly poured into 250 ml of cold saturated NH₄Cl solution, stirred, and 200 ml of EA was added when the exotherm stopped. Stir, let stand, and separate the layers; the aqueous phase is then extracted with 100 ml of EA; the organic phases are combined and washed with 100 ml of saturated NaCl solution; dried over sodium sulfate, filtered, and desolvated to obtain the crude product. Pass through the column (EA:PE=1:1.5) to obtain yellow solid 16-1 (9.3 g, 21.94 mmol, 56.42% yield). ESI-MS m/z: 422.8 [M+H]⁺.

### Step 2: Synthesis of Compound 16-2

Compound 16-1 (6.3 g, 14.87 mmol) and DCM (60 mL) were added to a 100 ml flask, stir to dissolve, add DMF (54.33 mg, 743.28 umol, 57.55 uL), cool in an ice-water bath, add dropwise oxalyl chloride (5.66 g, 44.60 mmol), stirred at room temperature for 30 min, desolvated under reduced pressure, and dissolved with 10 ml of DCM * 2 times with oxalic acid chloride, and dissolved with 10 ml of THF. The above THF solution was added dropwise to NH₃.H₂O (15 M, 277.49 mL), and the reaction was carried out at 25° C. for 2 hours after the dropping. Add 80 ml of EA, 50 ml of saturated brine, stir and separate the layers; extract the aqueous phase with 40 ml of EA again, combine the organic phases, wash with 50 ml of saturated brine; dry over sodium sulfate, filter, and remove the solvent to obtain the crude product. Pass through the column (PE:EA=1:1) to obtain off-white solid product 16-2 (5.8 g, 13.72 mmol, 92.28% yield) . ESI-MS m/z: 421.8 [M+H]⁺.

### Step 3: Synthesis of Compound 16-3

Compound 16-2 (8.0 g, 18.92 mmol) and THF (150 mL) were added to a 500 ml flask, oxalyl chloride (7.20 g, 56.76 mmol) was added in a water bath, heated to reflux for 2 hours, desolvated under reduced pressure, and 10 ml of DCM was added 2 times to remove oxalyl chloride. DCM (120 mL) and 2-isopropyl-4-methylpyridin-3-amine (2.84 g, 18.92 mmol) were added to react at 25° C. for 2 hours, 45 ml of DCM was added to dilute; washed with saturated ammonium chloride solution, washed with saturated aqueous sodium bicarbonate solution, and washed with brine; dry with sodium sulfate, filter, and dissolve. Pass through silica gel column (EA:PE=1:2) to obtain off-white solid 16-3 (4.0 g, 6.68 mmol, 35.29%, yield). ESI-MS m/z: 597.9 [M+H]⁺.

### Step 4: Synthesis of Compound 16-4

Compound 16-3 (4.3 g, 7.18 mmol), THF (60 mL), NaH (1.44 g, 35.89 mmol, 60% purity) were added to a 100 ml flask, replaced and protected by nitrogen, and the reaction was heated to 50° C. for 2 hours. Cooling, <20° C.; the reaction solution was poured into 150 ml of saturated aqueous ammonium chloride solution, 100 ml of EA was added for extraction for 2 times, the organic phases were combined, washed with brine, dried over sodium sulfate, filtered, and desolvated under reduced pressure to obtain the crude product. Slurry with THF:PE=1:5 about 100 ml to obtain off-white solid product 16-4 (3.3 g, 6.37 mmol, 88.73% yield).ESI-MS m/z: 517.9 [M+H]⁺.

### Step 5: Synthesis of Compound 16-5

At room temperature, compound 16-4 (2.1 g, 4.05 mmol), CH₃CN (24 mL), DIPEA (2.10 g, 16.21 mmol, 2.82 mL) were added to a 25 mL flask, and POCl₃ (1.86 g, 12.16 mmol) was added under stirring, heated to 80° C. for 1 hour. Cooling <40° C., degreasing under reduced pressure, DCM(20 ml) was added 2 times for acid removal to obtain yellow-brown solid product 16-5 (4.0 g, crude product).ESI-MS m/z: 535.9 [M+H]⁺.

### Step 6: Synthesis of Compound 16-6

At room temperature, compound 16-5 (4.0 g, 7.45 mmol), DMF (15 mL), tert-butyl(3S)-3-methylpiperazine-1-carboxylate (1.94 g, 9.69 mmol), DIPEA (3.85 g, 29.82 mmol, 5.19 mL) were added in a 25 ml flask reacted at 25° C. for 2 hours. Add 100 ml of EA, 30 ml of water, stir, and separate layers; wash with 10 ml of water for 3 times; wash with brine, dry with sodium sulfate, remove the solvent to obtain crude product; pass the crude product through silica gel column (EA:PE=1:1) to obtain yellow foam solid product 16-6 (4.0 g, 5.71 mmol, 76.61% yield). ESI-MS m/z: 700.1 [M+H]⁺.

### Step 7: Synthesis of Compound 16-7

At room temperature, compound 16-6 (1 g, 1.43 mmol), DMF (18 mL), CuI (67.98 mg, 356.95 umol), 2,2-difluoro-2-fluorosulfonyl acetate methyl (329.16 mg, 1.71 mmol) were added to a 100 ml flask, replaced and protected by nitrogen reacted at 80° C. for 2 hours. Cool down, <30° C. Add 100 ml of EA, 20 ml of water, stir, and separate layers; wash with 20 ml of waterfor 3 times; wash with brine; dry with sodium sulfate, filter, and desolubilize to obtain the crude product. Purification by Pre-HPLC to obtain white solid product 16-7(0.23 g, 357.99 umol, 25.07% yield). ESI-MS m/z: 642.2[M+H]⁺.

### Step 8: Synthesis of Compound 16-8

At room temperature, compound 16-7 (220 mg, 342.42 umol), CPME (3 mL), [2-fluoro-6-(hydroxymethyl) phenyl] boronic acid (116.39 mg, 684.84 umol), K₃PO₄ (218.06 mg, 1.03 mmol), SPhos-Pd-G2 (36.96 mg, 51.36 umol), and H₂O (0.3 mL) were added to a 100 ml flask, replaced and protected by nitrogen, heated to 110° C. and reacted for 0.5 h. Cool to room temperature; separate layers, wash with brine; dry over sodium sulfate, filter; remove solvent to obtain crude product. The crude product was passed through Pre-TLC to obtain a white solid product 16-8 (52 mg, 75.61 umol, 21.23% yield). ESI-MS m/z: 688.3 [M+H]⁺.

### Step 9: Synthesis of Compound 16-9

At room temperature, compound 16-8 (43 mg, 62.53 umol), DMF (5 mL), Cs₂CO₃ (101.69 mg, 312.64 umol) were added to a 10 ml flask, replaced and protected by nitrogen, and the reaction was heated to 65° C. for 2 hours. Cool down; add 30 ml of EA, 5 ml of water, stir, and separate layers; wash with brine 5 ml for 3 times; dry with sodium sulfate, filter, and remove the solvent under reduced pressure to obtain the crude product. The crude product was purified by Pre-TLC (EA:PE=3:1) to obtain a white foamy solid 16-9(35 mg, 52.42 umol, 83.83% yield).ESI-MS m/z: 668.3 [M+H]⁺.

### Step 10: Synthesis of Compounds 16-10

Compound 16-9 (35 mg, 52.42 umol), EtOH (1 mL), HC1/dioxane (2 M, 1.31 mL) were added to a 10 ml flask, and the reaction was carried out at 20° C. for 2 hours. The solvent was removed under reduced pressure to obtain a brown oily liquid 16-10 (32 mg, crude) ESI-MS m/z: 568.2 [M+H]⁺.

### Step 11: Synthesis of Compound 16

Compound 16-10 (32 mg, 56.38 umol), DCM (1 mL), DIPEA (7.29 mg, 56.38 umol, 9.82 uL) were added to a 10 ml flask, replaced and protected by nitrogen, cool to 0° C., add acryloyl chloride (10.21 mg, 112.76 umol), continue to react at 0° C. for 30 min. Add 10 ml of DCM, wash with 10 ml of brine; dry over sodium sulfate, filter; remove the solvent under reduced pressure to obtain crude product. The crude product was passed through Pre-HPLC to obtain product 16 as a white solid (7.6 mg, 12.23 umol, 21.69(% yield). The characteristic peaks of this compound are as follows:NMR (500 MHz, ) δ 8.51 (d, J= 4.9 Hz), 7.75 (d, J= 12.3 Hz), 7.45-7.34 (m), 7.12 (dd, J = 24.0, 15.2 Hz), 6.94 (d, J = 7.4 Hz), 6.74-6.50 (m), 6.40 (d, J = 16.8 Hz), 5.80 (d, J = 10.5 Hz). ESI-MS m/z: 622.2[M+H]⁺.

### Example 17: Synthesis of Compound (S)-8-(4-Acryloyl-2-Methylpiperazin-1-yl)-1,2,12-Trifluoro-11-(2-Isopropyl-4 -Methylpyridin-3-yl)-5H Isochromene[3,4-g] Quinazolin-10(11H)-One

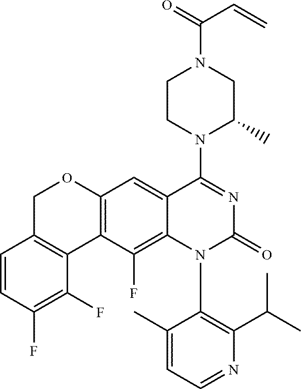 [SMILES: C=CC(=O)N1CCN(c2nc(=O)n(-c3c(C)ccnc3C(C)C)c3c(F)c4c(cc23)OCc2ccc(F)c(F)c2-4)[C@@H](C)C1]

The specific synthesis steps of compound 17 refer to Example 3.ESI-MS m/z: 590.2 [M+H]⁺.

### Example 18: Synthesis of Compound (S)-1-(4-Acryloyl-2-Methylpiperazin-1-yl)-11-Chloro-9,10-Difluoro-4-(2-Isopropyl-4-Methylpyridin-3-yl)-4H-Isochromene[4,3-h] Quinazolin-3(6H)-One

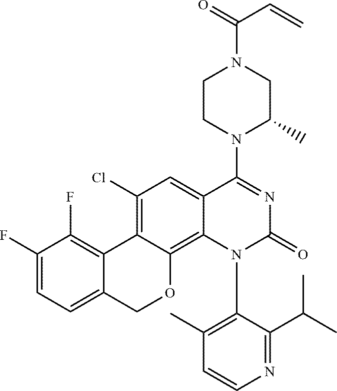 [SMILES: C=CC(=O)N1CCN(c2nc(=O)n(-c3c(C)ccnc3C(C)C)c3c4c(c(Cl)cc23)-c2c(ccc(F)c2F)CO4)[C@@H](C)C1]

For the specific synthesis steps of compound 18, refer to Example 13. The characteristic peaks of this compound are as follows: ¹H NMR (500 MHz, CDCl₃) δ 8.49 (d, J = 4.7 Hz, 1 H), 7.53 (s, 1 H), 7.23 (dd, J = 16.6, 8.4 Hz, 1 H), 7.07 (s, 1 H), 6.90 (dd, J = 7.6, 3.9 Hz, 1 H), 6.77-6.50 (m, 1 H), 6.40 (d, J= 16.7 Hz, 1 H), 5.80 (d, J= 10.5 Hz, 1 H). ESI-MS m/z: 606.2 [M+H]⁺.

### Example 19: Synthesis of Compound 10-((S)-4-Acryloyl-2-Methylpiperazin-1-yl)-8-Chloro-7-Fluoro-13-(2-Isopropyl-4-Methylpyridin-3-yl)-2,3-Dihydrobenzo[4,5]oxa[3,2-h]Quinazo1in-12(13H)-One

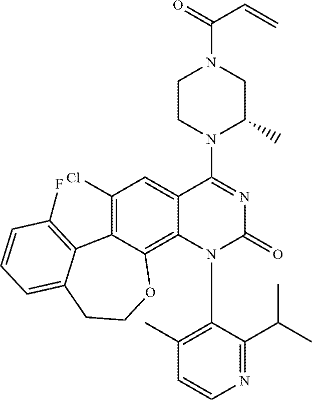 [SMILES: C=CC(=O)N1CCN(c2nc(=O)n(-c3c(C)ccnc3C(C)C)c3c4c(c(Cl)cc23)-c2c(F)cccc2CCO4)[C@@H](C)C1]

For the specific synthesis steps of compound 19, refer to Example 3 and Example 13.¹H NMR (500 MHz, CDCl₃) δ 8.44 (d, J = 4.9 Hz, 1 H), 7.67 (s, 1 H), 7.36 (dd, J = 13.3, 7.9 Hz, 1 H), 7.09 (dd, J= 10.9, 7.2 Hz, 2 H), 7.00 (d, J = 7.5 Hz, 1 H), 6.61 (d, J = 21.4 Hz, 1 H), 6.40 (d, J= 16.9 Hz, 1 H), 5.80 (d, J= 10.5 Hz, 1 H); ESI-MS m/z: 602.2 [M+H]⁺.

### Example 20: Synthesis of Compound (S)-7-(4-Acryloyl-2-Methylpiperazin-1-yl)-5-Chloro-10-(2-Isopropyl-4-Methylpyridine-3-yl)-10H Pyridin[3’,2’:4,5] Pyrano[3,2-h]Quinazolin-9(12H)-One

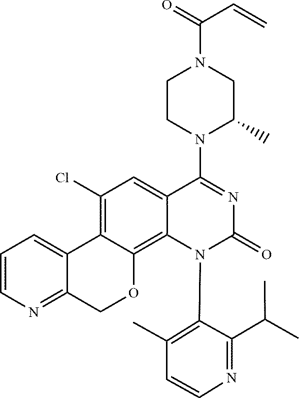 [SMILES: C=CC(=O)N1CCN(c2nc(=O)n(-c3c(C)ccnc3C(C)C)c3c4c(c(Cl)cc23)-c2cccnc2CO4)[C@@H](C)C1]

For the specific synthesis steps of compound 20, refer to Example 13. The characteristic peaks of this compound are as follows:¹H NMR (500 MHz, CDCl₃) δ 8.65 (d, J = 8.0 Hz, 1 H), 8.50 (d, J = 4.9 Hz, 2 H), 7.51 (s, 1 H), 7.35 (dd, J = 8.1, 4.8 Hz, 1 H), 7.07 (dd,J = 10.0, 6.1 Hz, 1 H), 6.58 (s, 1 H), 6.40 (d, J= 16.6 Hz, 1 H), 5.80 (d, J= 10.5 Hz, 1 H). ESI-MS m/z: 571.2[M+H]⁺.

### Example 21: Synthesis of Compound (S)-1-(4-Acryloyl-2-Methylpiperazin-1-yl)-13-Fluoro-4-(2-Isopropyl-4-Methylpyridine-3-yl)-4H-Benzo[5,6] Isochromene[4,3-h]Quinazolin-3(6H)-one

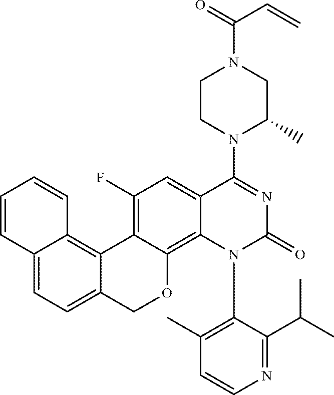 [SMILES: C=CC(=O)N1CCN(c2nc(=O)n(-c3c(C)ccnc3C(C)C)c3c4c(c(F)cc23)-c2c(ccc3ccccc23)CO4)[C@@H](C)C1]

The specific synthesis steps of compound 21 refer to Example 3.¹H NMR (500 MHz, CDCl₃) δ 8.49 (d, J = 4.9 Hz, 1 H), 7.97-7.84 (m, 3 H), 7.57 (ddd, J = 14.9, 13.8, 6.8 Hz, 2 H), 7.30 (d, J = 10.3 Hz, 1 H), 7.21 (d, J = 8.3 Hz, 1 H), 7.06 (d, J = 10.1 Hz, 1 H), 6.62 (dd, J = 34.9, 10.8 Hz, 1 H), 6.41 (d,J= 16.6 Hz, 1 H), 5.81 (dd,J= 10.5, 1.4 Hz, 1 H), 5.30 (s, 6 H), 4.62 (q, J = 13.1 Hz, 2 H); ESI-MS m/z: 604.3 [M+H]⁺.

### Example 22: Synthesis of Compound (S)-10-(4-Acryloyl-2-Methylpiperazin-1-yl)-14-Fluoro-13-(2-Isopropyl-4-Methylpyridine-3-yl)-7H-Benzo[5,6] Isochromene[3,4-g]Quinazolin-12(13H)-One

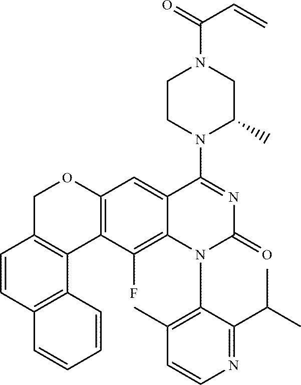 [SMILES: C=CC(=O)N1CCN(c2nc(=O)n(-c3c(C)ccnc3C(C)C)c3c(F)c4c(cc23)OCc2ccc3ccccc3c2-4)[C@@H](C)C1]

For the specific synthesis steps of compound 22, refer to Example 3.ESI-MS m/z: 604.3[M+H]⁺.

### Example 23: Synthesis of Compound (S)-1-(4-Acryloyl-2-Methylpiperazin-1-yl)-11-Chloro-4-(2-Isopropyl-4-Methylpyridine-3-yl)-4H-Pyridin[3’,4’:4,5]Pyrano[3.2-h]Quinazolin-3(6H)-One

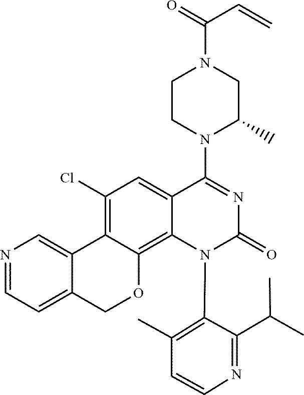 [SMILES: C=CC(=O)N1CCN(c2nc(=O)n(-c3c(C)ccnc3C(C)C)c3c4c(c(Cl)cc23)-c2cnccc2CO4)[C@@H](C)C1]

For the specific synthesis steps of compound 23, refer to Example 13. The characteristic peaks of this compound are as follows:¹H NMR (500 MHz, CDCl₃) δ 9.58 (s, 1 H), 8.59 (d, J = 4.8 Hz, 1 H), 8.50 (d, J = 4.9 Hz, 1 H), 7.54 (s, 1 H), 7.10-7.01 (m, 2 H), 6.58 (s, 1 H), 6.40 (d, J = 17.0 Hz, 1 H), 5.80 (d,J = 10.5 Hz, 1 H). ESI-MS m/z: 571.2 [M+H]⁺.

### Example 24: Synthesis of Compound 10-((S)-4-Acryloyl-2-Methylpiperazin-1-yl)-8-Chloro-13)-(4,6-Diethylpyrimidin-5-yl)-7-Fluoro-2,3 -Dihydrobenzo[4,5] Epoxyheptane[3,2-h]Quinazolin-12(13H)-One

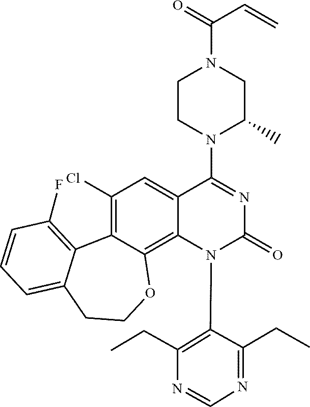 [SMILES: C=CC(=O)N1CCN(c2nc(=O)n(-c3c(CC)ncnc3CC)c3c4c(c(Cl)cc23)-c2c(F)cccc2CCO4)[C@@H](C)C1]

For the specific synthesis steps of compound 24, refer to Example 13. The characteristic peaks of this compound are as follows:¹H NMR (500 MHz, CDCl₃) δ 9.06 (d, J= 12.0 Hz, 1 H), 7.39 (dd, J = 12.9, 7.3 Hz, 1 H), 7.15-6.98 (m, 2 H), 6.67-6.47 (m, 1 H), 6.38 (dd, J = 21.9, 16.6 Hz, 1 H), 5.81 (d, J= 10.5 Hz, 1 H). ESI-MS m/z: 603.2 [M+H]⁺.

### Example 25: Synthesis of Compound 2-(1-Acryloyl-4-(10,11-Difluoro-4-(2-Isopropyl-4-Methylpyridin-3-yl)-3-Oxo-3,6-Dihydro-4H-Isochromene[4,3-h] Quinazolin-1-yl) Piperazin-2-yl)Acetonitrile

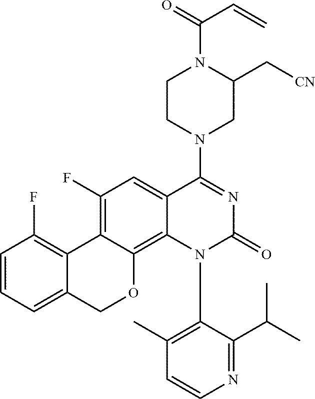 [SMILES: C=CC(=O)N1CCN(c2nc(=O)n(-c3c(C)ccnc3C(C)C)c3c4c(c(F)cc23)-c2c(F)cccc2CO4)CC1CC#N]

For the specific synthesis steps of compound 25, refer to Example 3.¹H NMR (500 MHz, CDCl₃) δ 8.54 (d, J = 4.6 Hz, 1 H), 7.39 (dd, J = 12.6, 7.7 Hz, 1 H), 7.33-7.28 (m, 1 H), 7.16 (dd, J = 20.3, 11.3 Hz, 2 H), 6.93 (d, J = 7.3 Hz, 1 H), 6.68-6.57 (m, 1 H), 6.47-6.39 (m, 1 H), 5.87 (d, J = 10.4 Hz, 1 H), 4.56-4.36 (m, 4 H); ESI-MS m/z: 597.2 [M+H]⁺.

### Example 26: Synthesis of Compound 2-(1-Acryloyl-4-(1,12-Difluoro-11-(2-Isopropyl-4-Methylpyridin-3-yl)-10-Oxo-10,11-Dihydro-5H -Isochromene[3,4-g]Quinazolin-8-yl)Piperazin-2-yl)Acetonitrile

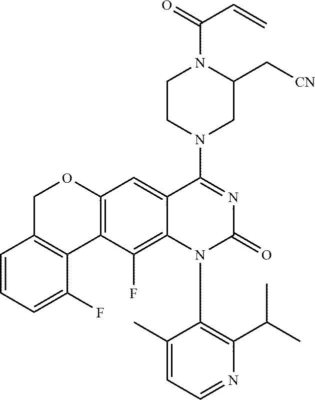 [SMILES: C=CC(=O)N1CCN(c2nc(=O)n(-c3c(C)ccnc3C(C)C)c3c(F)c4c(cc23)OCc2cccc(F)c2-4)CC1CC#N]

The specific synthetic steps of compound 26 refer to Example 3.ESI-MS m/z: 597.2[M+H]⁺.

### Example 27: Synthesis of Compound (S)-1-(4-Acryloyl-2-Methylpiperazin-1-yl)-11-Chloro-9,10-Difluoro-4-(3-Isopropyl) -6,7-Dihydro-5H-Cyclopentyl[c]Pyridin-4-yl)-4H-Isochromo[4,3-h] Quinazolin-3(6H)-one

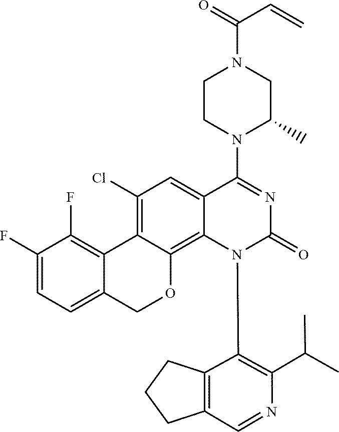 [SMILES: C=CC(=O)N1CCN(c2nc(=O)n(-c3c(C(C)C)ncc4c3CCC4)c3c4c(c(Cl)cc23)-c2c(ccc(F)c2F)CO4)[C@@H](C)C1]

For the specific synthesis steps of compound 27, refer to Example 13.ESI-MS m/z: 632.2[M+H]⁺.

### Example 28: Synthesis of Compound (S)-1-(4-Acryloyl-2-Methylpiperazin-1-yl)-11-Chloro-10-Fluoro-7-Hydroxy-4-(2-Isopropyl) yl-4-Methylpyridin-3-y1)-4H-Isochromo[4,3-h] Quinazolin-3(6H)-One

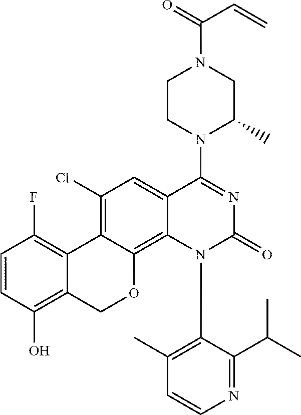 [SMILES: C=CC(=O)N1CCN(c2nc(=O)n(-c3c(C)ccnc3C(C)C)c3c4c(c(Cl)cc23)-c2c(F)ccc(O)c2CO4)[C@@H](C)C1]

For the specific synthesis steps of compound 28, refer to Example 3. ESI-MS m/z: 604.2[M+H]⁺.

### Example 29: Synthesis of Compound (S)-1-(4-Acryloyl-2-Methylpiperazin-1-yl)-11-Chloro-7,10-Difluoro-4-(2-Isopropyl) -4-Methylpyridin-3-yl)-4H-Isochromo[4,3-h] Quinazolin-3(6H)-One

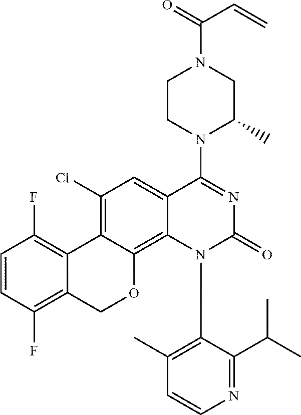 [SMILES: C=CC(=O)N1CCN(c2nc(=O)n(-c3c(C)ccnc3C(C)C)c3c4c(c(Cl)cc23)-c2c(F)ccc(F)c2CO4)[C@@H](C)C1]

For the specific synthesis steps of compound 29, refer to Example 3.ESI-MS m/z: 606.2[M+H]⁺.

### Example 30: Synthesis of Compound 10-((S)-4-Acryloyl-2-Methylpiperazin-1-yl)-8-Chloro-6,7-Difluoro-13-(2-Isopropyl-4-Methylpyrid in-3-yl)-2,3-Dihydrobenzo[4,5] Oxa[3,2-h]Quinazolin-12(13H)-One

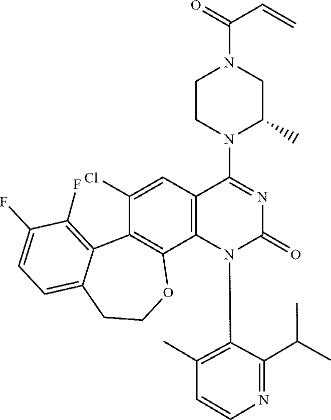 [SMILES: C=CC(=O)N1CCN(c2nc(=O)n(-c3c(C)ccnc3C(C)C)c3c4c(c(Cl)cc23)-c2c(ccc(F)c2F)CCO4)[C@@H](C)C1]

For the specific synthesis steps of compound 30, refer to Example 3.ESI-MS m/z: 620.2[M+H]⁺.

### Example 31: Synthesis of Compound 10-((S)-4-Actyloyl-2-Methylpiperazin-1-yl)-8-Chloro-13-(3,5-Diethylpyridin-4-yl)-7-Fluoro-2,3-Dihydrobenzo[4,5]Oxa[3,2-h]Quinazolin-12(13H)-One

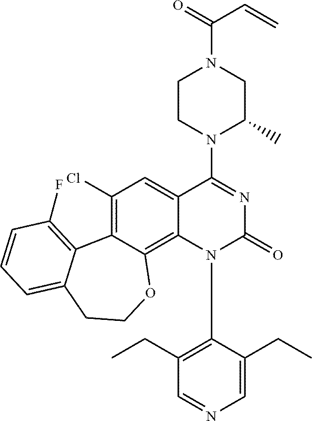 [SMILES: C=CC(=O)N1CCN(c2nc(=O)n(-c3c(CC)cncc3CC)c3c4c(c(Cl)cc23)-c2c(F)cccc2CCO4)[C@@H](C)C1]

For the specific synthesis steps of compound 31, refer to Example 13.ESI-MS m/z: 602.2[M+H]⁺.

### Example 32: Synthesis of Compound 10-((S)-4-Acryloyl-2-Methylpiperazin-1-yl)-8-Chloro-13-(4,6-Dicyclopropylpyrimidine-5-Base)-7 -Fluoro-2,3-Dihydrobenzo[4,5] Oxa[3,2-h]Quinazolin-12(13H)-One

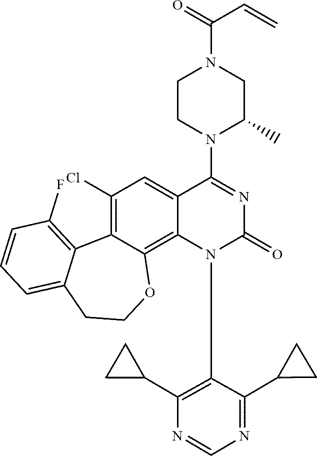 [SMILES: C=CC(=O)N1CCN(c2nc(=O)n(-c3c(C4CC4)ncnc3C3CC3)c3c4c(c(Cl)cc23)-c2c(F)cccc2CCO4)[C@@H](C)C1]

For the specific synthesis steps of compound 32, refer to Example 13. ESI-MS m/z: 627.2[M+H]⁺.

### Example 33: Synthesis of Compound 1-(5-Acryloyl-2,5-Diazabicyclo [4.1.0] Heptan-2-yl)-11-Chloro-10-Fluoro-4-(2-Isopropyl-4-Methylpyridin-3-yl)-4H-Isochromeno[4,3-h]Quinazolin-3(6H)-One

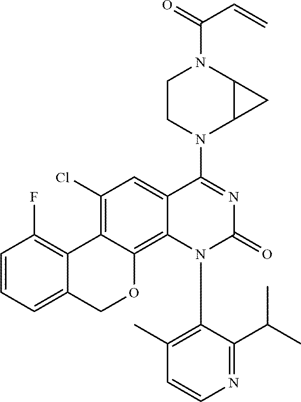 [SMILES: C=CC(=O)N1CCN(c2nc(=O)n(-c3c(C)ccnc3C(C)C)c3c4c(c(Cl)cc23)-c2c(F)cccc2CO4)C2CC21]

For the specific synthesis steps of compound 33, refer to the synthesis of intermediate M-11 and Example 3. ESI-MS m/z: 586.2[M+H]⁺.

### Example 34: Synthesis of Compound (S)-11-Chloro-9,10-Difluoro-1-(4-(2-Fluoroacryloyl)-2-Methylpiperazin-1-yl)-4-( 2-Isopropyl-4-Methylpyridin-3-yl)-4H-Isochromo[4,3-h] Quinazolin-3(6H)-One

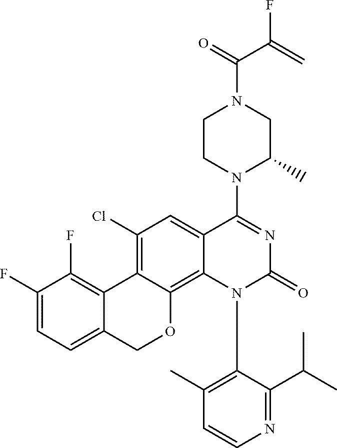 [SMILES: C=C(F)C(=O)N1CCN(c2nc(=O)n(-c3c(C)ccnc3C(C)C)c3c4c(c(Cl)cc23)-c2c(ccc(F)c2F)CO4)[C@@H](C)C1]

The specific synthesis steps of compound 34 refer to Example 3.ESI-MS m/z: 624.2[M+H]⁺.

Examples compounds 17-23 and 28-30 were synthesized with reference to the steps of Example compound 3, the carboxylic acid was reduced to an aldehyde group and then reduced to a hydroxyl group, and then the bromine atom was subjected to boron esterification. Using the common intermediate M-11 or 13-6 and the corresponding boron ester through Suzuki coupling, ring closure, deprotection and introduction of acryloyl group to finally obtain the target product .Referring to the synthesis of M-11, Example compounds 24, 27, 31 and 32 were respectively ethylated, isopropylated and cyclopropylated with chlorine, and then coupled with the corresponding boron ester through Suzuki, closed ring, deprotected, and introduce the acryloyl group to finally obtain the target product. After Boc protection of Example compounds 25 and 26, referring to the synthesis of M-11, they were coupled with the corresponding boron ester by Suzuki, ring closure, deprotection and introduction of acryloyl groups to finally obtain the target product.

The corresponding synthetic intermediates are as follows:

| No. | Intermediate |
|---|---|
| 17, 18 | 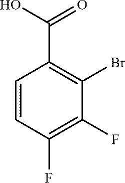 [SMILES: O=C(O)c1ccc(F)c(F)c1Br] |
| 19 | 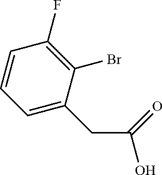 [SMILES: O=C(O)Cc1cccc(F)c1Br] |
| 20 | 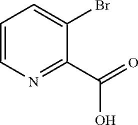 |
| 21, 22 | 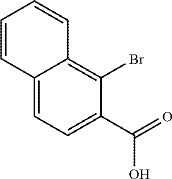 [SMILES: O=C(O)c1ccc2ccccc2c1Br] |
| 23 | 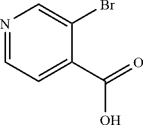 |
| 24, 32 | 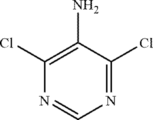 |
| 25, 26 | 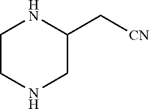 |
| 27 | 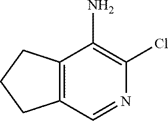 [SMILES: Nc1c(Cl)ncc2c1CCC2] |
| 28 | 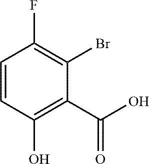 [SMILES: O=C(O)c1c(O)ccc(F)c1Br] |
| 29 | 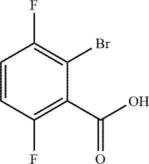 [SMILES: O=C(O)c1c(F)ccc(F)c1Br] |
| 30 | 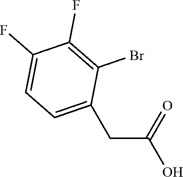 [SMILES: O=C(O)Cc1ccc(F)c(F)c1Br] |
| 31 | 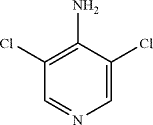 |
| 33 | 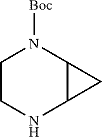 [SMILES: CC(C)(C)OC(=O)N1CCNC2CC21] |
| 34 | 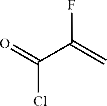 |

### Example 35: Synthesis of Compound (S)-1-(4-Acryloyl-2-Methylpiperazin-1-yl)-11-Chloro-9,10-Difluoro-4-(2-Isopropyl) -4-Methylpyridin-3-yl)-4H-Isochromo[4,3-h] Quinazolin-3(6H)-One-6,6-d2

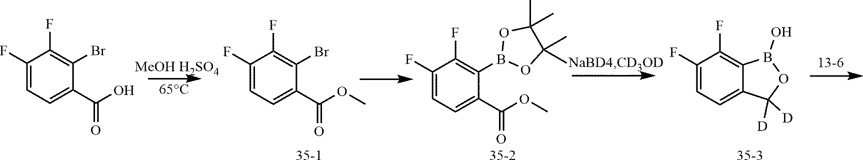

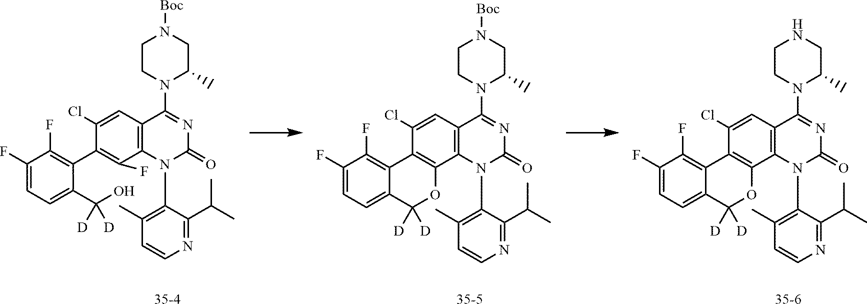

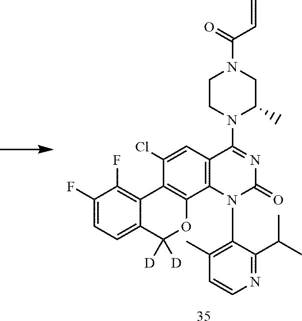

### Step 1: Synthesis of Compound 35-1

At room temperature, 2-bromo-3,4-difluorobenzoic acid (20.00 g, 84.39 mmol) was dissolved in methanol (100.00 mL), concentrated sulfuric acid (4.52 mL) was added, and the temperature was raised to 70° C. for 8 h. The reaction solution was concentrated, and the concentrate was separated and purified by column chromatography to obtain the target product 35-1 (18.2 g, 85.92% yield).ESI-MS m/z: 250.9[M+H]⁺.

### Step 2: Synthesis of Compound 35-2

At room temperature, compound 35-1 (8.00 g, 31.87 mmol), pinacol diboronic acid (9.60 g), PdCl₂(dppf).CH₂Cl₂ and potassium acetate were dissolved in dioxane, replaced with nitrogen, and the temperature was raised to 100° C. react overnight. After the reaction mixture was filtered, ethyl acetate and water were added thereto for extraction, the organic phase was dried over anhydrous sodium sulfate and then concentrated, and the concentrate was purified by silica gel column to obtain the target product 35-2.ESI-MS m/z: 299.1 [M+H]⁺.

### Step 3: Synthesis of Compound 35-3

At room temperature, compound 35-2 (1.72 g, 5.77 mmol) was dissolved in tetrahydrofuran (8.60 mL), NaBD₄ (0.8600 g) was added at 25° C., and CD₃OD (8.60 mL) was slowly added dropwise. After the addition was completed, the mixture was stirred for 30 min. The reaction solution was evaporated to dryness, the evaporated crude product was dissolved in water, and the pH was adjusted to acid 3 with dilute hydrochloric acid, a white solid was precipitated, suction filtered, and the filter cake was the target product 35-3 (0.8700 g, 87.69% yield).ESI-MS m/z: 173.1[M+H]⁺.

### Step 4: Synthesis of Compound 35-4

Compound 35-3 (0.71 g, 4.11 mmol), compound 13-6 (1.00 g, 1.64 mmol), sphos G2 Pd (0.12 g) and potassium phosphate (1.05 g) were dissolved in a mixed solution of cyclopentyl methyl ether (9.00 mL) and water (1.00 mL) at room temperature, nitrogen was replaced, and the temperature was heated to 100° C. by microwave for 25 min. The reaction solution was extracted three times with EA, washed with saturated brine, dried over anhydrous magnesium sulfate, evaporated to dryness, and separated and purified by column chromatography to obtain the crude product. The crude product was separated and purified by thin-layer plate to obtain the target product 35-4 (0.11 g, 9.93% yield).ESI-MS m/z: 674.3[M+H]⁺.

The synthesis steps from step 5 to step 7 refer to Example 1.ESI-MS m/z: 608.2[M+H]⁺.

### Example 36: Synthesis of Compound (S)-8-(4-Acryloyl-2-Methylpiperazin-1-yl)-11-(2-Isopropyl-4-Methylpyridin-3-yl) -5H-Isochromene[3’,4′:5,6]Pyrido[2,3-d]Pyrimidin-10(11H)-One

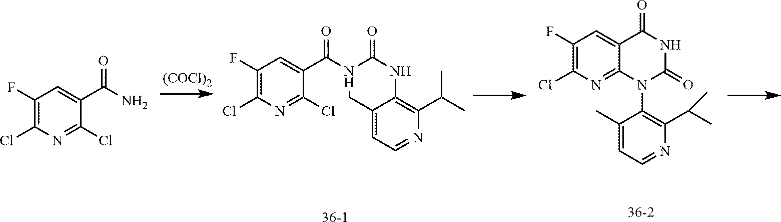

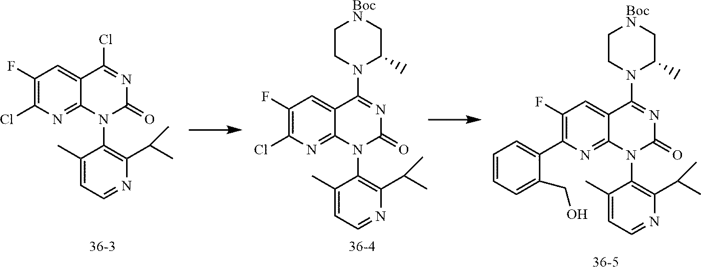

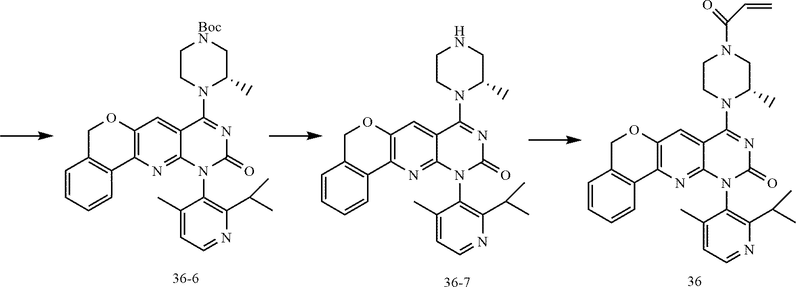

The specific synthesis steps of step 1 and step 2 refer to Example 16.

### Step 3: Synthesis of Compound 36-3

Compound 36-2 (2 g, 5.73 mmol), ACN (40 mL), DIEA (3.71 g, 28.67 mmol, 4.99 mL) were added to a 250 ml single-necked flask, then POCl₃ (2.64 g, 17.20 mmol) was added dropwise at 0° C., heated to 80° C. and stirred for one hour. Direct concentration to obtain black solid crude product 36-3 (1.91 g, 5.20 mmol, 90.70% yield).

### Step 4: Synthesis of Compound 36-4

Compound 36-3 (1.9 g, 5.17 mmol), DCM (40 mL), DIEA (2.67 g, 20.70 mmol, 3.60 mL) were added to a 250 ml single-necked flask, and (3S)-3-methylpiperazine-1-carboxylate tert-butyl ester (1.35 g, 6.73 mmol) was added with stirring at 0° C., then returned to room temperature and stirred for 1 hour. Silica gel was added and the sample was mixed and passed through the column (DCM/MEOH = 20:1), and concentrated to obtain the product as a brown solid (2.2 g, 4.14 mmol, 80.07% yield) .

### Step 5: Synthesis of Compound 36-5

(Hydroxymethyl) phenyl] boronic acid (114.46 mg, 753.27 umol), compound 36-4 (200 mg, 376.63 umol), SPHOS Pd G2 (54.21 mg, 75.33 umol), K₃PO₄ (159.89 mg, 753.27 umol), CPME (4 mL), H₂O (0.4 mL) were added to a 20 ml microwave tube, replaced with nitrogen, the reaction was carried out by microwave at 90° C. for 50 minutes. EA was added, the organic phase was washed with water, the organic phase was dried over anhydrous sodium sulfate, filtered and spin-dried. Purified by silica gel column with DCM/MEOH (15:1) to give the product as pale yellow solid (170 mg, 282.07 umol, 74.89% yield) ESI-MS m/z: 603.4[M+H]⁺.

### Step 6: Synthesis of Compound 36-6

Compound 36-5 (150 mg, 248.88 umol), Cs₂CO3 (162.18 mg, 497.76 umol), DMF (3 mL) were added to a microwave tube, and the reaction was carried out at 100° C. for 15 minutes in a microwave. EA was added, the organic phase was washed with water, the organic phase was dried over anhydrous sodium sulfate, filtered and concentrated to obtain a light yellow solid product (130 mg, 223.10 umol, 89.64% yield). ESI-MS m/z: 583.4[M+H]⁺.

### Step 7: Synthesis of Step 7: Compound 36-7

Compound 36-6 (130 mg, 223.10 umol) and DCM (6 mL) were added to a 25 ml single-necked flask, TFA (1 mL) was added under stirring at room temperature, and then stirred at room temperature for 10 minutes. Concentrated directly to give pale yellow oily product (97 mg, 201.00 umol, 90.10% yield). ESI-MS m/z: 483.4[M+H]⁺.

### Step 8: Synthesis of Compound 36

Compound 36-7 (97 mg, 201.00 umol), DCM (5 mL), DIEA (77.94 mg, 603.01 umol, 105.03 uL) were added to a 25 ml single-necked bottle, and acryloyl chloride (18.19 mg, 201.00 umol) was slowly added dropwise at 0° C., then stir for 10 minutes. Add ice water, extract with EA, wash the organic phase with water, dry the organic phase with anhydrous sodium sulfate, filter and spin dry. Purification by Pre-HPLC to obtain the title product as a pale yellow solid (58.8 mg, 109.57 umol, 54.51 % yield). The characteristic peaks of this compound are as follows: ¹H NMR (500 MHz, CDCl₃) δ 8.60 (d, J = 4.9 Hz, 1 H), 7.57-7.47 (m, 2 H), 7.41 (t, J = 7.5 Hz, 1 H), 7.29 (t, J= 7.8 Hz, 1 H), 7.14 (dd, J= 14.5, 6.1 Hz, 2 H), 6.68-6.53 (m, 1 H), 6.40 (d, J= 16.9 Hz, 1 H), 5.80 (d, J= 10.6 Hz, 1 H). ESI-MS m/z: 537.4[M+H]⁺.

### Example 37: Synthesis of Compound (S)-8-(4-Acryloyl-2-Methylpiperazin-1-yl)-1-Hydroxy-11-(2-Isopropyl-4-Methylpyridine-3-yl)-5 H-Isochromene [3’,4’:5,6] Pyrido [2,3-d] Pyrimidin 10(11H)-One

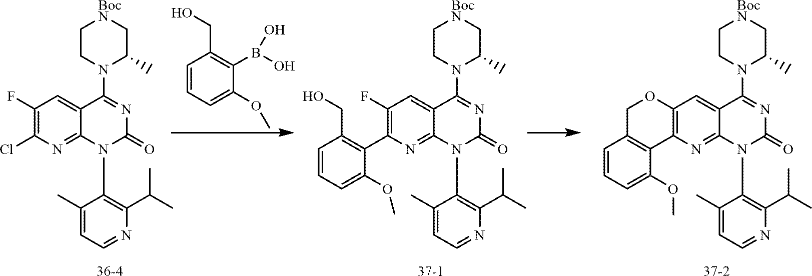

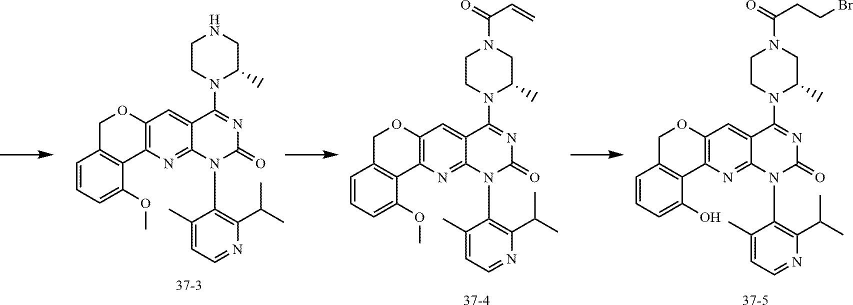

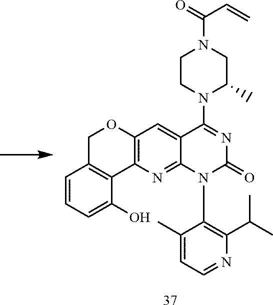

### Step 1: Synthesis of Compound 37-1

(Hydroxymethyl)-6-methoxyphenyl] boronic acid (137.08 mg, 753.27 umol), compound 36-4 (200 mg, 376.63 umol), CPME (3 mL), H₂O , SPHOS Pd G2 (54.21 mg, 75.33 umol), K₃PO₄ (1 59.89 mg, 753.27 umol) were added to a microwave tube (0.5 mL), , after the reaction solution was replaced with nitrogen, the reaction was microwaved at 90° C. for 50 minutes. EA and water were added, dried over anhydrous sodium sulfate, filtered and spin-dried. Purified by Pre-TLC to obtain the target product as a brown solid 37-1(211 mg, 333.48 umol, 88.54% yield). ESI-MS m/z: 633.46[M+H]⁺.

### Step 2: Synthesis of Compound 37-2

Compound 37-1 (210 mg, 331.90 umol), DMF (4 mL), Cs₂CO₃ (324.42 mg, 995.70 umol) were added to a microwave tube, and the reaction was microwaved at 100° C. for 15 minutes. EA was added, the organic phase was washed with water, the organic phase was dried with anhydrous sodium sulfate, filtered, and concentrated to obtain the target product as a pale yellow solid 37-2 (191 mg, 311.73 umol, 93.92% yield). ESI-MS m/z: 613.43[M+H]⁺.

### Step 3: Synthesis of Compound 37-3

Compound 37-2 (190 mg, 310.09 umol), DCM (5 mL), TFA (1 mL) were added to a 25 ml single-necked bottle, and the mixture was stirred at room temperature for 20 minutes. Direct concentration to obtain the target product as a brown oil 37-3 (149 mg, 290.67 umol, 93.74% yield). ESI-MS m/z: 513.35[M+H]⁺.

### Step 4: Synthesis of Compound 37-4

Compound 37-3 (141 mg, 275.07 umol), DCM (5 mL), DIEA (106.65 mg, 825.20 umol, 143.73 uL) were added to a 25 ml single-necked flask, acryloyl chloride (24.90 mg, 275.07 umol) was added under stirring at 0° C. , and the reaction was stirred for five minutes. Add ice water, saturated sodium bicarbonate solution, quench, extract with EA, dry the organic phase over anhydrous sodium sulfate, filter and spin dry. Purified by Pre-TLC to obtain the target product as pale yellow solid 37-4 (211 mg, 333.48 umol, 88.54% yield). ESI-MS m/z: 567.40[M+H]⁺.

### Step 5: Synthesis of Compound 37-5

Compound 37-4 (50 mg, 88.24 umol) and dichloroethane (10 mL) were added to a single-necked flask, BBr₃ (88.24 mg, 352.95 umol) was added dropwise at 0° C., and then the temperature was raised to 60° C. and stirred for 6 hours. Add ice water at 0° C., then add saturated sodium bicarbonate solution, extract with EA, dry, filter, and spin dry. Concentrate directly to obtain the crude target product as pale red solid 37-5. ESI-MS m/z: 633.00[M+H]⁺.

### Step 6: Synthesis of Compound 37

Compound 37-5 (27 mg, 42.62 umol) and THF (9 mL) were added to a single-necked flask, then 1 ml of 2N sodium hydroxide solution was added, and the mixture was stirred for 30 minutes. Saturated sodium bicarbonate solution was added, then THF was removed, extracted with EA, and the organic phase was dried over anhydrous sodium sulfate, filtered, and spin-dried. Purified by Pre-TLC to obtain the target product as a brown solid 37 (13.0 mg, 23.52 umol, 55.20% yield). ESI-MS m/z: 553.3[M+H]⁺.

The following examples were synthesized using the methods described above, or analogously using the corresponding intermediates.

| | | | | |
|---|---|---|---|---|
| 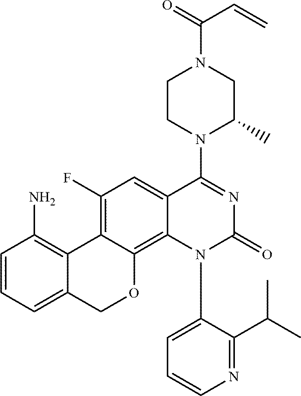 [SMILES: C=CC(=O)N1CCN(c2nc(=O)n(-c3cccnc3C(C)C)c3c4c(c(F)cc23)-c2c(N)cccc2CO4)[C@@H](C)C1] 38 [M+H]⁺: 555.2 | 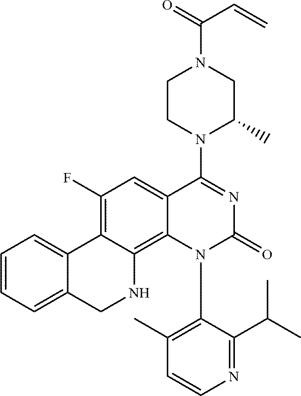 [SMILES: C=CC(=O)N1CCN(c2nc(=O)n(-c3c(C)ccnc3C(C)C)c3c4c(c(F)cc23)-c2ccccc2CN4)[C@@H](C)C1] 39 [M+H]⁺: 553.3 | 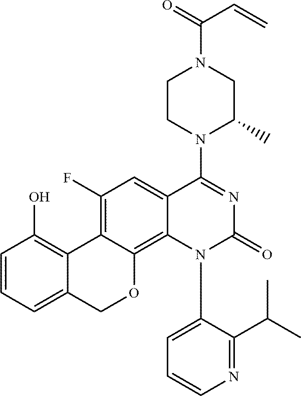 [SMILES: C=CC(=O)N1CCN(c2nc(=O)n(-c3cccnc3C(C)C)c3c4c(c(F)cc23)-c2c(O)cccc2CO4)[C@@H](C)C1] 40 [M+H]⁺: 556.2 | 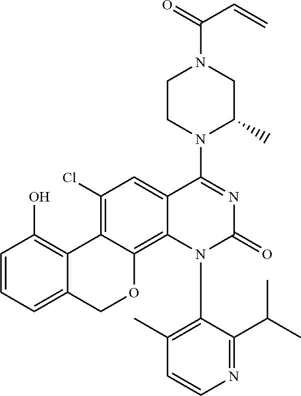 [SMILES: C=CC(=O)N1CCN(c2nc(=O)n(-c3c(C)ccnc3C(C)C)c3c4c(c(Cl)cc23)-c2c(O)cccc2CO4)[C@@H](C)C1] 41 [M+H]⁺: 586.2 | 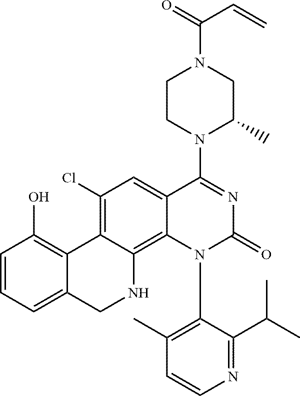 [SMILES: C=CC(=O)N1CCN(c2nc(=O)n(-c3c(C)ccnc3C(C)C)c3c4c(c(Cl)cc23)-c2c(O)cccc2CN4)[C@@H](C)C1] 42 [M+H]⁺: 585.2 |
| 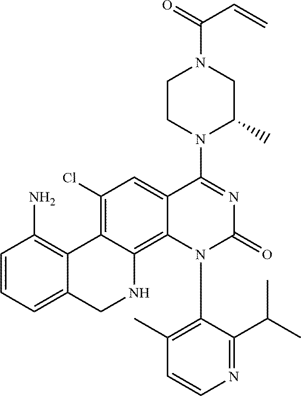 [SMILES: C=CC(=O)N1CCN(c2nc(=O)n(-c3c(C)ccnc3C(C)C)c3c4c(c(Cl)cc23)-c2c(N)cccc2CN4)[C@@H](C)C1] 43 [M+H]⁺: 584.3 | 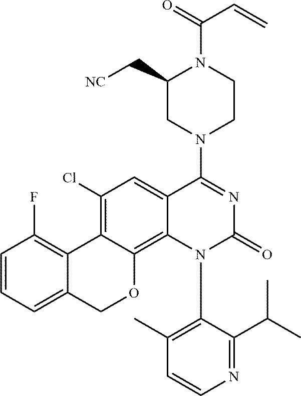 44 [M+H]⁺: 613.2 | 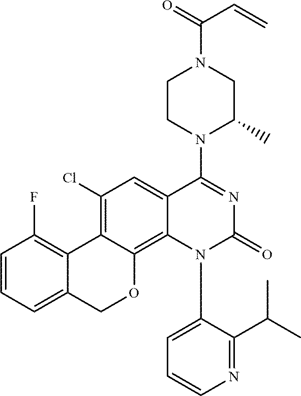 [SMILES: C=CC(=O)N1CCN(c2nc(=O)n(-c3cccnc3C(C)C)c3c4c(c(Cl)cc23)-c2c(F)cccc2CO4)[C@@H](C)C1] 45 [M+H]⁺: 574.2 | 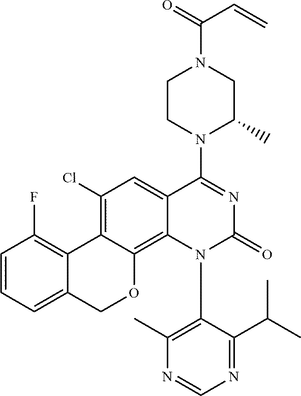 [SMILES: C=CC(=O)N1CCN(c2nc(=O)n(-c3c(C)ncnc3C(C)C)c3c4c(c(Cl)cc23)-c2c(F)cccc2CO4)[C@@H](C)C1] 46 [M+H]⁺: 589.2 | 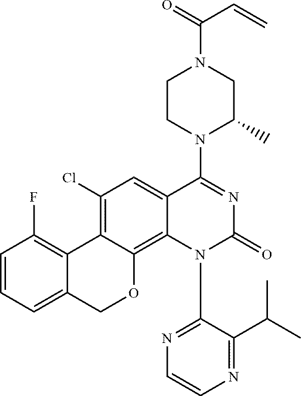 [SMILES: C=CC(=O)N1CCN(c2nc(=O)n(-c3nccnc3C(C)C)c3c4c(c(Cl)cc23)-c2c(F)cccc2CO4)[C@@H](C)C1] 47 [M+H]⁺: 575.2 |
| 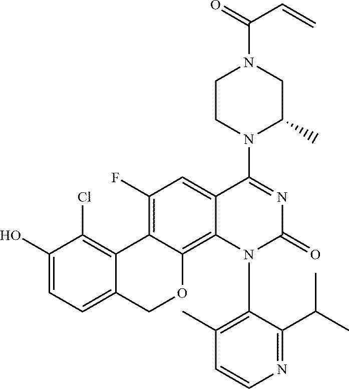 [SMILES: C=CC(=O)N1CCN(c2nc(=O)n(-c3c(C)ccnc3C(C)C)c3c4c(c(F)cc23)-c2c(ccc(O)c2Cl)CO4)[C@@H](C)C1] 48 [M+H]⁺: 604.2 | 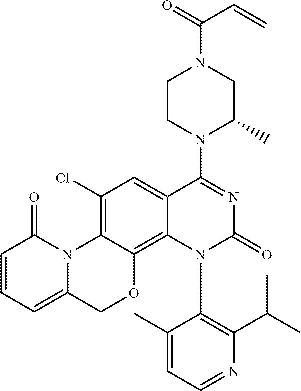 [SMILES: C=CC(=O)N1CCN(c2nc(=O)n(-c3c(C)ccnc3C(C)C)c3c4c(c(Cl)cc23)-n2c(cccc2=O)CO4)[C@@H](C)C1] 49 [M+H]⁺: 587.2 | 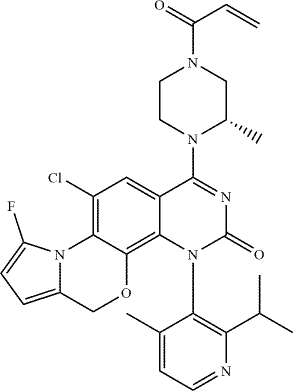 [SMILES: C=CC(=O)N1CCN(c2nc(=O)n(-c3c(C)ccnc3C(C)C)c3c4c(c(Cl)cc23)-n2c(F)ccc2CO4)[C@@H](C)C1] 50 [M+H]⁺: 577.2 | 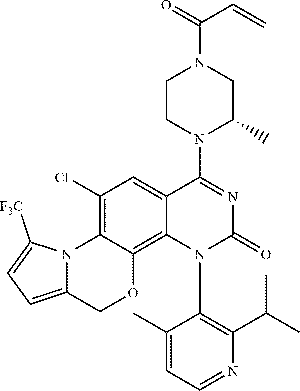 [SMILES: C=CC(=O)N1CCN(c2nc(=O)n(-c3c(C)ccnc3C(C)C)c3c4c(c(Cl)cc23)-n2c(ccc2C(F)(F)F)CO4)[C@@H](C)C1] 51 [M+H]⁺:: 627.2 | 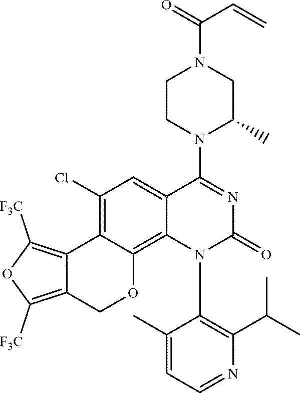 [SMILES: C=CC(=O)N1CCN(c2nc(=O)n(-c3c(C)ccnc3C(C)C)c3c4c(c(Cl)cc23)-c2c(C(F)(F)F)oc(C(F)(F)F)c2CO4)[C@@H](C)C1] 52 [M+H]⁺: 696.3 |
| 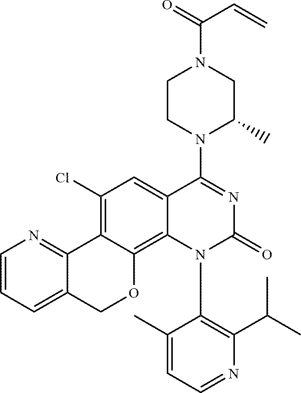 [SMILES: C=CC(=O)N1CCN(c2nc(=O)n(-c3c(C)ccnc3C(C)C)c3c4c(c(Cl)cc23)-c2ncccc2CO4)[C@@H](C)C1] 53 [M+H]⁺: 571.2 | 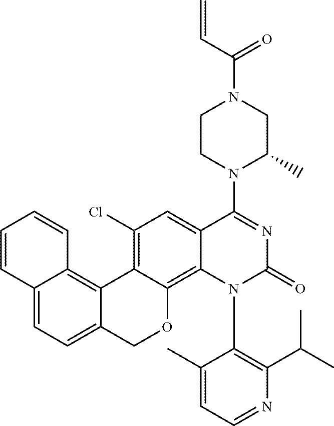 [SMILES: C=CC(=O)N1CCN(c2nc(=O)n(-c3c(C)ccnc3C(C)C)c3c4c(c(Cl)cc23)-c2c(ccc3ccccc23)CO4)[C@@H](C)C1] 54 [M+H]⁺: 620.2 | 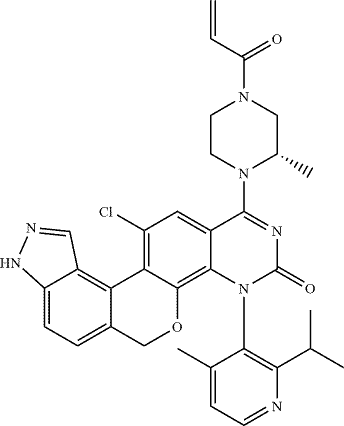 [SMILES: C=CC(=O)N1CCN(c2nc(=O)n(-c3c(C)ccnc3C(C)C)c3c4c(c(Cl)cc23)-c2c(ccc3[nH]ncc23)CO4)[C@@H](C)C1] 55 [M+H]⁺: 610.2 | 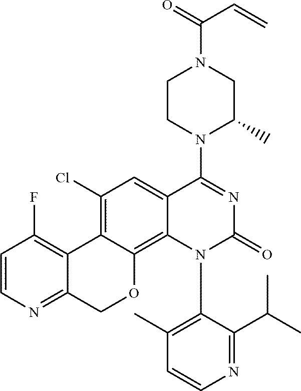 [SMILES: C=CC(=O)N1CCN(c2nc(=O)n(-c3c(C)ccnc3C(C)C)c3c4c(c(Cl)cc23)-c2c(F)ccnc2CO4)[C@@H](C)C1] 56 [M+H]⁺: 589.2 | 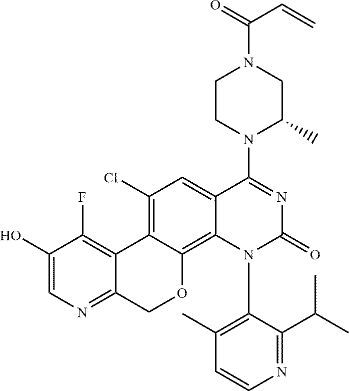 [SMILES: C=CC(=O)N1CCN(c2nc(=O)n(-c3c(C)ccnc3C(C)C)c3c4c(c(Cl)cc23)-c2c(ncc(O)c2F)CO4)[C@@H](C)C1] 57 [M+H]⁺: 605.2 |
| 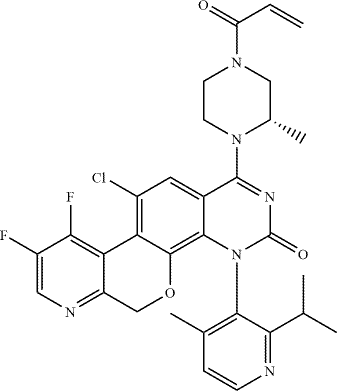 [SMILES: C=CC(=O)N1CCN(c2nc(=O)n(-c3c(C)ccnc3C(C)C)c3c4c(c(Cl)cc23)-c2c(ncc(F)c2F)CO4)[C@@H](C)C1] 58 [M+H]⁺: 607.2 | 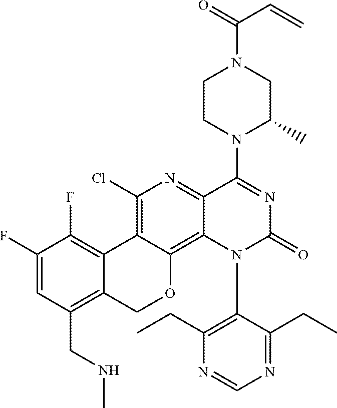 [SMILES: C=CC(=O)N1CCN(c2nc(=O)n(-c3c(CC)ncnc3CC)c3c4c(c(Cl)nc23)-c2c(F)c(F)cc(CNC)c2CO4)[C@@H](C)C1] 59 [M+H]⁺: 651.2 | 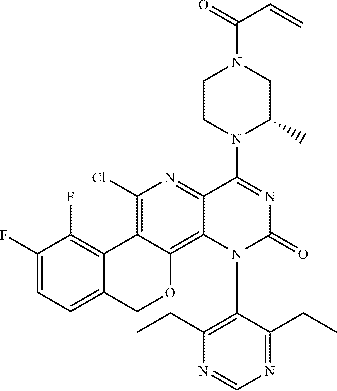 [SMILES: C=CC(=O)N1CCN(c2nc(=O)n(-c3c(CC)ncnc3CC)c3c4c(c(Cl)nc23)-c2c(ccc(F)c2F)CO4)[C@@H](C)C1] 60 [M+H]⁺: 608.2 | 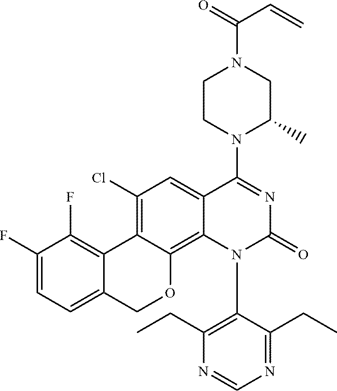 [SMILES: C=CC(=O)N1CCN(c2nc(=O)n(-c3c(CC)ncnc3CC)c3c4c(c(Cl)cc23)-c2c(ccc(F)c2F)CO4)[C@@H](C)C1] 61 [M+H]⁺: 607.2 | 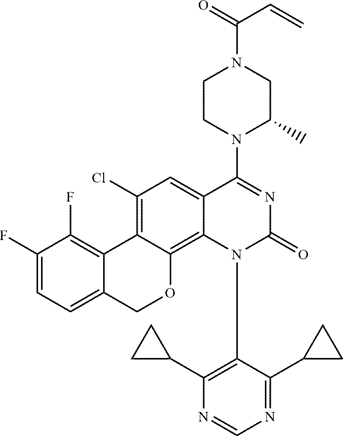 [SMILES: C=CC(=O)N1CCN(c2nc(=O)n(-c3c(C4CC4)ncnc3C3CC3)c3c4c(c(Cl)cc23)-c2c(ccc(F)c2F)CO4)[C@@H](C)C1] 62 [M+H]⁺: 631.2 |
| 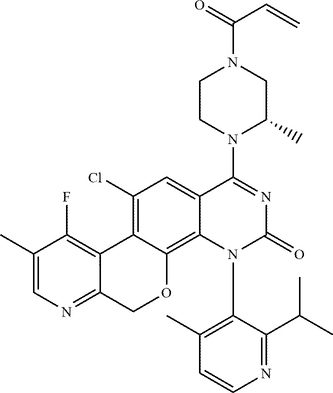 [SMILES: C=CC(=O)N1CCN(c2nc(=O)n(-c3c(C)ccnc3C(C)C)c3c4c(c(Cl)cc23)-c2c(ncc(C)c2F)CO4)[C@@H](C)C1] 63 | 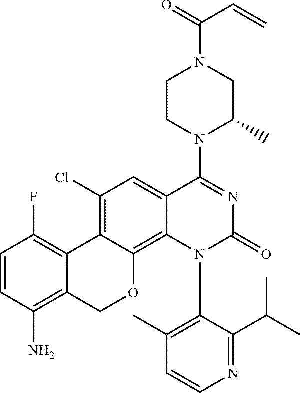 [SMILES: C=CC(=O)N1CCN(c2nc(=O)n(-c3c(C)ccnc3C(C)C)c3c4c(c(Cl)cc23)-c2c(F)ccc(N)c2CO4)[C@@H](C)C1] 64 [M+H]⁺: 603.2 | 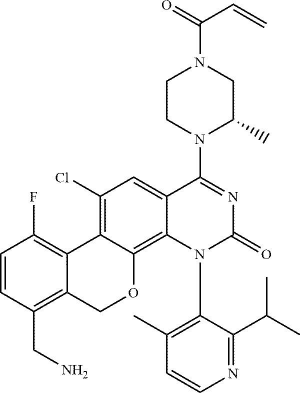 [SMILES: C=CC(=O)N1CCN(c2nc(=O)n(-c3c(C)ccnc3C(C)C)c3c4c(c(Cl)cc23)-c2c(F)ccc(CN)c2CO4)[C@@H](C)C1] 65 [M+H]⁺: 617.2 | 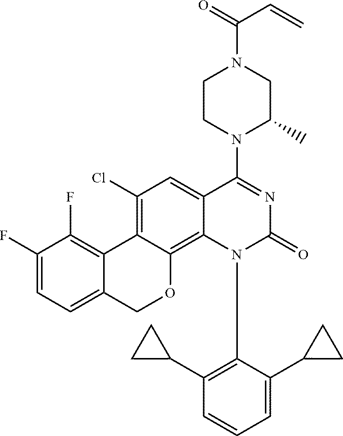 [SMILES: C=CC(=O)N1CCN(c2nc(=O)n(-c3c(C4CC4)cccc3C3CC3)c3c4c(c(Cl)cc23)-c2c(ccc(F)c2F)CO4)[C@@H](C)C1] 66 [M+H]⁺: 629.2 | 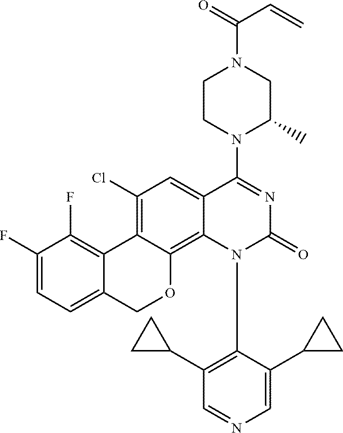 [SMILES: C=CC(=O)N1CCN(c2nc(=O)n(-c3c(C4CC4)cncc3C3CC3)c3c4c(c(Cl)cc23)-c2c(ccc(F)c2F)CO4)[C@@H](C)C1] 67 [M+H]⁺:630.2 |
| [M+H]⁺: 603.2 | | | | |
| 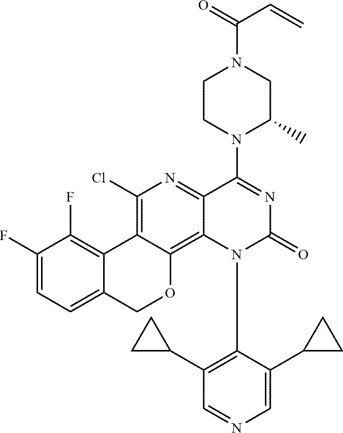 [SMILES: C=CC(=O)N1CCN(c2nc(=O)n(-c3c(C4CC4)cncc3C3CC3)c3c4c(c(Cl)nc23)-c2c(ccc(F)c2F)CO4)[C@@H](C)C1] 68 [M+H]⁺: 631.2 | 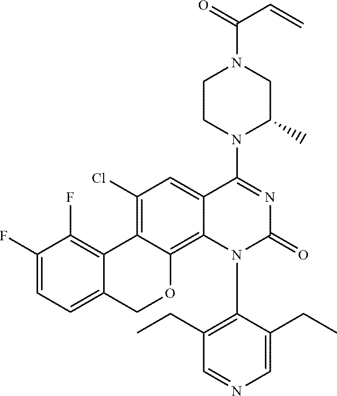 [SMILES: C=CC(=O)N1CCN(c2nc(=O)n(-c3c(CC)cncc3CC)c3c4c(c(Cl)cc23)-c2c(ccc(F)c2F)CO4)[C@@H](C)C1] 69 [M+H]⁺: 606.2 | 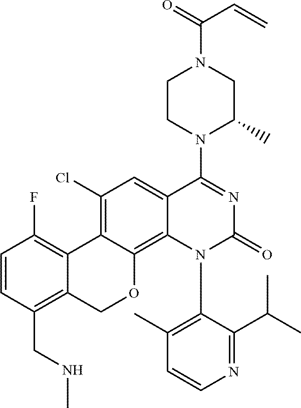 [SMILES: C=CC(=O)N1CCN(c2nc(=O)n(-c3c(C)ccnc3C(C)C)c3c4c(c(Cl)cc23)-c2c(F)ccc(CNC)c2CO4)[C@@H](C)C1] 70 [M+H]⁺: 631.3 | 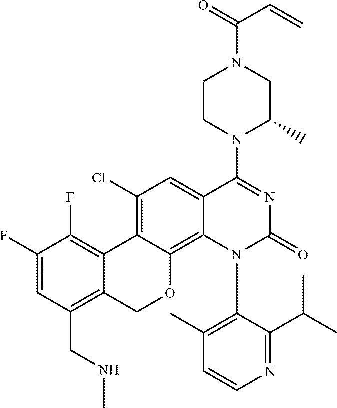 [SMILES: C=CC(=O)N1CCN(c2nc(=O)n(-c3c(C)ccnc3C(C)C)c3c4c(c(Cl)cc23)-c2c(F)c(F)cc(CNC)c2CO4)[C@@H](C)C1] 71 [M+H]⁺: 649.3 | 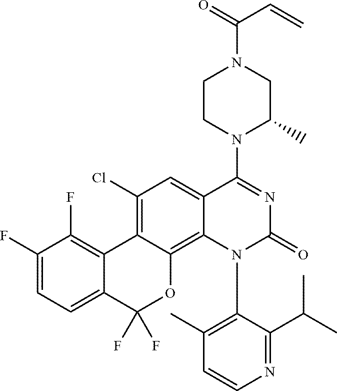 [SMILES: C=CC(=O)N1CCN(c2nc(=O)n(-c3c(C)ccnc3C(C)C)c3c4c(c(Cl)cc23)-c2c(ccc(F)c2F)C(F)(F)O4)[C@@H](C)C1] 72 [M+H]⁺: 642.2 |
| 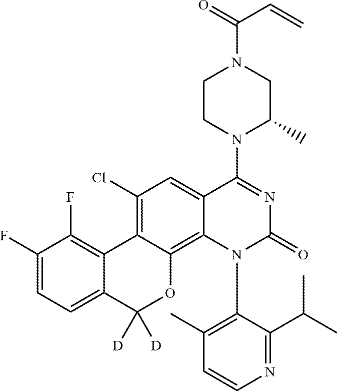 [SMILES: [2H]C1([2H])Oc2c(c(Cl)cc3c(N4CCN(C(=O)C=C)C[C@@H]4C)nc(=O)n(-c4c(C)ccnc4C(C)C)c23)-c2c1ccc(F)c2F] 73 [M+H]⁺: 608.2 | 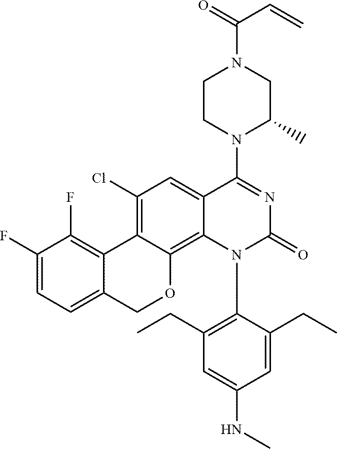 [SMILES: C=CC(=O)N1CCN(c2nc(=O)n(-c3c(CC)cc(NC)cc3CC)c3c4c(c(Cl)cc23)-c2c(ccc(F)c2F)CO4)[C@@H](C)C1] 74 [M+H]⁺: 633.2 | 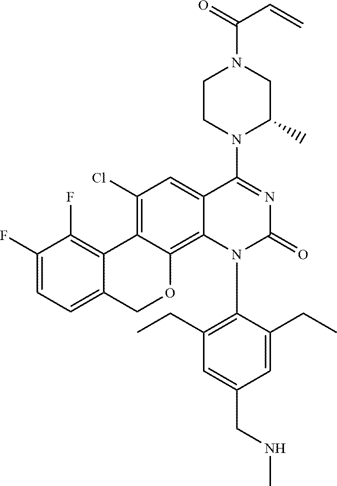 [SMILES: C=CC(=O)N1CCN(c2nc(=O)n(-c3c(CC)cc(CNC)cc3CC)c3c4c(c(Cl)cc23)-c2c(ccc(F)c2F)CO4)[C@@H](C)C1] 75 [M+H]⁺: 648.3 | 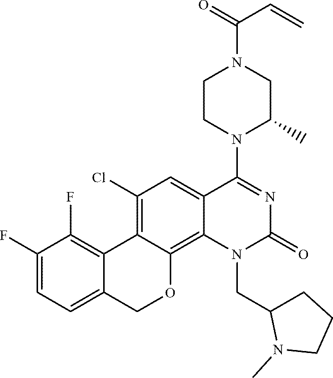 [SMILES: C=CC(=O)N1CCN(c2nc(=O)n(CC3CCCN3C)c3c4c(c(Cl)cc23)-c2c(ccc(F)c2F)CO4)[C@@H](C)C1] 76 [M+H]⁺: 570.2 | 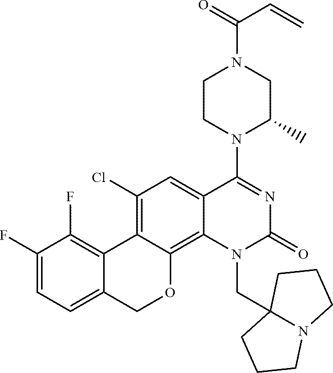 77 [M+H]⁺: 592.2 |
| 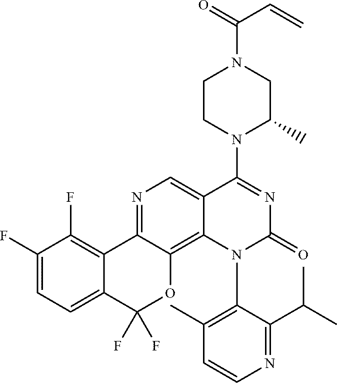 [SMILES: C=CC(=O)N1CCN(c2nc(=O)n(-c3c(C)ccnc3C(C)C)c3c4c(ncc23)-c2c(ccc(F)c2F)C(F)(F)O4)[C@@H](C)C1] 78 [M+H]⁺: 609.2 | 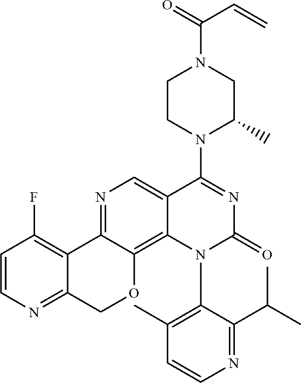 [SMILES: C=CC(=O)N1CCN(c2nc(=O)n(-c3c(C)ccnc3C(C)C)c3c4c(ncc23)-c2c(F)ccnc2CO4)[C@@H](C)C1] 79 [M+H]⁺: 556.2 | 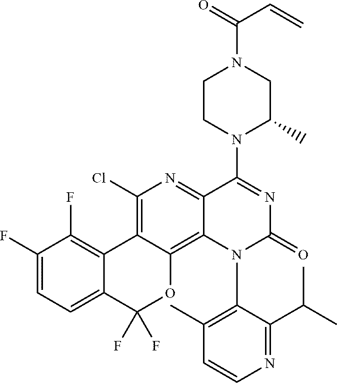 [SMILES: C=CC(=O)N1CCN(c2nc(=O)n(-c3c(C)ccnc3C(C)C)c3c4c(c(Cl)nc23)-c2c(ccc(F)c2F)C(F)(F)O4)[C@@H](C)C1] 80 [M+H]⁺: 643.2 | 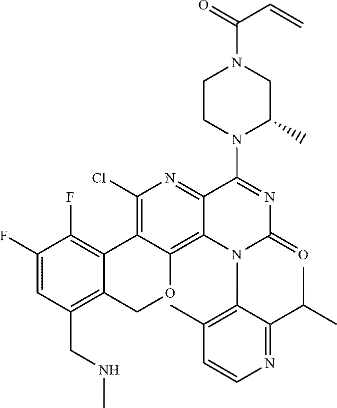 [SMILES: C=CC(=O)N1CCN(c2nc(=O)n(-c3c(C)ccnc3C(C)C)c3c4c(c(Cl)nc23)-c2c(F)c(F)cc(CNC)c2CO4)[C@@H](C)C1] 81 [M+H]⁺: 650.3 | 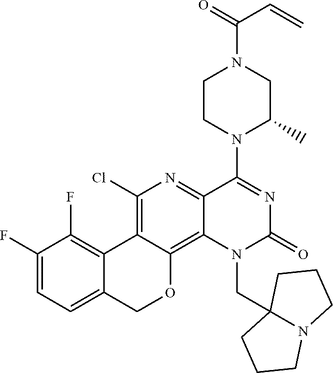 [SMILES: C=CC(=O)N1CCN(c2nc(=O)n(CC34CCCN3CCC4)c3c4c(c(Cl)nc23)-c2c(ccc(F)c2F)CO4)[C@@H](C)C1] 82 [M+H]⁺: 597.2 |
| 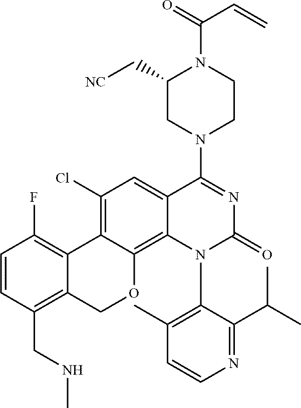 83 [M+H]⁺: 656.4 | 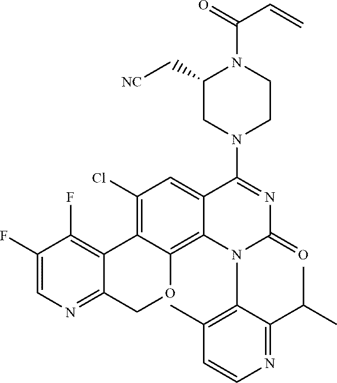 84 [M+H]⁺: 632.2 | 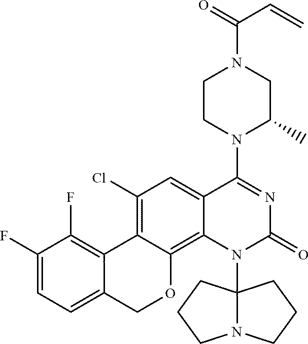 [SMILES: C=CC(=O)N1CCN(c2nc(=O)n(C34CCCN3CCC4)c3c4c(c(Cl)cc23)-c2c(ccc(F)c2F)CO4)[C@@H](C)C1] 85 [M+H]⁺: 582.2 | 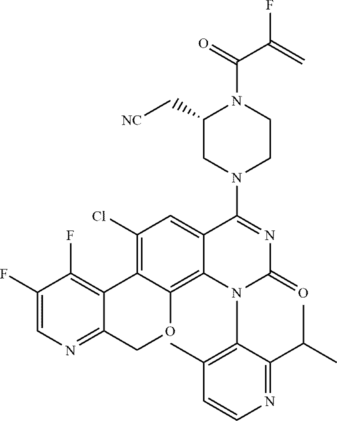 86 [M+H]⁺: 650.2 | 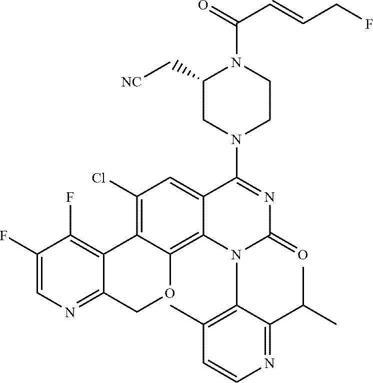 87 [M+H]⁺: 664.2 |
| 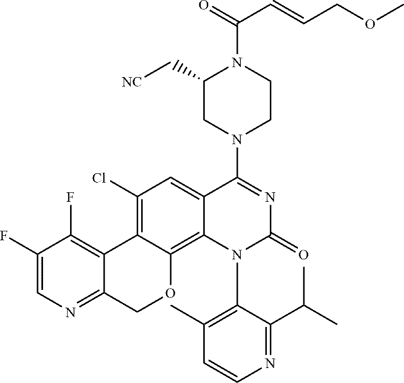 88 [M+H]⁺: 676.2 | 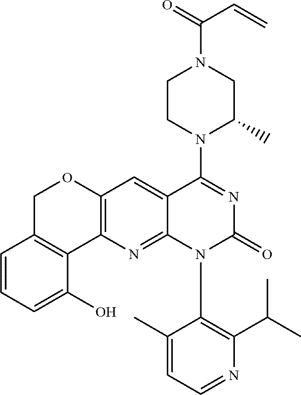 [SMILES: C=CC(=O)N1CCN(c2nc(=O)n(-c3c(C)ccnc3C(C)C)c3nc4c(cc23)OCc2cccc(O)c2-4)[C@@H](C)C1] 89 [M+H]⁺: 553.3 | 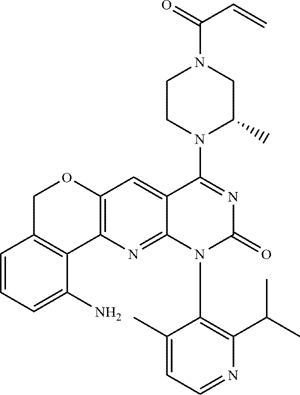 [SMILES: C=CC(=O)N1CCN(c2nc(=O)n(-c3c(C)ccnc3C(C)C)c3nc4c(cc23)OCc2cccc(N)c2-4)[C@@H](C)C1] 90 [M+H]⁺: 552.3 | 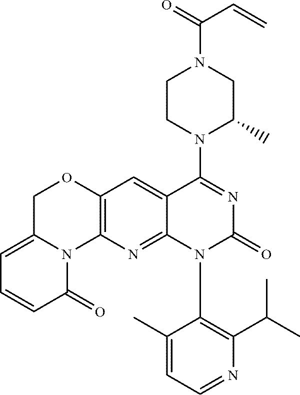 [SMILES: C=CC(=O)N1CCN(c2nc(=O)n(-c3c(C)ccnc3C(C)C)c3nc4c(cc23)OCc2cccc(=O)n2-4)[C@@H](C)C1] 91 [M+H]⁺ : 554.2 | 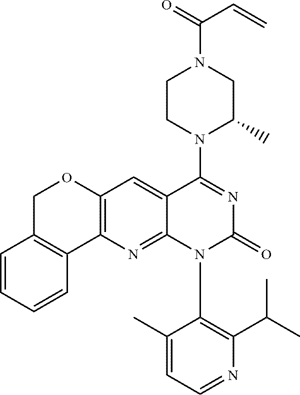 [SMILES: C=CC(=O)N1CCN(c2nc(=O)n(-c3c(C)ccnc3C(C)C)c3nc4c(cc23)OCc2ccccc2-4)[C@@H](C)C1] 92 [M+H]⁺: 537.3 |
| 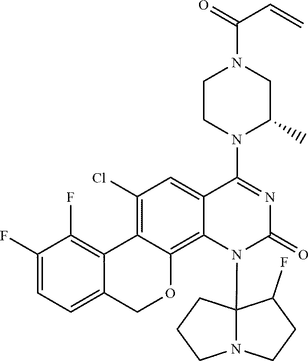 [SMILES: C=CC(=O)N1CCN(c2nc(=O)n(C34CCCN3CCC4F)c3c4c(c(Cl)cc23)-c2c(ccc(F)c2F)CO4)[C@@H](C)C1] 93 [M+H]⁺: 600.2 | 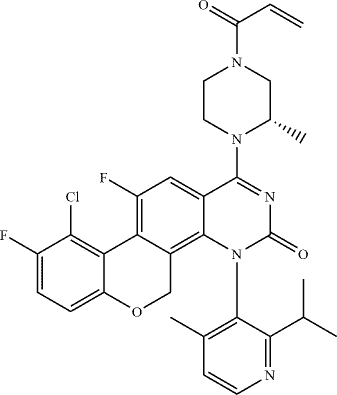 [SMILES: C=CC(=O)N1CCN(c2nc(=O)n(-c3c(C)ccnc3C(C)C)c3c4c(c(F)cc23)-c2c(ccc(F)c2Cl)OC4)[C@@H](C)C1] 94 [M+H]⁺: 606.2 | 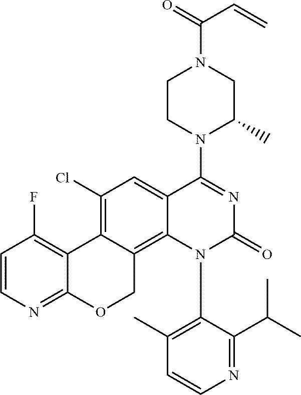 [SMILES: C=CC(=O)N1CCN(c2nc(=O)n(-c3c(C)ccnc3C(C)C)c3c4c(c(Cl)cc23)-c2c(F)ccnc2OC4)[C@@H](C)C1] 95 [M+H]⁺: 589.2 | 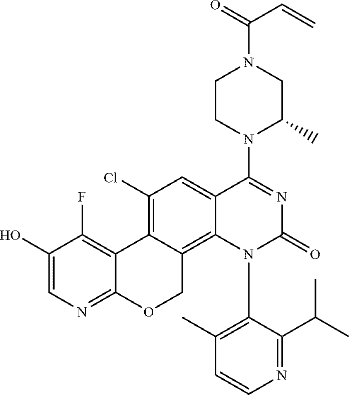 [SMILES: C=CC(=O)N1CCN(c2nc(=O)n(-c3c(C)ccnc3C(C)C)c3c4c(c(Cl)cc23)-c2c(ncc(O)c2F)OC4)[C@@H](C)C1] 96 [M+H]⁺: 605.2 | 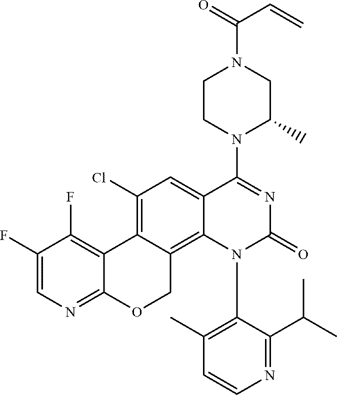 [SMILES: C=CC(=O)N1CCN(c2nc(=O)n(-c3c(C)ccnc3C(C)C)c3c4c(c(Cl)cc23)-c2c(ncc(F)c2F)OC4)[C@@H](C)C1] 97 [M+H]⁺: 607.2 |
| 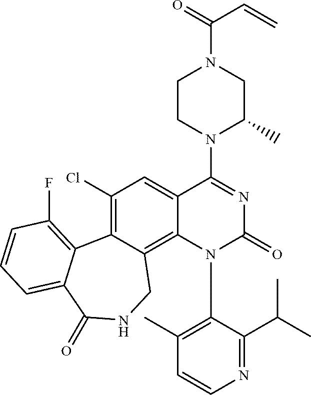 [SMILES: C=CC(=O)N1CCN(c2nc(=O)n(-c3c(C)ccnc3C(C)C)c3c4c(c(Cl)cc23)-c2c(F)cccc2C(=O)NC4)[C@@H](C)C1] 98 [M+H]⁺: 615.2 | 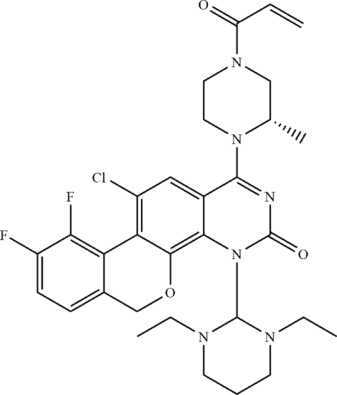 [SMILES: C=CC(=O)N1CCN(c2nc(=O)n(C3N(CC)CCCN3CC)c3c4c(c(Cl)cc23)-c2c(ccc(F)c2F)CO4)[C@@H](C)C1] 99 613.2 | 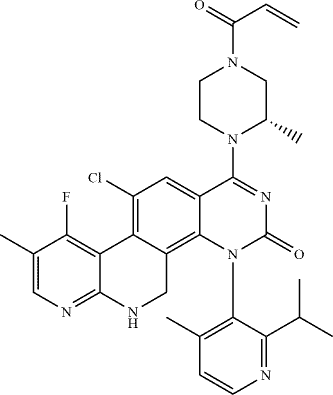 [SMILES: C=CC(=O)N1CCN(c2nc(=O)n(-c3c(C)ccnc3C(C)C)c3c4c(c(Cl)cc23)-c2c(ncc(C)c2F)NC4)[C@@H](C)C1] 100 [M+H]⁺: 602.2 | 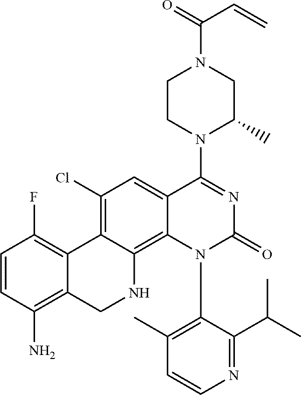 [SMILES: C=CC(=O)N1CCN(c2nc(=O)n(-c3c(C)ccnc3C(C)C)c3c4c(c(Cl)cc23)-c2c(F)ccc(N)c2CN4)[C@@H](C)C1] 101 [M+H]⁺: 602.2 | 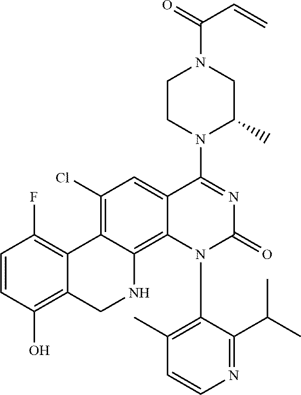 [SMILES: C=CC(=O)N1CCN(c2nc(=O)n(-c3c(C)ccnc3C(C)C)c3c4c(c(Cl)cc23)-c2c(F)ccc(O)c2CN4)[C@@H](C)C1] 102 [M+H]⁺: 603.2 |
| 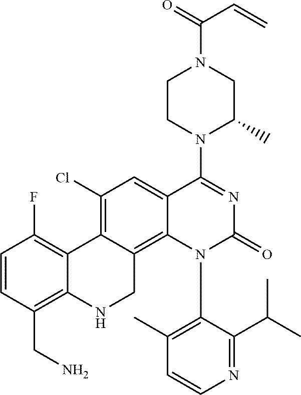 [SMILES: C=CC(=O)N1CCN(c2nc(=O)n(-c3c(C)ccnc3C(C)C)c3c4c(c(Cl)cc23)-c2c(F)ccc(CN)c2NC4)[C@@H](C)C1] 103 [M+H]⁺: 616.3 | 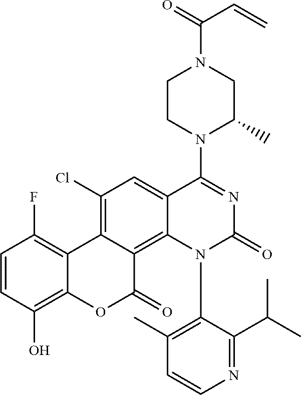 [SMILES: C=CC(=O)N1CCN(c2nc(=O)n(-c3c(C)ccnc3C(C)C)c3c2cc(Cl)c2c4c(F)ccc(O)c4oc(=O)c23)[C@@H](C)C1] 104 [M+H]⁺: 618.2 | 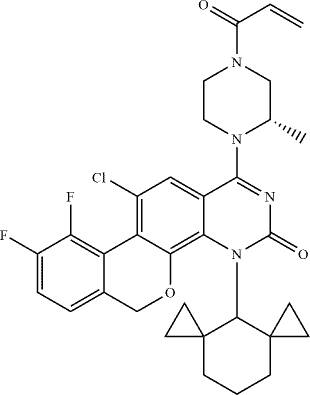 [SMILES: C=CC(=O)N1CCN(c2nc(=O)n(C3C4(CCCC35CC5)CC4)c3c4c(c(Cl)cc23)-c2c(ccc(F)c2F)CO4)[C@@H](C)C1] 105 [M+H]⁺: 607.2 | 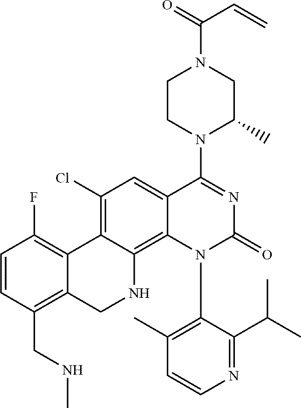 [SMILES: C=CC(=O)N1CCN(c2nc(=O)n(-c3c(C)ccnc3C(C)C)c3c4c(c(Cl)cc23)-c2c(F)ccc(CNC)c2CN4)[C@@H](C)C1] 106 [M+H]⁺: 630.3 | 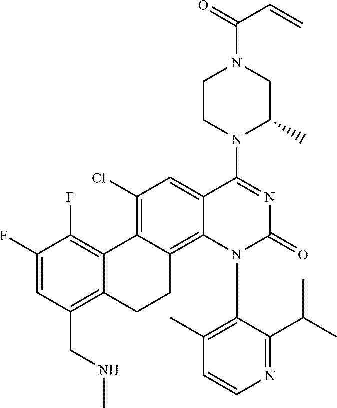 [SMILES: C=CC(=O)N1CCN(c2nc(=O)n(-c3c(C)ccnc3C(C)C)c3c4c(c(Cl)cc23)-c2c(F)c(F)cc(CNC)c2CC4)[C@@H](C)C1] 107 [M+H]⁺: 647.3 |
| 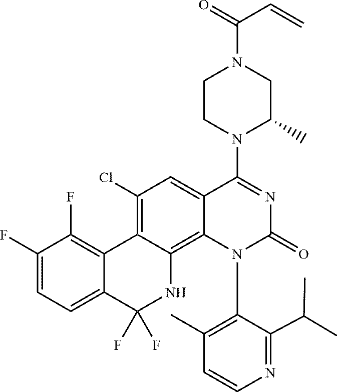 [SMILES: C=CC(=O)N1CCN(c2nc(=O)n(-c3c(C)ccnc3C(C)C)c3c4c(c(Cl)cc23)-c2c(ccc(F)c2F)C(F)(F)N4)[C@@H](C)C1] 108 [M+H]⁺: 641.2 | 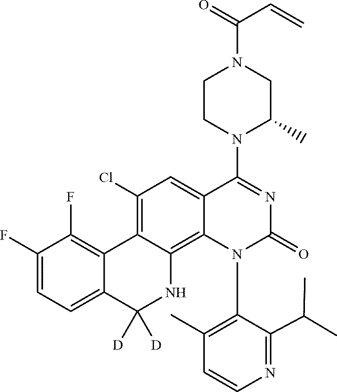 [SMILES: [2H]C1([2H])Nc2c(c(Cl)cc3c(N4CCN(C(=O)C=C)C[C@@H]4C)nc(=O)n(-c4c(C)ccnc4C(C)C)c23)-c2c1ccc(F)c2F] 109 [M+H]⁺: 607.2 | 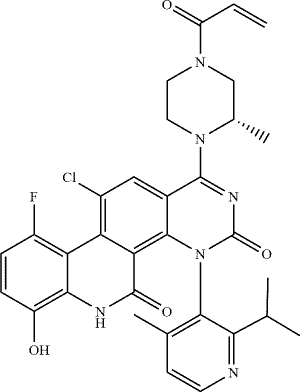 [SMILES: C=CC(=O)N1CCN(c2nc(=O)n(-c3c(C)ccnc3C(C)C)c3c2cc(Cl)c2c4c(F)ccc(O)c4[nH]c(=O)c23)[C@@H](C)C1] 110 [M+H]⁺: 617.2 | 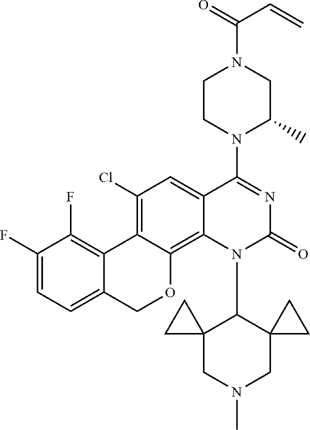 [SMILES: C=CC(=O)N1CCN(c2nc(=O)n(C3C4(CC4)CN(C)CC34CC4)c3c4c(c(Cl)cc23)-c2c(ccc(F)c2F)CO4)[C@@H](C)C1] 111 [M+H]⁺: 622.2 | 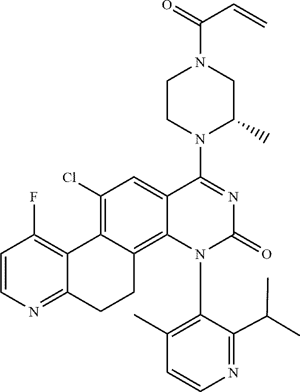 [SMILES: C=CC(=O)N1CCN(c2nc(=O)n(-c3c(C)ccnc3C(C)C)c3c4c(c(Cl)cc23)-c2c(F)ccnc2CC4)[C@@H](C)C1] 112 [M+H]⁺: 587.2 |
| 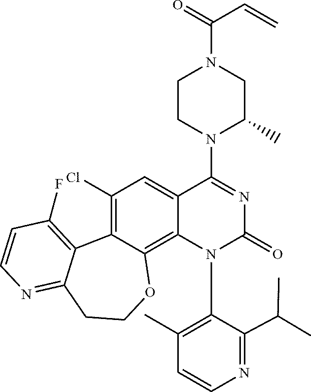 [SMILES: C=CC(=O)N1CCN(c2nc(=O)n(-c3c(C)ccnc3C(C)C)c3c4c(c(Cl)cc23)-c2c(F)ccnc2CCO4)[C@@H](C)C1] 113 [M+H]⁺: 603.2 | 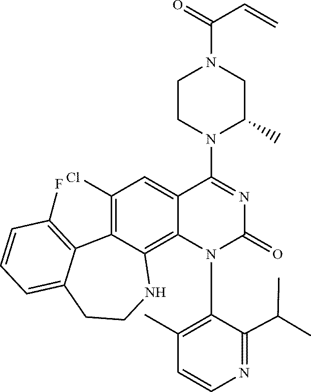 [SMILES: C=CC(=O)N1CCN(c2nc(=O)n(-c3c(C)ccnc3C(C)C)c3c4c(c(Cl)cc23)-c2c(F)cccc2CCN4)[C@@H](C)C1] 114 [M+H]⁺:601.2 | 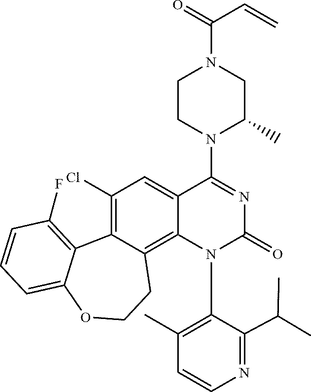 [SMILES: C=CC(=O)N1CCN(c2nc(=O)n(-c3c(C)ccnc3C(C)C)c3c4c(c(Cl)cc23)-c2c(F)cccc2OCC4)[C@@H](C)C1] 115 [M+H]⁺: 602.2 | 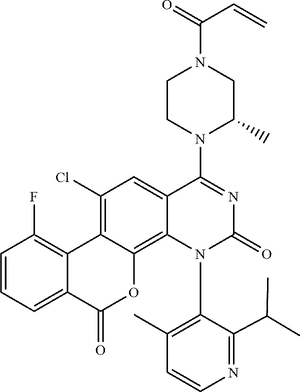 [SMILES: C=CC(=O)N1CCN(c2nc(=O)n(-c3c(C)ccnc3C(C)C)c3c2cc(Cl)c2c3oc(=O)c3cccc(F)c32)[C@@H](C)C1] 116 [M+H]⁺: 602.2 | 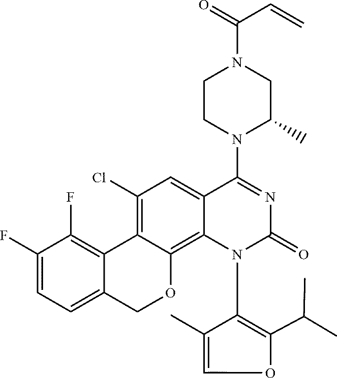 [SMILES: C=CC(=O)N1CCN(c2nc(=O)n(-c3c(C)coc3C(C)C)c3c4c(c(Cl)cc23)-c2c(ccc(F)c2F)CO4)[C@@H](C)C1] 117 [M+H]⁺: 595.2 |
| 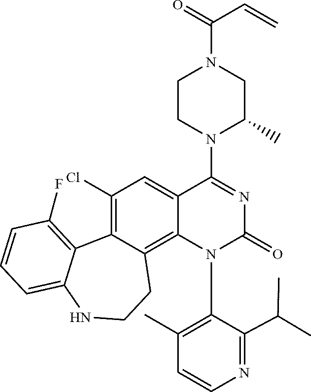 [SMILES: C=CC(=O)N1CCN(c2nc(=O)n(-c3c(C)ccnc3C(C)C)c3c4c(c(Cl)cc23)-c2c(F)cccc2NCC4)[C@@H](C)C1] 118 [M+H]⁺: 601.2 | 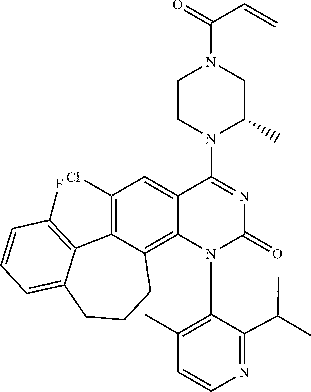 [SMILES: C=CC(=O)N1CCN(c2nc(=O)n(-c3c(C)ccnc3C(C)C)c3c4c(c(Cl)cc23)-c2c(F)cccc2CCC4)[C@@H](C)C1] 119 [M+H]⁺: 600.3 | 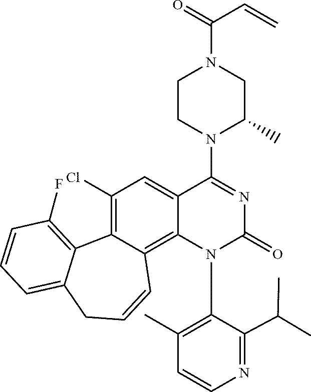 [SMILES: C=CC(=O)N1CCN(c2nc(=O)n(-c3c(C)ccnc3C(C)C)c3c4c(c(Cl)cc23)-c2c(F)cccc2CC=C4)[C@@H](C)C1] 120 [M+H]⁺: 598.2 | 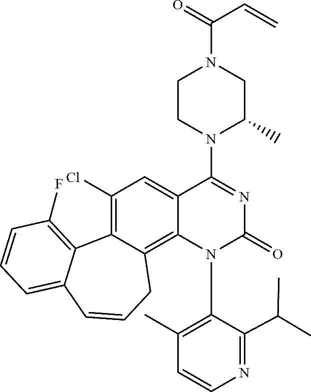 [SMILES: C=CC(=O)N1CCN(c2nc(=O)n(-c3c(C)ccnc3C(C)C)c3c4c(c(Cl)cc23)-c2c(F)cccc2C=CC4)[C@@H](C)C1] 121 [M+H]⁺: 598.2 | 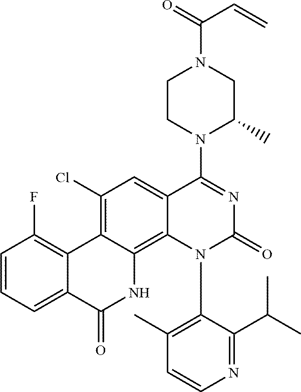 [SMILES: C=CC(=O)N1CCN(c2nc(=O)n(-c3c(C)ccnc3C(C)C)c3c2cc(Cl)c2c3[nH]c(=O)c3cccc(F)c32)[C@@H](C)C1] 122 [M+H]⁺: 601.2 |
| 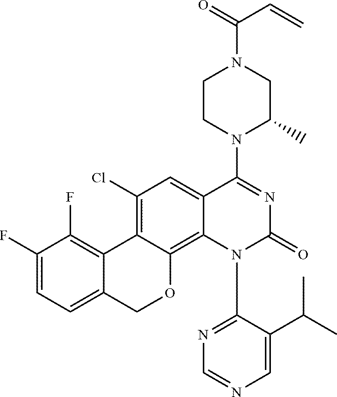 [SMILES: C=CC(=O)N1CCN(c2nc(=O)n(-c3ncncc3C(C)C)c3c4c(c(Cl)cc23)-c2c(ccc(F)c2F)CO4)[C@@H](C)C1] 123 [M+H]⁺: 593.2 | 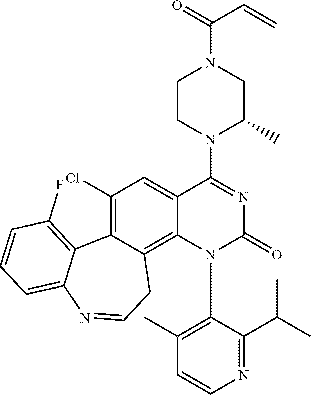 [SMILES: C=CC(=O)N1CCN(c2nc(=O)n(-c3c(C)ccnc3C(C)C)c3c4c(c(Cl)cc23)-c2c(F)cccc2N=CC4)[C@@H](C)C1] 124 [M+H]⁺: 599.2 | 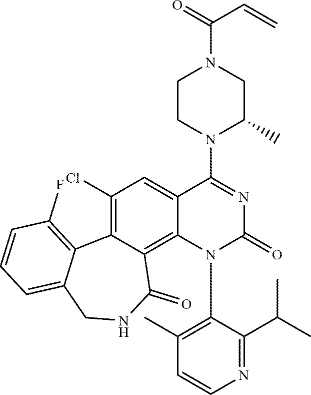 [SMILES: C=CC(=O)N1CCN(c2nc(=O)n(-c3c(C)ccnc3C(C)C)c3c4c(c(Cl)cc23)-c2c(F)cccc2CNC4=O)[C@@H](C)C1] 125 [M+H]⁺: 615.2 | 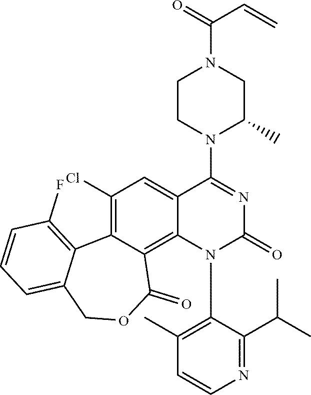 [SMILES: C=CC(=O)N1CCN(c2nc(=O)n(-c3c(C)ccnc3C(C)C)c3c4c(c(Cl)cc23)-c2c(F)cccc2COC4=O)[C@@H](C)C1] 126 [M+H]⁺: 616.2 | 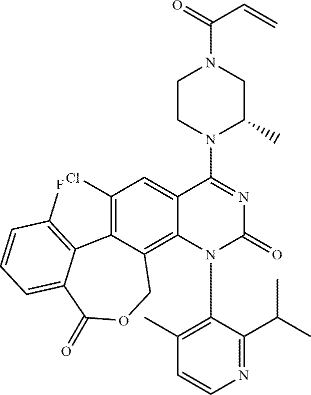 [SMILES: C=CC(=O)N1CCN(c2nc(=O)n(-c3c(C)ccnc3C(C)C)c3c4c(c(Cl)cc23)-c2c(F)cccc2C(=O)OC4)[C@@H](C)C1] 127 [M+H]⁺: 616.2 |
| 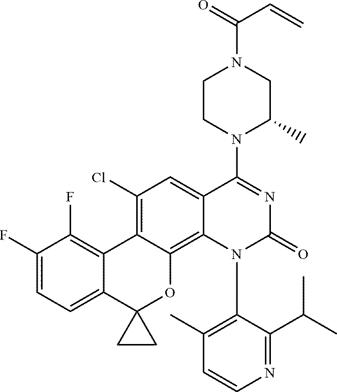 [SMILES: C=CC(=O)N1CCN(c2nc(=O)n(-c3c(C)ccnc3C(C)C)c3c4c(c(Cl)cc23)-c2c(ccc(F)c2F)C2(CC2)O4)[C@@H](C)C1] 128 [M+H]⁺: 632.2 | 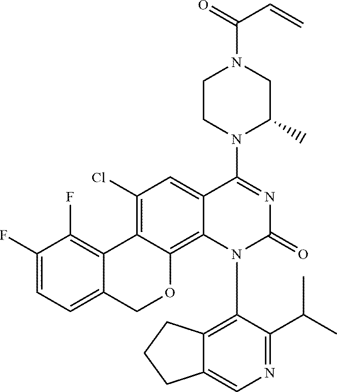 [SMILES: C=CC(=O)N1CCN(c2nc(=O)n(-c3c(C(C)C)ncc4c3CCC4)c3c4c(c(Cl)cc23)-c2c(ccc(F)c2F)CO4)[C@@H](C)C1] 129 [M+H]⁺: 632.2 | 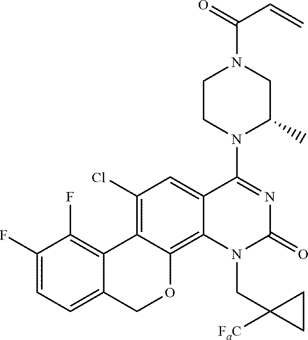 130 [M+H]⁺: 595.2 | 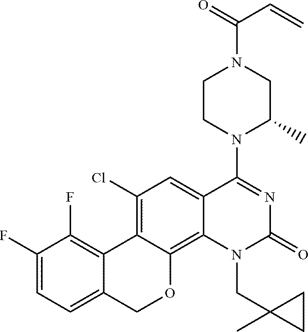 [SMILES: C=CC(=O)N1CCN(c2nc(=O)n(CC3(C)CC3)c3c4c(c(Cl)cc23)-c2c(ccc(F)c2F)CO4)[C@@H](C)C1] 131 [M+H]⁺: 541.2 | 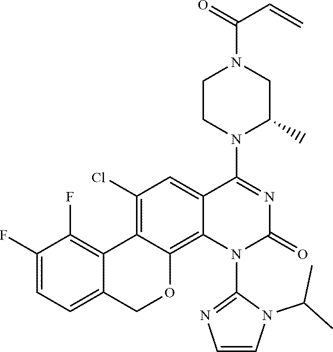 [SMILES: C=CC(=O)N1CCN(c2nc(=O)n(-c3nccn3C(C)C)c3c4c(c(Cl)cc23)-c2c(ccc(F)c2F)CO4)[C@@H](C)C1] 134 [M+H]⁺: 581.2 |
| 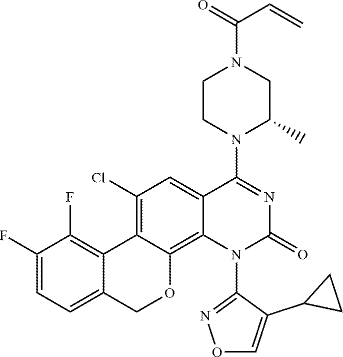 [SMILES: C=CC(=O)N1CCN(c2nc(=O)n(-c3nocc3C3CC3)c3c4c(c(Cl)cc23)-c2c(ccc(F)c2F)CO4)[C@@H](C)C1] 135 [M+H]⁺: 580.2 | 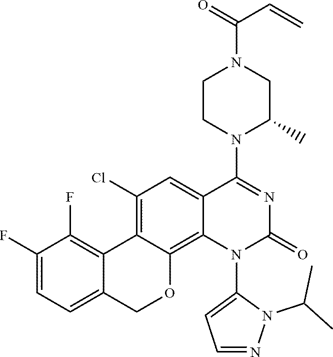 [SMILES: C=CC(=O)N1CCN(c2nc(=O)n(-c3ccnn3C(C)C)c3c4c(c(Cl)cc23)-c2c(ccc(F)c2F)CO4)[C@@H](C)C1] 136 [M+H]⁺: 581.2 | 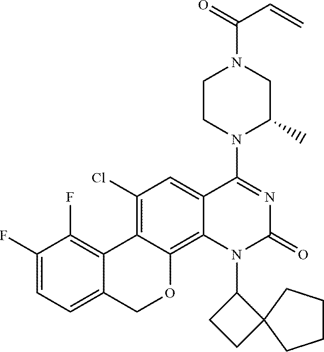 [SMILES: C=CC(=O)N1CCN(c2nc(=O)n(C3CCC34CCCC4)c3c4c(c(Cl)cc23)-c2c(ccc(F)c2F)CO4)[C@@H](C)C1] 137 [M+H]⁺: 581.2 | 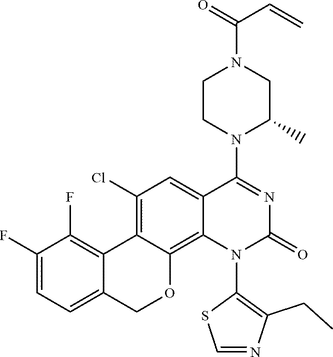 [SMILES: C=CC(=O)N1CCN(c2nc(=O)n(-c3scnc3CC)c3c4c(c(Cl)cc23)-c2c(ccc(F)c2F)CO4)[C@@H](C)C1] 138 [M+H]⁺: 584.2 | 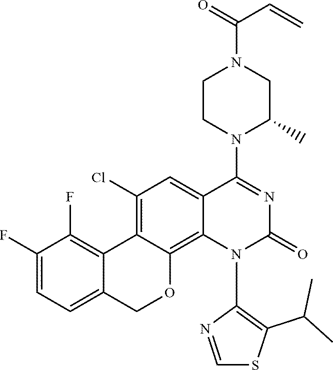 [SMILES: C=CC(=O)N1CCN(c2nc(=O)n(-c3ncsc3C(C)C)c3c4c(c(Cl)cc23)-c2c(ccc(F)c2F)CO4)[C@@H](C)C1] 139 [M+H]⁺: 598.1 |
| 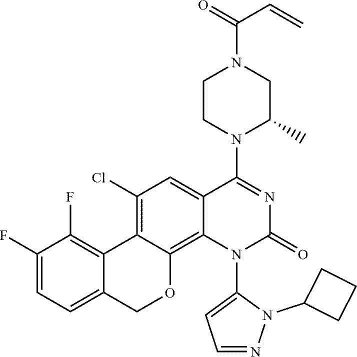 [SMILES: C=CC(=O)N1CCN(c2nc(=O)n(-c3ccnn3C3CCC3)c3c4c(c(Cl)cc23)-c2c(ccc(F)c2F)CO4)[C@@H](C)C1] 140 [M+H]⁺: 593.2 | 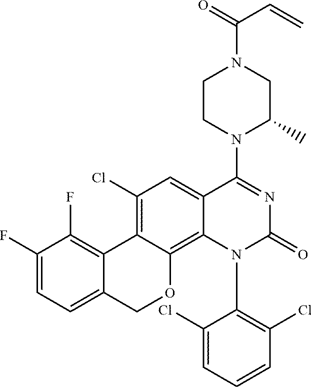 [SMILES: C=CC(=O)N1CCN(c2nc(=O)n(-c3c(Cl)cccc3Cl)c3c4c(c(Cl)cc23)-c2c(ccc(F)c2F)CO4)[C@@H](C)C1] 141 [M+H]⁺: 617.1 | 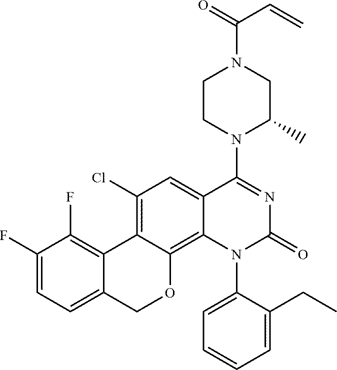 [SMILES: C=CC(=O)N1CCN(c2nc(=O)n(-c3ccccc3CC)c3c4c(c(Cl)cc23)-c2c(ccc(F)c2F)CO4)[C@@H](C)C1] 142 [M+H]⁺: 577.2 | 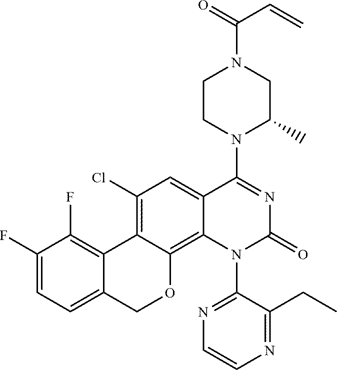 [SMILES: C=CC(=O)N1CCN(c2nc(=O)n(-c3nccnc3CC)c3c4c(c(Cl)cc23)-c2c(ccc(F)c2F)CO4)[C@@H](C)C1] 143 [M+H] : 579.2 | 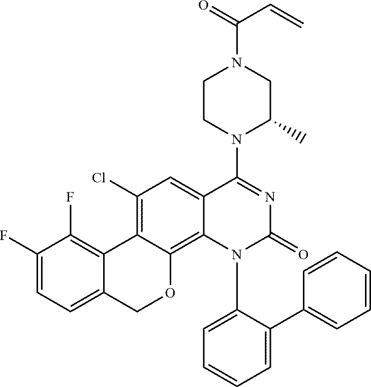 [SMILES: C=CC(=O)N1CCN(c2nc(=O)n(-c3ccccc3-c3ccccc3)c3c4c(c(Cl)cc23)-c2c(ccc(F)c2F)CO4)[C@@H](C)C1] 144 [M+H] : 625.2 |
| 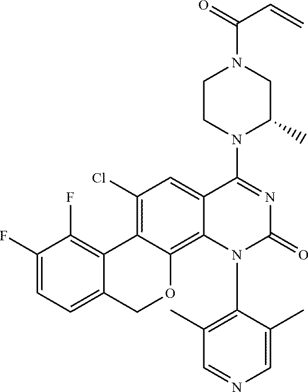 [SMILES: C=CC(=O)N1CCN(c2nc(=O)n(-c3c(C)cncc3C)c3c4c(c(Cl)cc23)-c2c(ccc(F)c2F)CO4)[C@@H](C)C1] 145 [M+H]⁺: 578.2 | 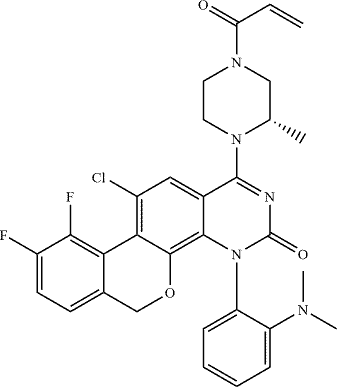 [SMILES: C=CC(=O)N1CCN(c2nc(=O)n(-c3ccccc3N(C)C)c3c4c(c(Cl)cc23)-c2c(ccc(F)c2F)CO4)[C@@H](C)C1] 146 [M+H]⁺: 592.2 | 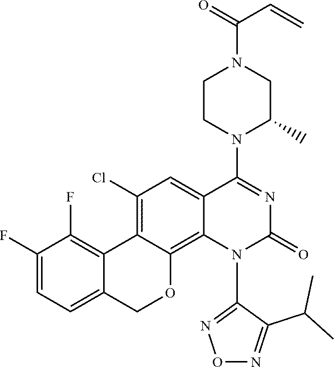 [SMILES: C=CC(=O)N1CCN(c2nc(=O)n(-c3nonc3C(C)C)c3c4c(c(Cl)cc23)-c2c(ccc(F)c2F)CO4)[C@@H](C)C1] 147 [M+H]⁺: 583.2 | 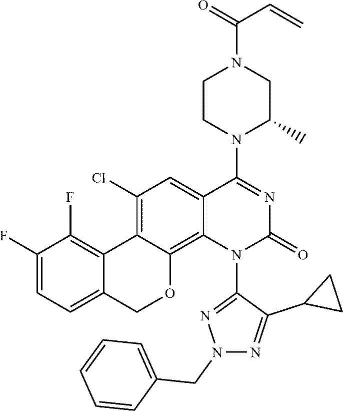 [SMILES: C=CC(=O)N1CCN(c2nc(=O)n(-c3nn(Cc4ccccc4)nc3C3CC3)c3c4c(c(Cl)cc23)-c2c(ccc(F)c2F)CO4)[C@@H](C)C1] 148 [M+H]⁺: 669.2 | 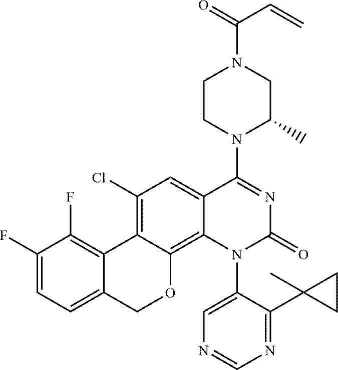 [SMILES: C=CC(=O)N1CCN(c2nc(=O)n(-c3cncnc3C3(C)CC3)c3c4c(c(Cl)cc23)-c2c(ccc(F)c2F)CO4)[C@@H](C)C1] 149 [M+H]⁺: 605.2 |
| 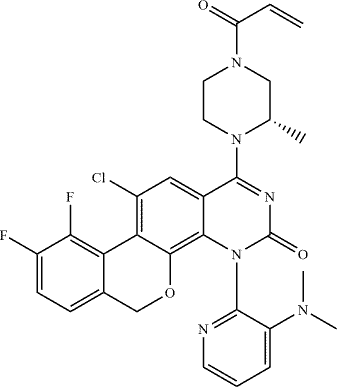 [SMILES: C=CC(=O)N1CCN(c2nc(=O)n(-c3ncccc3N(C)C)c3c4c(c(Cl)cc23)-c2c(ccc(F)c2F)CO4)[C@@H](C)C1] 150 [M+H]⁺: 592.2 | 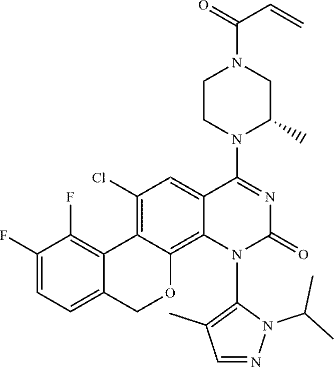 [SMILES: C=CC(=O)N1CCN(c2nc(=O)n(-c3c(C)cnn3C(C)C)c3c4c(c(Cl)cc23)-c2c(ccc(F)c2F)CO4)[C@@H](C)C1] 151 [M+H]⁺:595.2 | 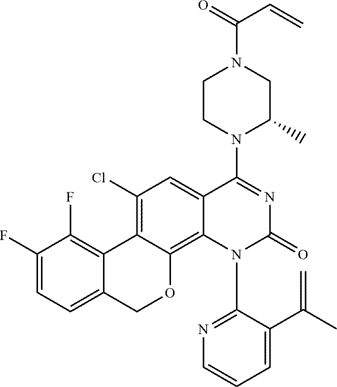 [SMILES: C=CC(=O)N1CCN(c2nc(=O)n(-c3ncccc3C(=C)C)c3c4c(c(Cl)cc23)-c2c(ccc(F)c2F)CO4)[C@@H](C)C1] 152 [M+H]⁺: 590.2 | 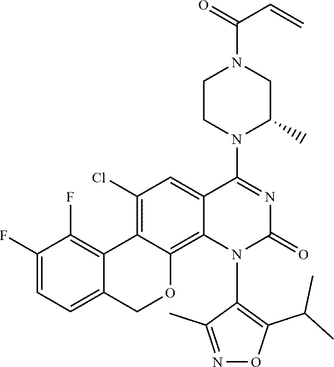 [SMILES: C=CC(=O)N1CCN(c2nc(=O)n(-c3c(C)noc3C(C)C)c3c4c(c(Cl)cc23)-c2c(ccc(F)c2F)CO4)[C@@H](C)C1] 153 [M+H]⁺: 596.2 | 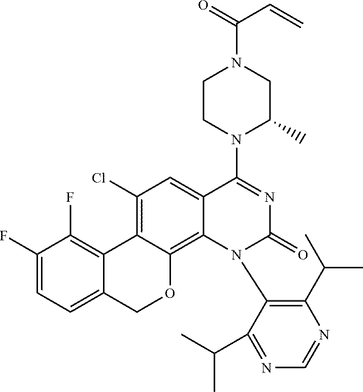 [SMILES: C=CC(=O)N1CCN(c2nc(=O)n(-c3c(C(C)C)ncnc3C(C)C)c3c4c(c(Cl)cc23)-c2c(ccc(F)c2F)CO4)[C@@H](C)C1] 154 [M+H]⁺: 635.2 |
| 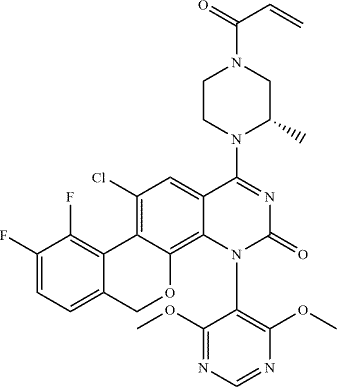 [SMILES: C=CC(=O)N1CCN(c2nc(=O)n(-c3c(OC)ncnc3OC)c3c4c(c(Cl)cc23)-c2c(ccc(F)c2F)CO4)[C@@H](C)C1] 155 [M+H]⁺: 611.2 | 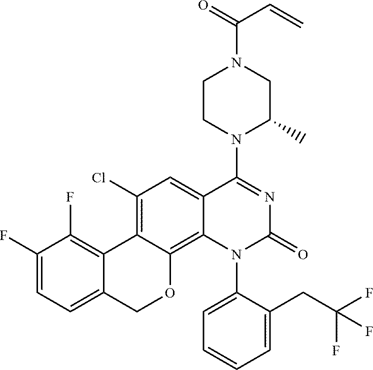 [SMILES: C=CC(=O)N1CCN(c2nc(=O)n(-c3ccccc3CC(F)(F)F)c3c4c(c(Cl)cc23)-c2c(ccc(F)c2F)CO4)[C@@H](C)C1] 156 [M+H]⁺: 631.2 | 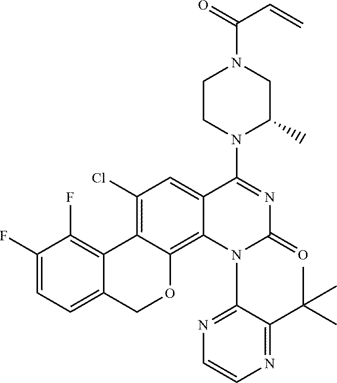 [SMILES: C=CC(=O)N1CCN(c2nc(=O)n(-c3nccnc3C(C)(C)C)c3c4c(c(Cl)cc23)-c2c(ccc(F)c2F)CO4)[C@@H](C)C1] 157 [M+H]⁺: 607.2 | 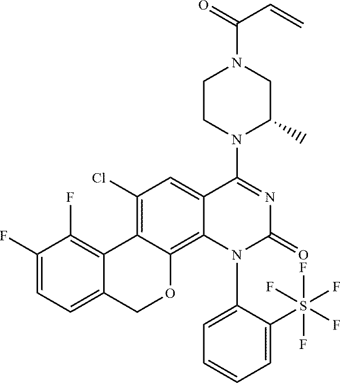 [SMILES: C=CC(=O)N1CCN(c2nc(=O)n(-c3ccccc3S(F)(F)(F)(F)F)c3c4c(c(Cl)cc23)-c2c(ccc(F)c2F)CO4)[C@@H](C)C1] 158 [M+H]⁺:675.2 | 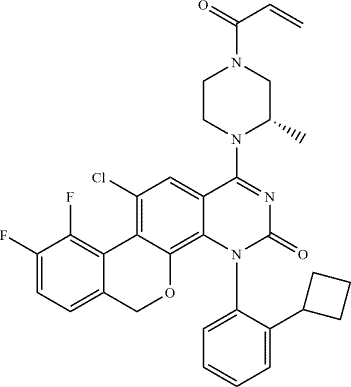 [SMILES: C=CC(=O)N1CCN(c2nc(=O)n(-c3ccccc3C3CCC3)c3c4c(c(Cl)cc23)-c2c(ccc(F)c2F)CO4)[C@@H](C)C1] 159 [M+H]⁺: 603.2 |
| 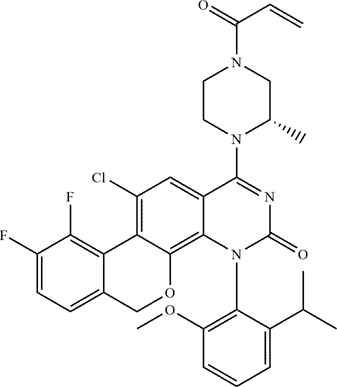 [SMILES: C=CC(=O)N1CCN(c2nc(=O)n(-c3c(OC)cccc3C(C)C)c3c4c(c(Cl)cc23)-c2c(ccc(F)c2F)CO4)[C@@H](C)C1] 160 [M+H]⁺: 621.2 | 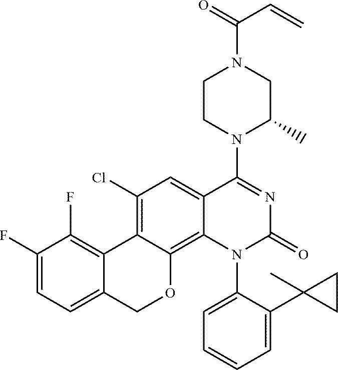 [SMILES: C=CC(=O)N1CCN(c2nc(=O)n(-c3ccccc3C3(C)CC3)c3c4c(c(Cl)cc23)-c2c(ccc(F)c2F)CO4)[C@@H](C)C1] 161 [M+H]⁺: 603.2 | 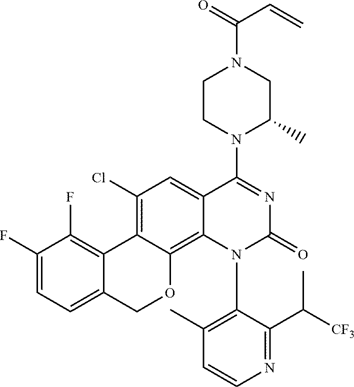 [SMILES: C=CC(=O)N1CCN(c2nc(=O)n(-c3c(C)ccnc3C(C)C(F)(F)F)c3c4c(c(Cl)cc23)-c2c(ccc(F)c2F)CO4)[C@@H](C)C1] 162 [M+H]⁺: 660.2 | 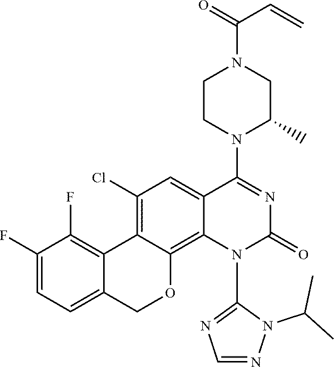 [SMILES: C=CC(=O)N1CCN(c2nc(=O)n(-c3ncnn3C(C)C)c3c4c(c(Cl)cc23)-c2c(ccc(F)c2F)CO4)[C@@H](C)C1] 163 [M+H]⁺: 582.2 | 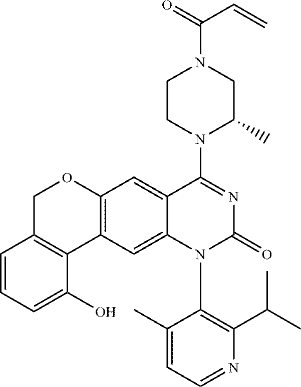 [SMILES: C=CC(=O)N1CCN(c2nc(=O)n(-c3c(C)ccnc3C(C)C)c3cc4c(cc23)OCc2cccc(O)c2-4)[C@@H](C)C1] 164 [M+H]⁺: 552.3 |

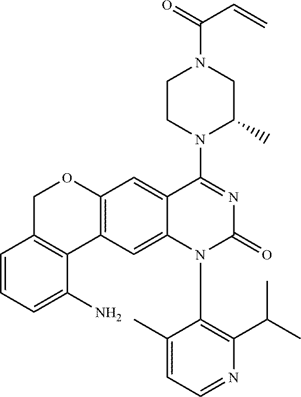 [SMILES: C=CC(=O)N1CCN(c2nc(=O)n(-c3c(C)ccnc3C(C)C)c3cc4c(cc23)OCc2cccc(N)c2-4)[C@@H](C)C1]

165 [M+H]⁺: 551.3

### Biological Assay

### Pharmacological Experiments

### Example 1: Detection of Cell Proliferation Inhibition

MIA PaCa-2 cells were plated in a 96-well ultra-low adsorption plate at 600 cells and 160 µL/well. After overnight incubation, compound solutions with gradient concentrations were prepared, and 40 µL of DMSO solutions of the compounds to be tested were added to each well of cells respectively. Compound final concentrations were 10000, 2000, 400, 80, 16, 3.2, 0.64, 0.12, 0.025, 0 nM (all final concentrations of DMSO were 0.25%). Incubate at 37° C., 5% CO₂ for 96 h. Add 50 µL of Cell-titer Glo working solution to each well, shake and mix well, incubate at room temperature for 10 min, read the Luminescence luminescence value with a multi-function microplate reader, and convert the luminescence value reading into inhibition percentage: Percent Inhibition = (Max-Reading)/(Max-Min)* 100.
“Max” is the DMSO control;
“Min” is the cell-free control.

Curve fitting was performed with Graphpad Prism software and IC₅₀ values were obtained. See Table 1for IC₅₀ data for the inhibition of MIA PaCa-2 cells by the compounds of the examples.

### Example 2: Detection of Cell Proliferation Inhibition

H358 cells were plated in 96-well ultra-low adsorption plates at 2000 cells and 190 µL/well. After overnight incubation, compound solutions of gradient concentrations were prepared, and 10 µL of the DMSO solution of each concentration of the compound to be tested was added to the cells in each well. Compound final concentrations were 10000, 3333.3, 1111.1, 370.4, 123.5, 41.2, 13.7, 4.6, 1.5, 0 nM (all final concentrations of DMSO were 0.25%). Incubate at 37° C., 5% CO₂ for 96 h. Add 50 µL of Cell-titer Glo working solution to each well, incubate at room temperature for 10 min after shaking and mixing, read the Luminescence luminescence value with a multi-function microplate reader, and convert the luminescence value reading into the inhibition percentage:

Percent Inhibition = (Max-Reading)/(Max-Min)* 100.
“Max” is the DMSO control,
“Min” is the cell-free control.

Curve fitting was performed with Graphpad Prism software and IC₅₀ values were obtained. See Table 1 for IC₅₀ data on H358 cell inhibition by example compounds.

TABLE 1

| Compound number | IC₅₀ (nM) | |
|---|---|---|
| | MIA PaCa-2 | H358 |
| 1 | 90.2 | 491 |
| 2 | 122 | 295 |
| 3 | <50 | 78 |
| 4 | 63 | 191 |
| 5 | <50 | 114.8 |
| 6 | 272 | 496 |
| 7 | 70 | 137 |
| 8 | 85 | 155 |
| 9 | >10 µm | >10 µM |
| 10 | 3108 | 3874 |
| 11 | 964 | 2044 |
| 12 | >2 µM | 3177 |
| 13 | <50 | <50 |
| 14 | <50 | <50 |
| 15 | <50 | <50 |
| 16 | <50 | 80.6 |
| 17 | 317.4 | 273.6 |
| 18 | <50 | <50 |
| 19 | <50 | <50 |
| 20 | 64.2 | 62.6 |
| 21 | 1695 | 690 |
| 22 | 2502 | 3737 |
| 23 | 292.2 | 532 |
| 24 | <50 | <50 |
| 25 | 122 | 339 |
| 26 | 546 | 349 |
| 27 | 66 | 110 |
| 28 | 85 | 95 |
| 29 | <50 | <50 |
| 30 | <50 | <50 |
| 31 | <50 | <50 |
| 32 | <50 | <50 |
| 33 | <50 | <50 |
| 34 | 908 | 780 |
| 35 | <50 | <50 |
| 36 | 442 | 889 |
| 37 | 1343 | 2491 |

Although the present invention has been fully described in terms of its embodiments, it is worth noting that various changes and modifications will be apparent to those skilled in the art.

Such changes and modifications are intended to be included within the scope of the appended claims of the present invention.

1. A compound of the Formula (I), its tautomer, deuterated compound or pharmaceutically acceptable salt:
wherein, 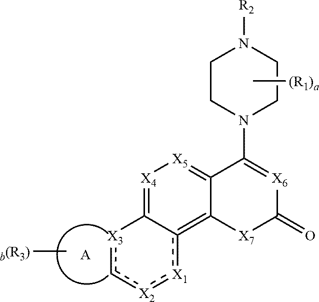
R₁ is independently H, amino, halogen, C₁₋₃ cyano, C₁₋₃ hydroxyalkyl, C₁₋₆ alkyl, C₁₋₆ haloalkyl, C₁₋₆ alkoxy or C₁₋₆ haloalkoxy; or 2 R₁ and the connected atoms together form C₃₋₆ cycloalkyl or 3-6 membered heterocyclyl;
R₂ is acryloyl or substituted acryloyl;
A is selected from the group consisting of C₃₋₁₀ cycloalkyl, 3-10 membered heterocyclyl, C₆₋₁₂ aryl and 5-12 membered heteroaryl;
X₁ is independently—C(R₄)₁₋₂—(CH₂)₀₋₂—, —NR₄—(CH₂)₀₋₂—, —O—(CH₂)₀₋₂—, —S—(CH₂)₀₋₂—, 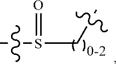 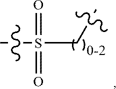 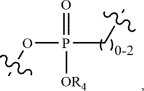 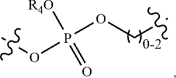 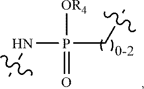 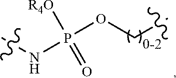 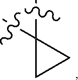
—CH═CH— or —N═CH—;
X₂ is independently —C(R₅)₁₋₂—(CH₂)₀₋₂—, —NR₅—(CH₂)₀₋₂—, —O—(CH₂)₀₋₂—, —S—(CH₂)₀₋₂—, 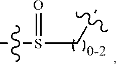 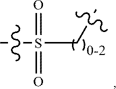 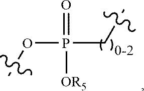 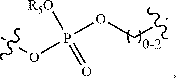 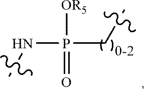 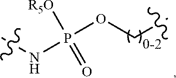 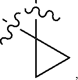
—CH═CH— or —N═CH—;
X₃ is C, CH or N;
X₄ is CR₆ or N;
X₅ is CR₇ or N;
X₆ is CR₈ or N;
X₇ is NR₉ or CHR₉;
R₆ and R₈ are independently selected from the group consisting of H, halogen, C₁₋₃ alkyl, C₁₋₃ haloalkyl, C₁₋₃ alkoxy, C₃₋₄ cycloalkyl, C₃₋₁₂ heterocycloalkyl, C₂₋₃ alkenyl, C₂₋₃ alkynyl, aryl and heteroaryl;
R₇ is independently H, hydroxy, C₁₋₆ alkyl, C₁₋₆ haloalkyl, C₁₋₆ alkoxy, -NH-C₁₋₆ alkyl, -N(C₁₋₆ alkyl)₂, cyano or halogen;
R₉ is independently C₁₋₆ alkyl, —C₀₋₃ alkylene-C₆₋₁₄ aryl, —C₀₋₃ alkylene-(5-14-membered heteroaryl), —C₀₋₃ alkylene-C₃₋₁₄ cycloalkyl, —C₀₋₃ alkylene-(3-14 membered heterocycloalkyl), -O—C₀₋₃ alkylene-C₆₋₁₄ aryl, —O—C₀₋₃ alkylene -(5-14 membered heteroaryl), —O—C₀₋₃ alkylene-C₃₋₁₄ cycloalkyl, —O—C₀₋₃ alkylene-(3-14 membered heterocycloalkyl), —NH—C₁₋₆ alkyl, —N (C₁₋₆ alkyl) ₂, —NH—C₀₋₃ alkylene-C₆₋₁₄ aryl, —NH—C₀₋₃ alkylene-(5-14 membered Heteroaryl), -NH—C₀₋₃ alkylene-C₃₋₁₄ cycloalkyl or —NH—C₀₋₃ alkylene-(3-14 membered heterocycloalkyl), where said R₉ is optionally unsubstituted or further substituted by one or more R₉ₐ substituents;
R₃, R₄, R₅, and R₉ₐ are each independently selected from the group consisting of H, D, oxo, nitro, halogen, C₁₋₆ alkyl, C₁₋₆ cyano, C₁₋₆ haloalkyl, —C₀₋₃ alkylene-ORₐ, —C₀₋₃ alkylene-N(Rₐ)₂, —C₀₋₃ alkylene—NRₐC(═O)Rₐ, —C₀₋₃ alkylene—NRₐC(═O)ORₐ, —C₀₋₃ alkylene—NRₐS(═O)₂Rₐ, —C₀₋₃ alkylene—S(═O)Rₐ, —C₀₋₃ alkylene—S(═O)₂Rₐ, —C₀₋₃ alkylene—S(═O)₂N(Rₐ)₂, —C₀₋₃ alkylene-SRₐ, -C₀₋₃ alkylene-S(Rₐ)₅, —C₀₋₃ alkylene—C(═O)N(Rₐ)₂, —C₀₋₃ alkylene—C(═O)Rₐ, —C₀₋₃ alkylene—C(═O)ORₐ, C₂₋₆ alkenyl, C₂₋₆ alkynyl, —C₀₋₃ alkylene-C₃₋₁₄ cycloalkyl, -C₀₋₃ alkylene-(₃₋₁₄ membered heterocycloalkyl), —C₀₋₃ alkylene-C₆₋₁₄ aryl and —C₀₋₃ alkylene-(5-14 membered heteroaryl), where said —C₀₋₃ alkylene-C₃₋₁₄ cycloalkyl, —C₀₋₃ alkylene-(3-14 membered heterocycloalkyl), —C₀₋₃ alkylene-C₆₋₁₄ aryl or —C₀₋₃ alkylene-(5-14 membered heteroaryl) is optionally unsubstituted or further substituted with one or more Rₐ substituents, and each Rₐ is independently selected from the group consisting of H, halogen, C₁₋₆ alkyl, C₁₋₆ haloalkyl, C₃₋₁₄ cycloalkyl, 3-14 membered heterocycloalkyl, C₂₋₃ alkenyl, C₂₋₃ alkynyl, aryl and heteroaryl;
a and b are each independently selected from the group consisting of 1, 2, 3 and 4; 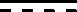
is a double bond or a single bond.

2. The compound as claimed in claim 1, wherein the compound is represented by Formula (I-1), 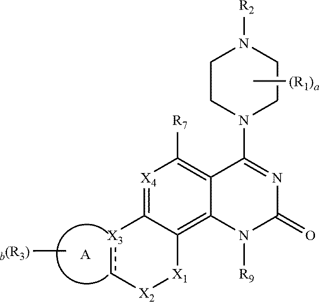
wherein,
R₁ is independently H, amino, halogen, C₁₋₃ cyano, C₁₋₃ hydroxyalkyl or C₁₋₆ alkyl; or 2 R₁ together with the attached atoms form C₃₋₆ cycloalkyl or 3-6 membered heterocyclic group;
R₂ is acryloyl or substituted acryloyl;
ring A is selected from the group consisting of C₃₋₁₀ cycloalkyl, 3-10 membered heterocyclyl, C₆₋₁₂ aryl and 5-12 membered heteroaryl;
X₁ is independently —C(R₄)₂—(CH₂)₀₋₂—, —NR₄—(CH₂)₀₋₂—, —O—(CH₂)₀₋₂—, —S—(CH₂)₀₋₂—, 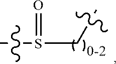 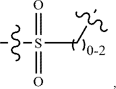 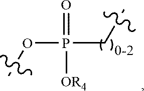 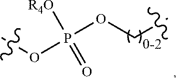 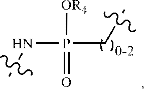 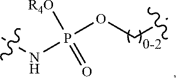 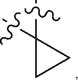
—C(═O)—(CH₂)₀₋₂—, —CH═CH— or —N═CH—, where said R₄ is independently selected from the group consisting of H, D, halogen and C₁₋₃ alkyl;
X₂ is independently —C(R₅)₂—(CH₂)₀₋₂—, —NR₅—(CH₂)₀₋₂—, —O—(CH₂)₀₋₂—, —S—(CH₂)₀₋₂— 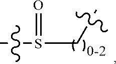 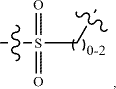 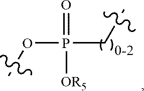 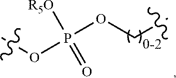 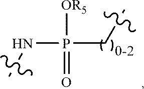 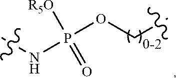 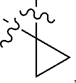
—C(═O)—(CH₂)₀₋₂—, —CH═CH— or —N═CH—, where said R₅ is independently selected from the group consisting of H, D, halogen and C₁₋₃ alkyl;
X₃ is C, CH or N;
X₄ is CR₆ or N;
R₆ is independently H, halogen, C₁₋₃ alkyl, C₁₋₃ haloalkyl, C₁₋₃ alkoxy, C₃₋₄ cycloalkyl, C₃₋₁₂ heterocycloalkyl, C₂₋₃ alkenyl, C₂₋₃ alkynyl, aryl or heteroaryl;
R₇ is independently H, hydroxy, C₁₋₆ alkyl, C₁₋₆ haloalkyl, C₁₋₆ alkoxy, —NH—C₁₋₆ alkyl, —N(C₁₋₆ alkyl)₂, cyano or halogen;
R₉ is independently —C₀₋₃ alkylene-C₆₋₁₄ aryl, —C₀₋₃ alkylene-(5-14-membered heteroaryl), —C₀₋₃ alkylene-C₃₋₁₄ cycloalkyl, —C₀₋₃ alkylene-(3-14 membered heterocycloalkyl), —O—C₀₋₃ alkylene-C₆₋₁₄ aryl, —O—C₀₋₃ alkylene-(5-14 membered heteroaryl), —O—C₀₋₃ alkylene-C₃₋₁₄ cycloalkyl, -O—C₀₋₃ alkylene-(3-14-membered heterocycloalkyl), -NH-C₁₋₆ alkyl, -N(C₁₋₆ alkyl)₂, -NH—C₀₋₃ alkylene-C₆₋₁₄ aryl, -NH—C₀₋₃ alkylene-(5-14-membered heteroaryl), -NH—C₀₋₃ alkylene-C₃₋₁₄ cycloalkyl or -NH—C₀₋₃ alkylene-(3-14-membered heterocycloalkyl), where said R₉ is optionally unsubstituted or further substituted by 1-4 R₉ₐ;
R₃ and R₉ₐ are each independently selected from the group consisting of H, D, oxo, nitro, halogen, C₁₋₆ alkyl, C₁₋₆ cyano, C₁₋₆ haloalkyl, —C₀₋₃ alkylene-ORₐ, —C₀₋₃ alkylene-N(Rₐ)₂, —C₀₋₃ alkylene—NRₐC(═O)Rₐ, —C₀₋₃ alkylene—NRₐC(═O)ORₐ, —C₀₋₃ alkylene—NRₐS(═O)₂Rₐ, —C₀₋₃ alkylene—S(═O)Rₐ, —C₀₋₃ alkylene—S(═O)₂Rₐ, —C₀₋₃ alkylene—S(═O) ₂N(Rₐ)₂, —C₀₋₃ alkylene-SRₐ, -C₀₋₃ alkylene-S(Rₐ)₅, —C₀₋₃ alkylene—C(═O)N(Rₐ)₂, —C₀₋₃ alkylene—C(═O)Rₐ, —C₀₋₃ alkylene—C(═O)ORₐ, C₂₋₆ alkenyl, C₂₋₆ alkynyl, —C₀₋₃ alkylene-C₃₋₁₄ cycloalkyl, —C₀₋₃ alkylene-(3-14 membered heterocycloalkyl), —C₀₋₃ alkylene-C₆₋₁₄ aryl and —C₀₋₃ alkylene-(5-14-membered heteroaryl), where said —C₀₋₃ alkylene-C₃₋₁₄ cycloalkyl, —C₀₋₃ alkylene-(3-14-membered heterocycloalkyl), —C₀₋₃ alkylene -C₆₋₁₄ aryl or —C₀₋₃ alkylene-(5-14 membered heteroaryl) is optionally unsubstituted or further substituted with one or more Rₐ substituents, and each Rₐ is independently H, halogen, C₁₋₆ alkyl, C₁₋₆ haloalkyl, C₃₋₁₄ cycloalkyl, 3-14 membered heterocycloalkyl, C₂₋₃ alkenyl, C₂₋₃ alkynyl, aryl or heteroaryl;
a and b are independently 1, 2, 3 or 4; 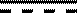
is a double bond or a single bond.

3. The compound as claimed in claim 1, wherein the 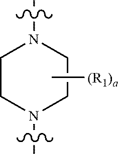
is selected from the group consisting of 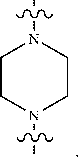 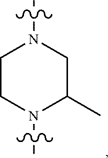 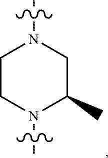 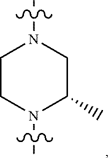 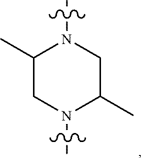 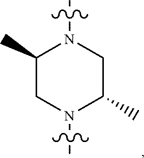 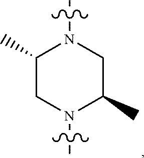 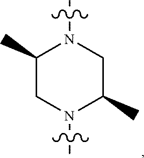 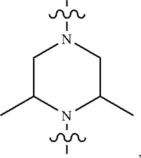 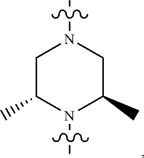 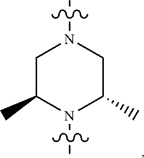 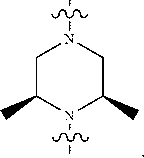 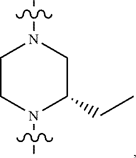 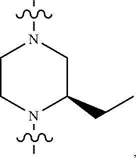 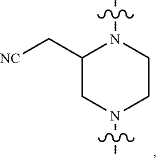 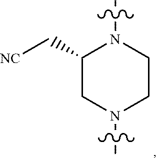 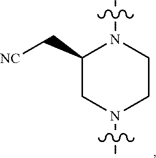 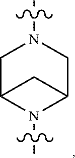 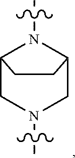
and 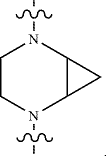
.

4. The compound as claimed in claim 1, wherein
R₂ is selected from the group consisting of 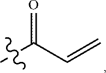 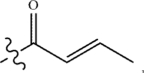 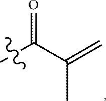 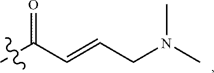 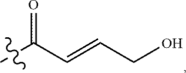 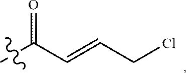 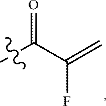 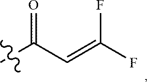 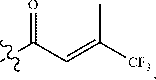 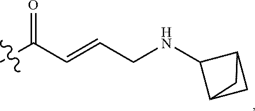 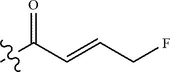
and 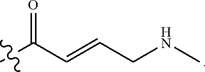
.

5. The compound as claimed in claim 1, wherein
ring A is selected from the group consisting of 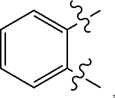 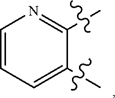 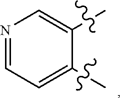 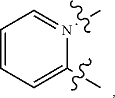 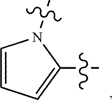 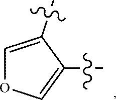 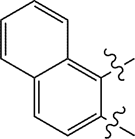
and 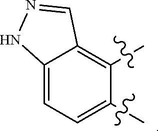
.

6. The compound as claimed in claim 1, wherein
R₃ is selected from the group consisting of H, halogen, oxo, —ORₐ, —C₀₋₃ alkylene-N(Rₐ)₂ and C₁₋₆ haloalkyl, where said Rₐ is independently H, C₁₋₆ alkyl or C₁₋₆ haloalkyl.

7. The compound as claimed in claim 1, wherein
X₁ is selected from the group consisting of —C(R₄)₂—(CH₂)₀₋₂—, —NH—(CH₂)₀₋₂—, —O—(CH₂)₀₋₂—, —C(O)—(CH₂)₀₋₂—, 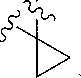
—CH═CH— and —N═CH—, where said R₄ is independently H, D, halogen or C₁₋₃ alkyl.

8. The compound as claimed in claim 1, wherein
X₂ is selected from the group consisting of —C(R₅)₂—(CH₂)₀₋₂—, —NH—(CH₂)₀₋₂—, —O—(CH₂)₀₋₂—, —C(O)—(CH₂)₀₋₂—, 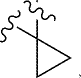
—CH═CH— and —N═CH—, where said R₅ is independently selected from the group consisting of H, D, halogen and C₁₋₃ alkyl.

9. The compound as claimed in claim 1, wherein
X₃ is C, CH or N.

10. The compound as claimed in claim 1, wherein
X₄ is CR₆ or N, and the substituent R₆ is independently halogen, C₁₋₃ alkyl, C₁₋₃ haloalkyl or C₂₋₃ alkenyl.

11. The compound as claimed in claim 1, wherein X₅ is CR₇ or N, and the substituent R₇ is independently H or C₁₋₃ alkyl.

12. The compound as claimed in claim 1, wherein X₆ is CR₈ or N, and the substituent R₈ is independently H, halogen or C₁₋₃ alkyl.

13. The compound as claimed in claim 1, wherein X₇ is NR₉ or CHR₉.

14. The compound as claimed in claim 1, wherein R₉ is selected from the group consisting of —C₀₋₃ alkylene-C₆₋₁₄ aryl, —C₀₋₃ alkylene-(5-14-membered heteroaryl), —C₀₋₃ alkylene-C₃₋₁₄ cycloalkyl, and —C₀₋₃ alkylene-(3-14 membered heterocycloalkyl), where said —C₀₋₃ alkylene-C₆₋₁₄ aryl, —C₀₋₃ alkylene-(5-14 membered heteroaryl), —C₀₋₃ alkylene-C₃₋₁₄ cycloalkyl or —C₀₋₃ alkylene-(3-14 membered heterocycloalkyl) is unsubstituted or further substituted by 1-4 substituents selected from the group consisting of halogen, C₁₋₃ alkyl, C₁₋₃ alkenyl, C₁₋₃ haloalkyl, —(CH₂)₀₋₃—S(Rₐ)₅, C₁₋₃ alkoxy, C₃₋₆ cycloalkyl, C₁₋₃ alkyl substituted C₃₋₆ cycloalkyl, —C₀₋₃ alkylene-phenyl, and —C₀₋₃ alkylene-N(Rₐ)₂, and each Rₐ is independently H or C₁₋₆ alkyl.

15. The compound as claimed in claim 1,
wherein 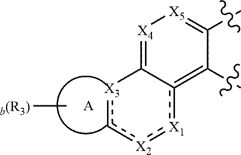
is selected from the group consisting of 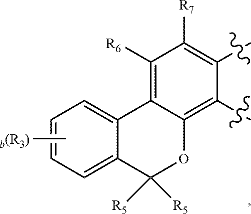 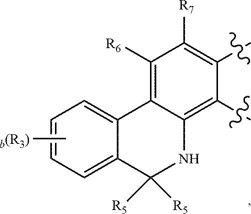 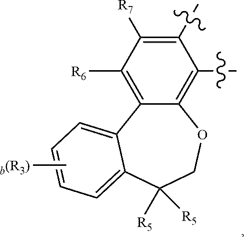 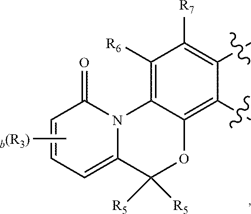 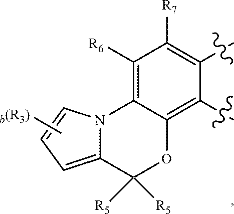 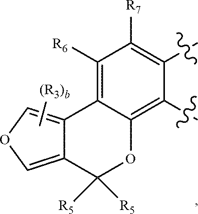 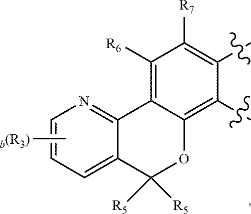 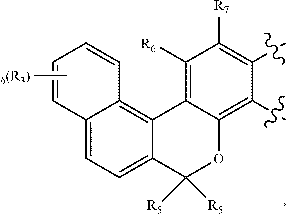 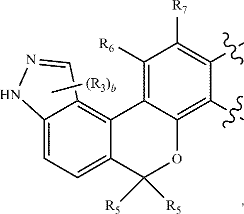 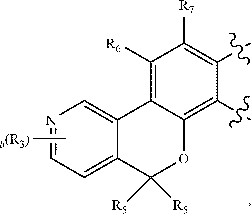 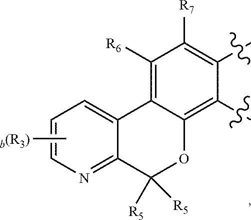 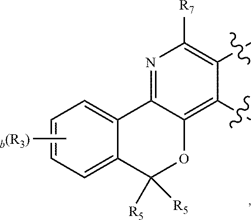 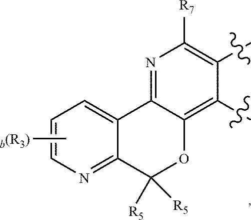 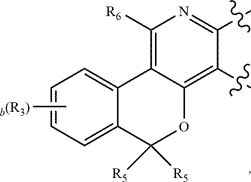 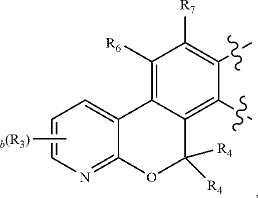 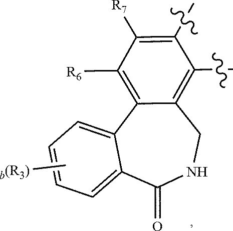 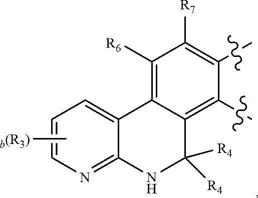 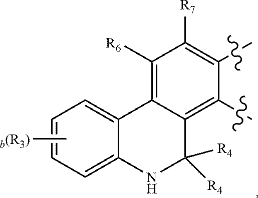 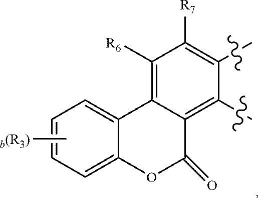 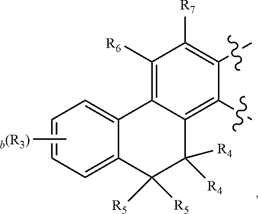 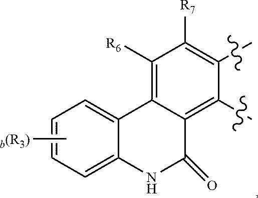 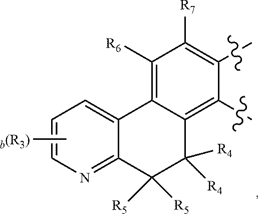 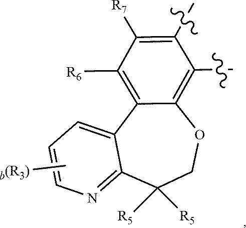 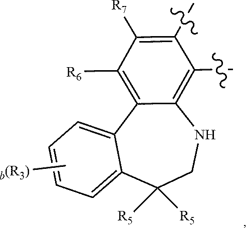 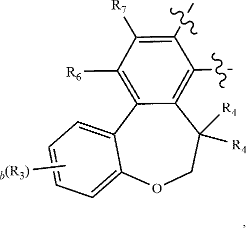 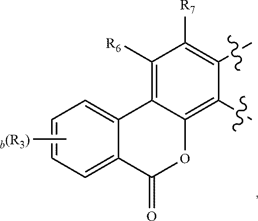 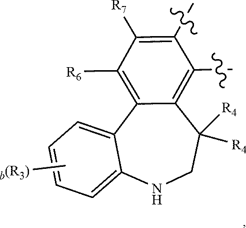 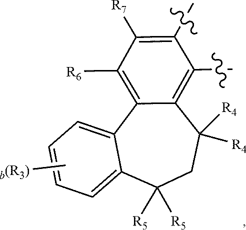 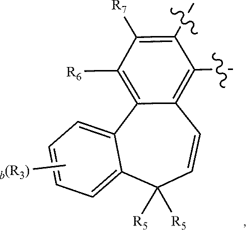 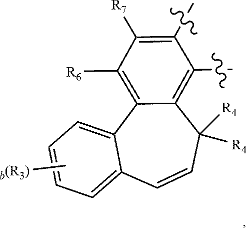 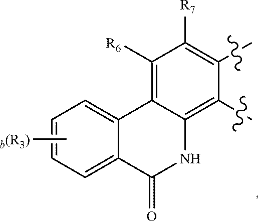 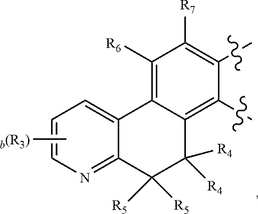 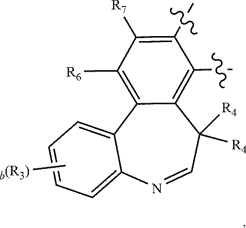 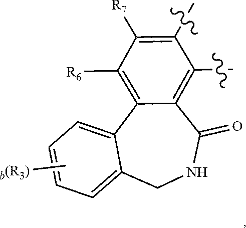 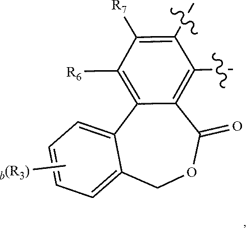 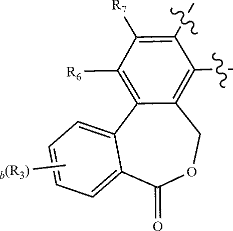
and 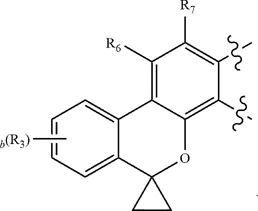
.

16. The compound as claimed in claim 1, wherein the compound is selected from Formula (Ia) to Formula (Ie), 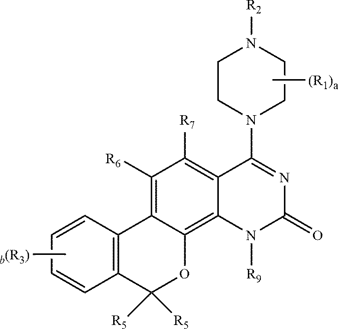 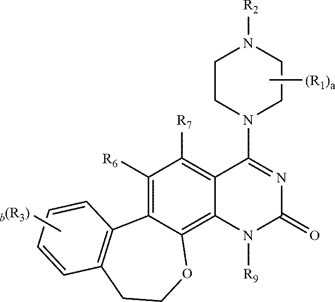 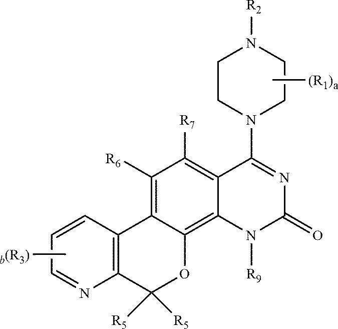 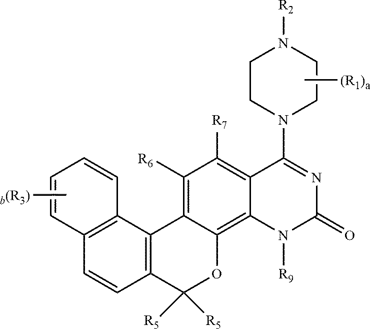 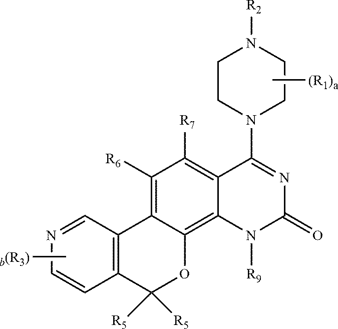
wherein the substituents R₁-R₉, a, and b are as defined in Formula (I).

17. A The compound, its tautomer, deuterated compound or pharmaceutically acceptable salt of claim 1, wherein the compound is selected from the group consisting of: 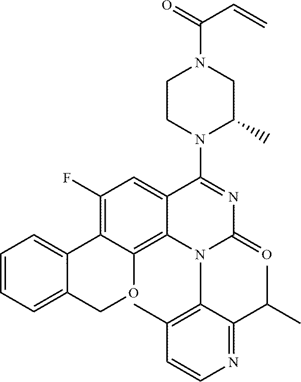 [SMILES: C=CC(=O)N1CCN(c2nc(=O)n(-c3c(C)ccnc3C(C)C)c3c4c(c(F)cc23)-c2ccccc2CO4)[C@@H](C)C1] 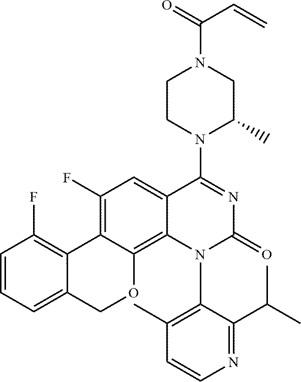 [SMILES: C=CC(=O)N1CCN(c2nc(=O)n(-c3c(C)ccnc3C(C)C)c3c4c(c(F)cc23)-c2c(F)cccc2CO4)[C@@H](C)C1] 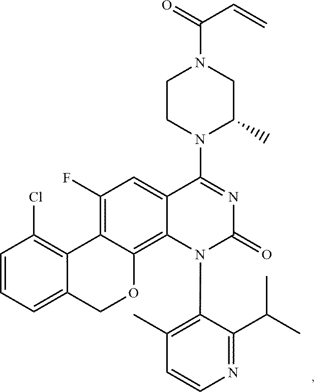 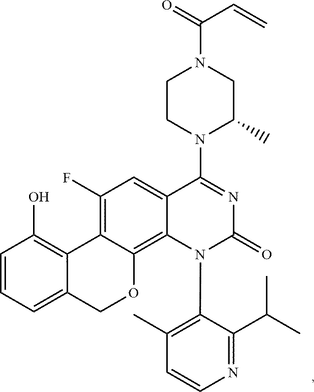 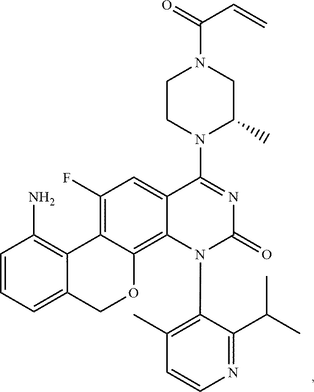 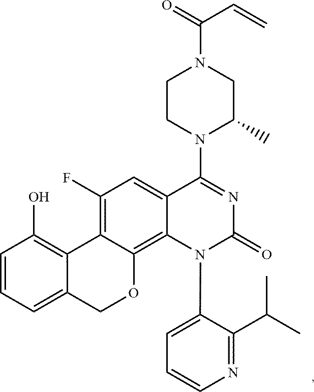 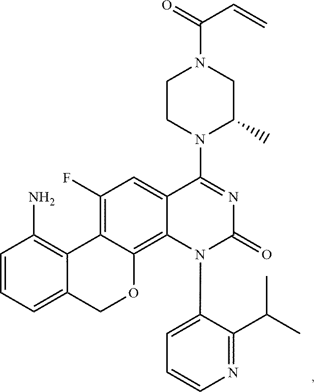 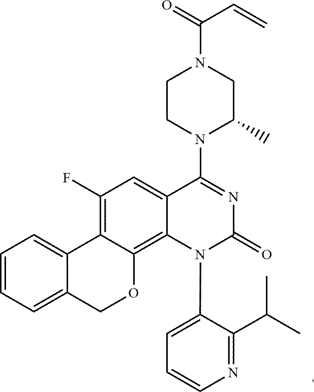 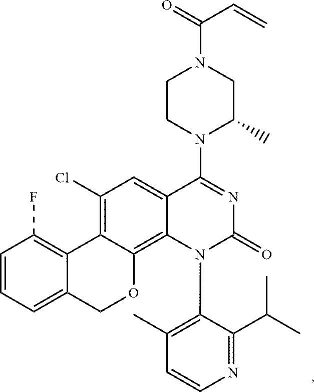 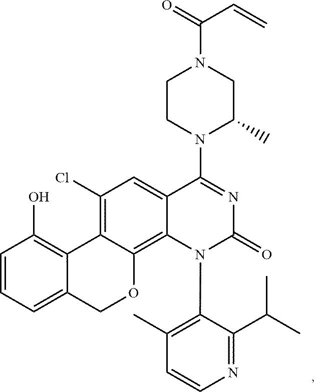 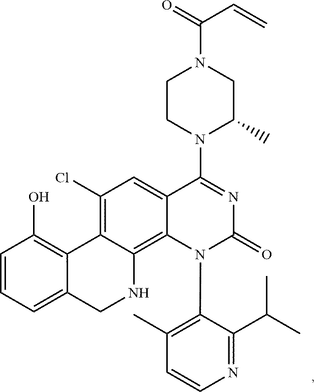 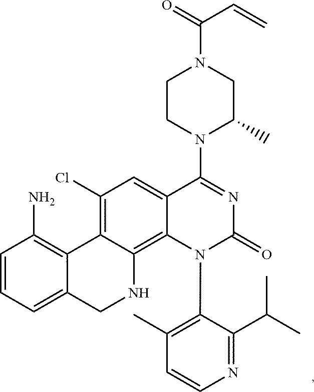 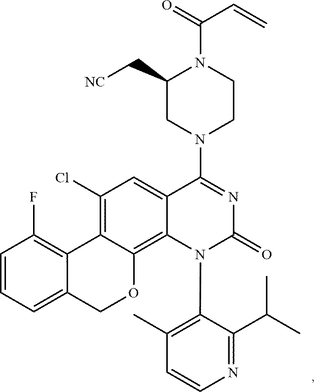 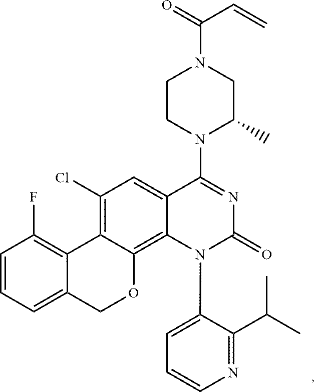 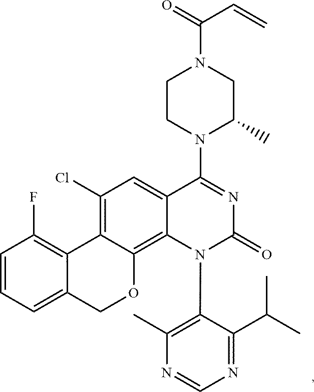 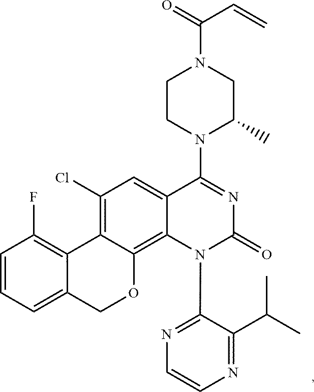 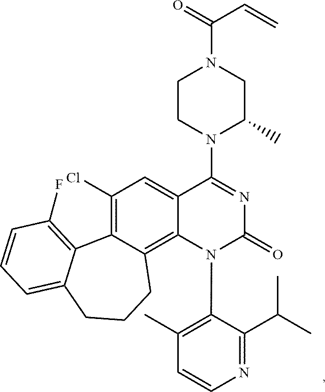 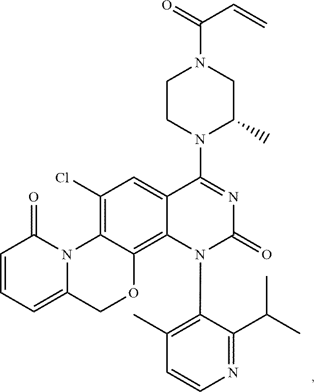 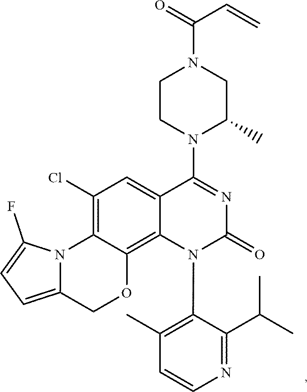 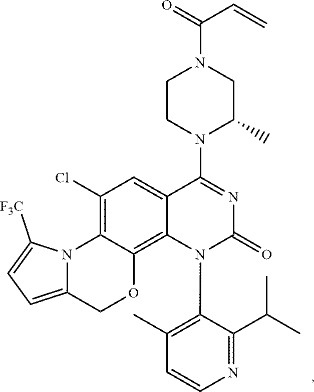 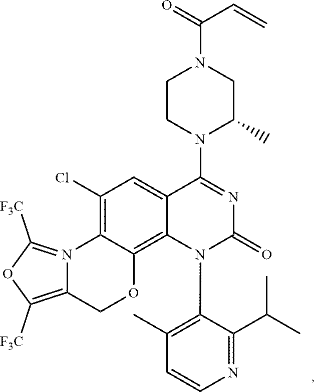 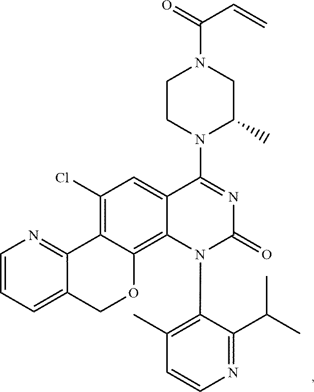 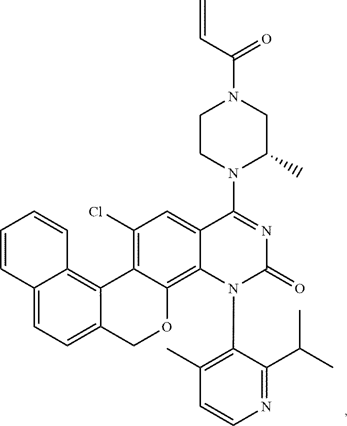 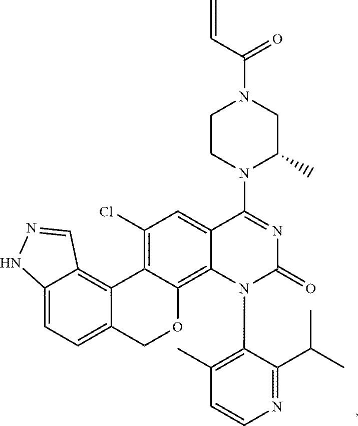 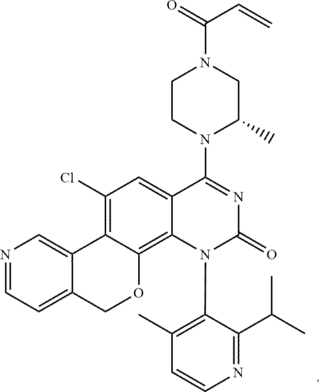 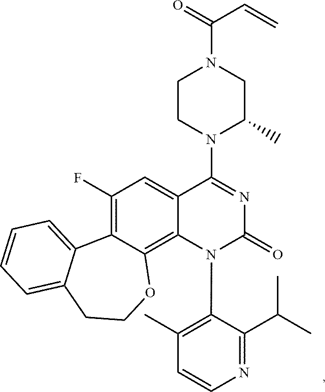 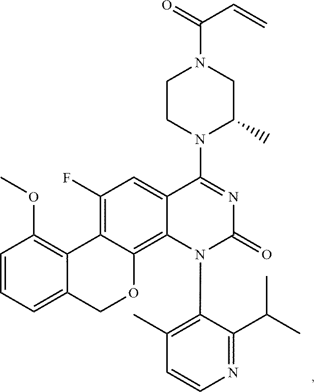 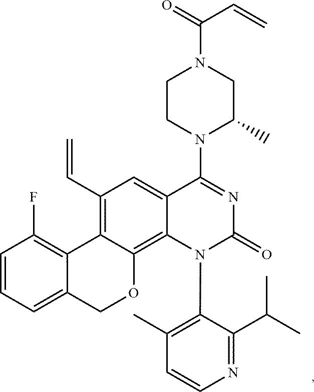 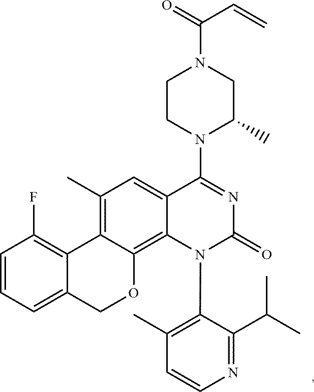 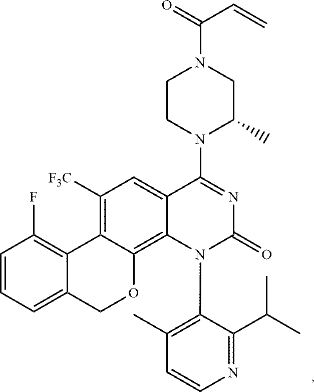 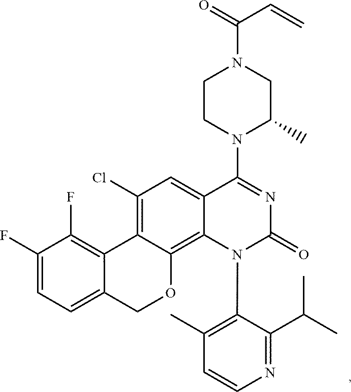 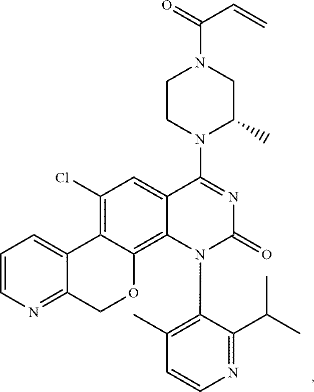 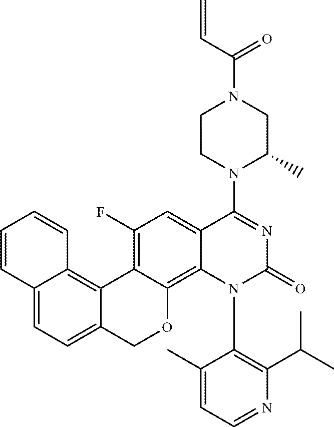 [SMILES: C=CC(=O)N1CCN(c2nc(=O)n(-c3c(C)ccnc3C(C)C)c3c4c(c(F)cc23)-c2c(ccc3ccccc23)CO4)[C@@H](C)C1] 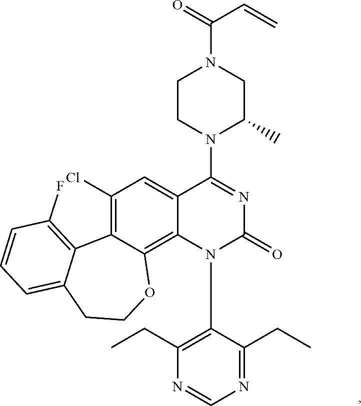 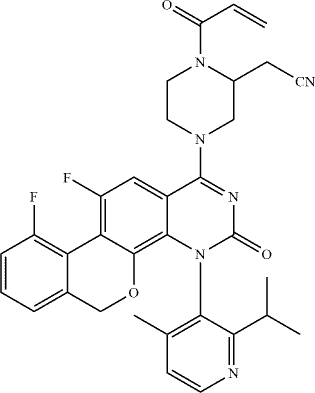 [SMILES: C=CC(=O)N1CCN(c2nc(=O)n(-c3c(C)ccnc3C(C)C)c3c4c(c(F)cc23)-c2c(F)cccc2CO4)CC1CC#N] 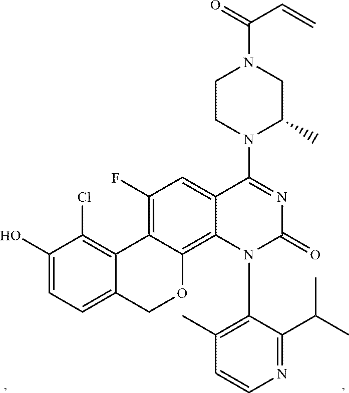 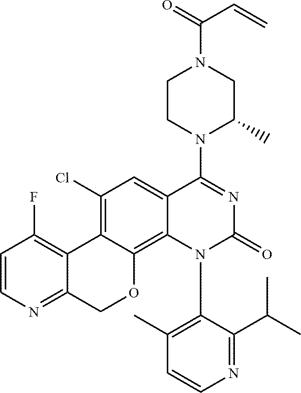 [SMILES: C=CC(=O)N1CCN(c2nc(=O)n(-c3c(C)ccnc3C(C)C)c3c4c(c(Cl)cc23)-c2c(F)ccnc2CO4)[C@@H](C)C1] 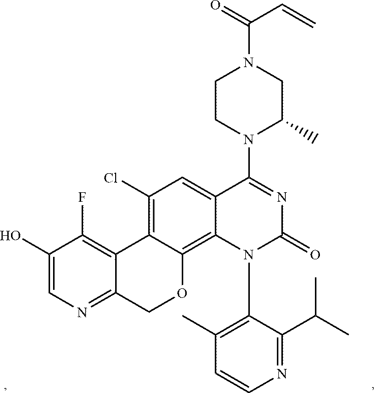 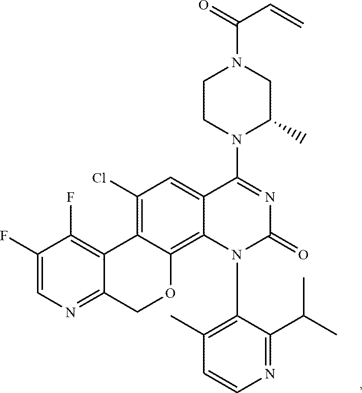 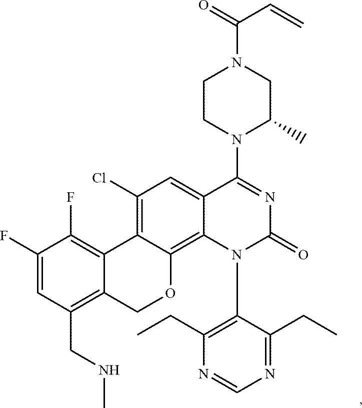 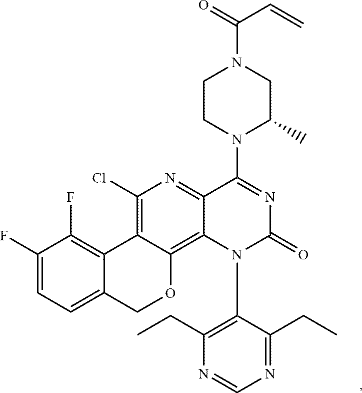 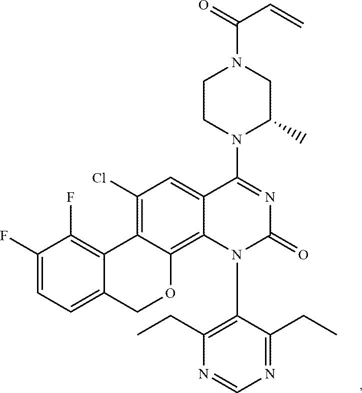 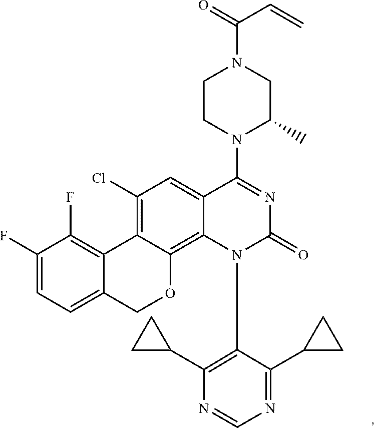 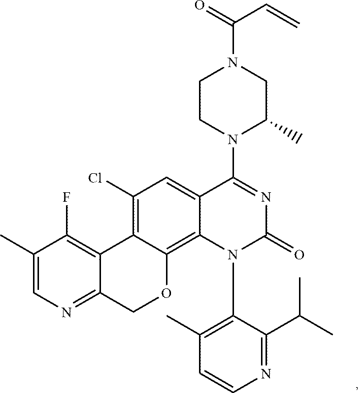 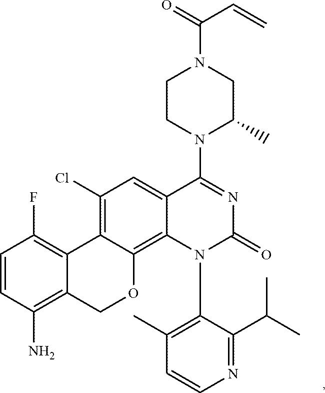 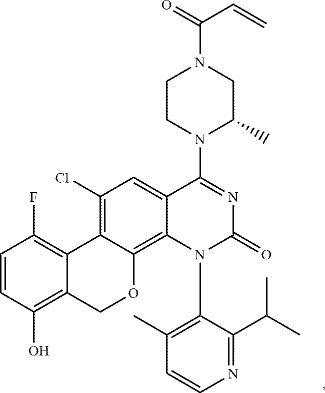 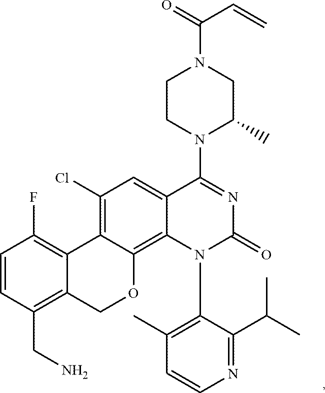 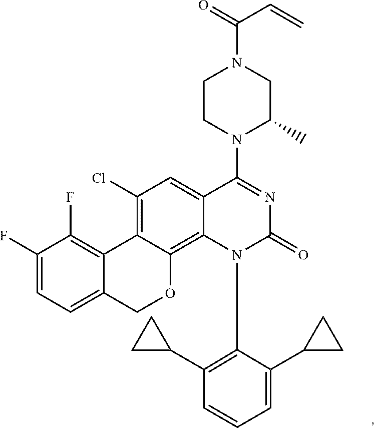 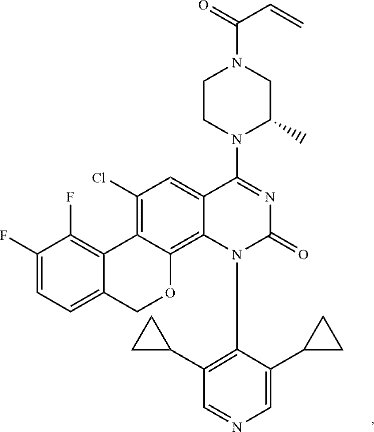 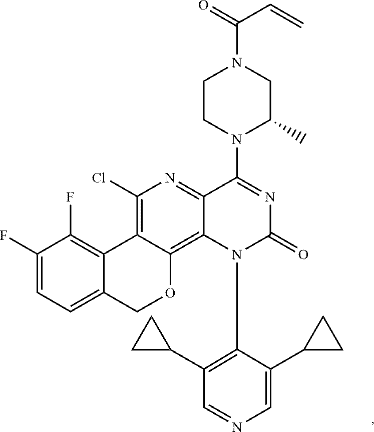 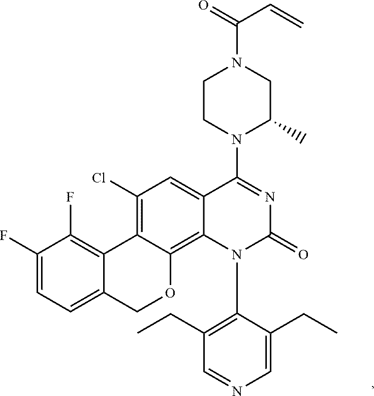 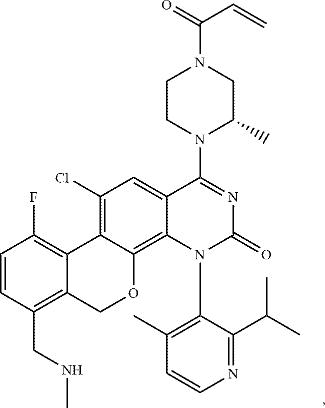 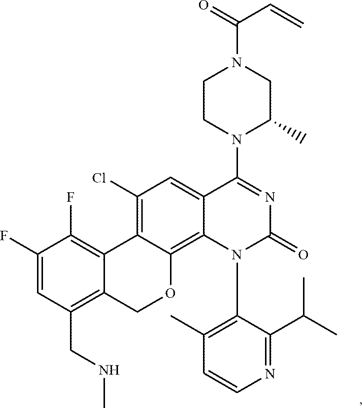 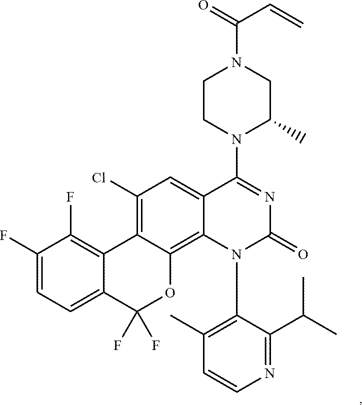 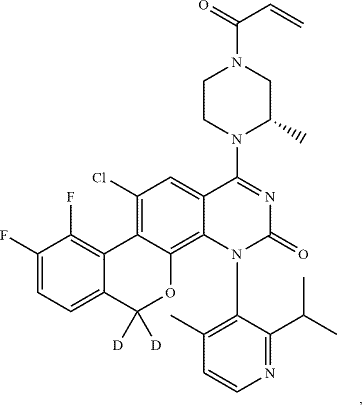 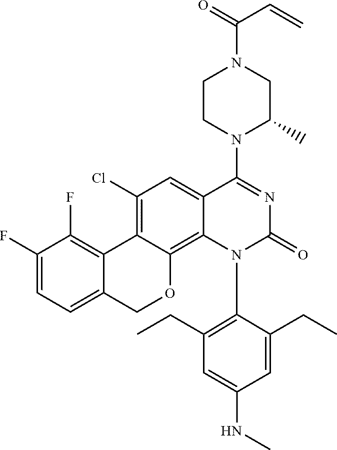 [SMILES: C=CC(=O)N1CCN(c2nc(=O)n(-c3c(CC)cc(NC)cc3CC)c3c4c(c(Cl)cc23)-c2c(ccc(F)c2F)CO4)[C@@H](C)C1] 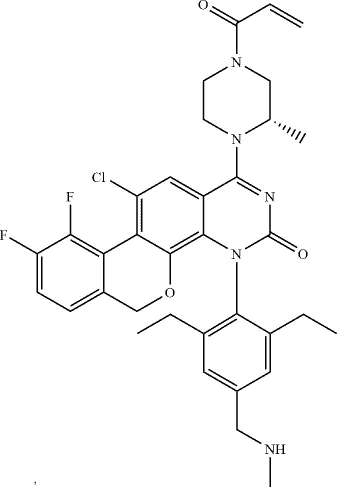 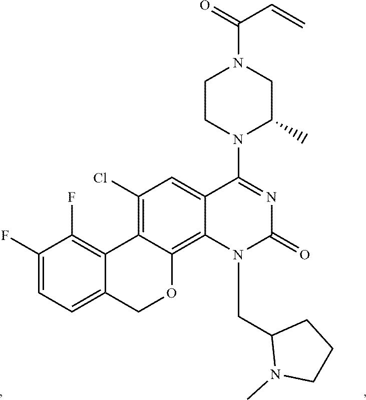 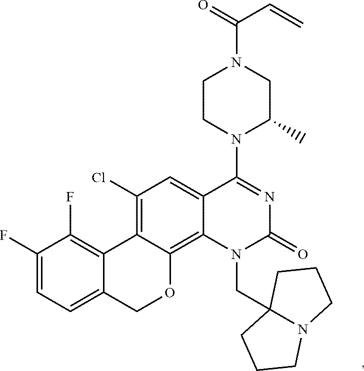 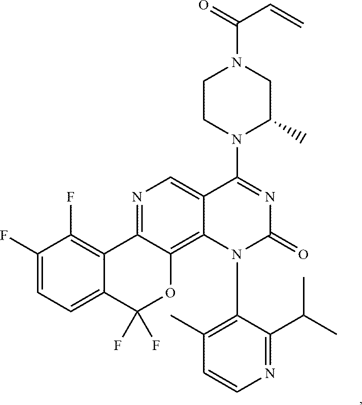 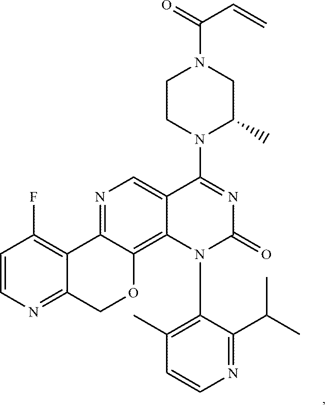 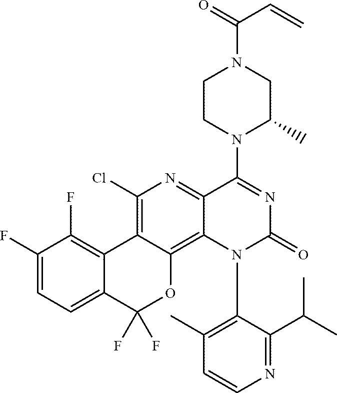 [SMILES: C=CC(=O)N1CCN(c2nc(=O)n(-c3c(C)ccnc3C(C)C)c3c4c(c(Cl)nc23)-c2c(ccc(F)c2F)C(F)(F)O4)[C@@H](C)C1] 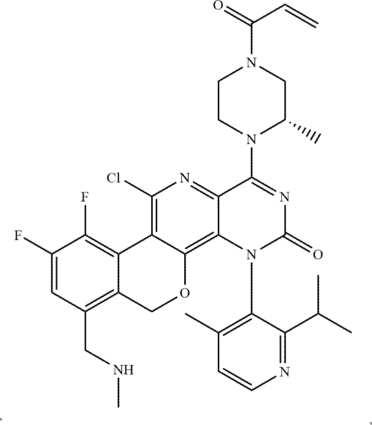 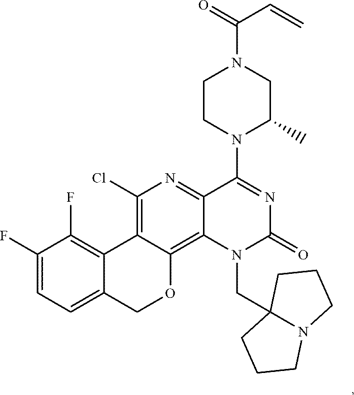 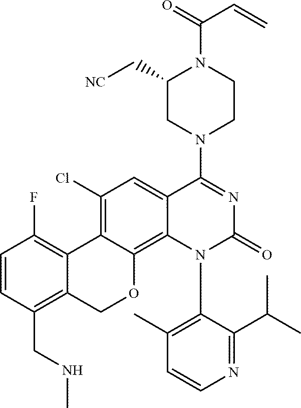 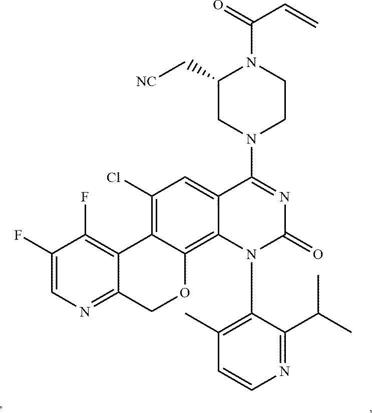 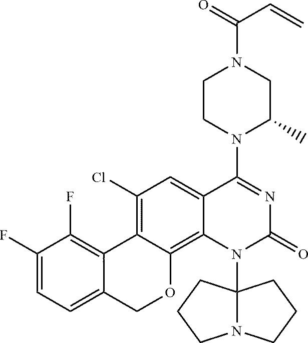 [SMILES: C=CC(=O)N1CCN(c2nc(=O)n(C34CCCN3CCC4)c3c4c(c(Cl)cc23)-c2c(ccc(F)c2F)CO4)[C@@H](C)C1] 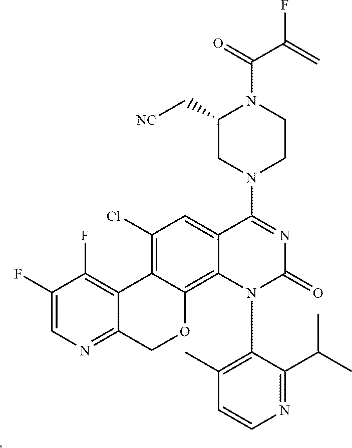 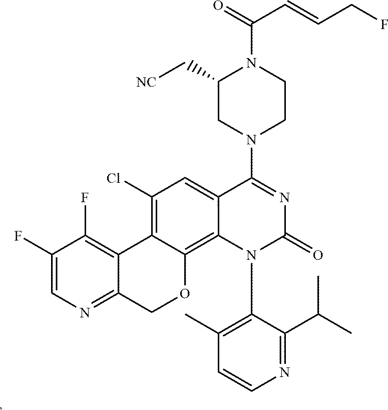 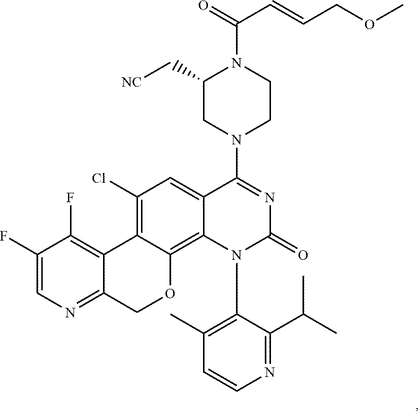 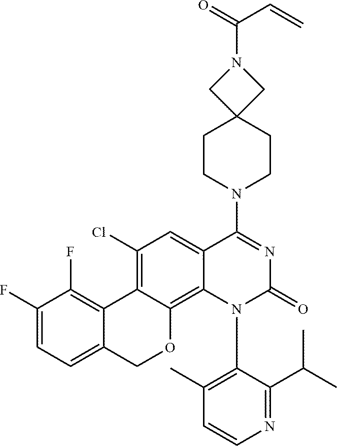 [SMILES: C=CC(=O)N1CC2(CCN(c3nc(=O)n(-c4c(C)ccnc4C(C)C)c4c5c(c(Cl)cc34)-c3c(ccc(F)c3F)CO5)CC2)C1] 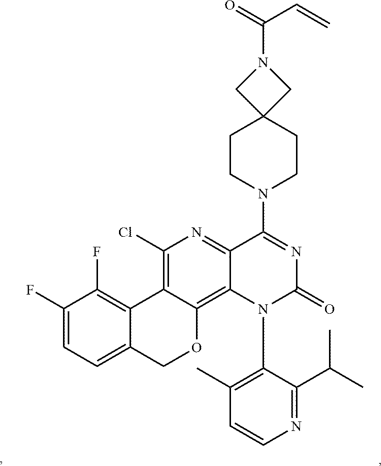 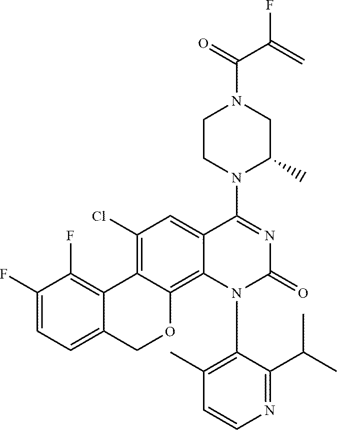 [SMILES: C=C(F)C(=O)N1CCN(c2nc(=O)n(-c3c(C)ccnc3C(C)C)c3c4c(c(Cl)cc23)-c2c(ccc(F)c2F)CO4)[C@@H](C)C1] 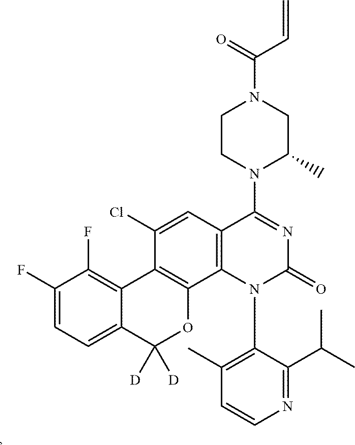 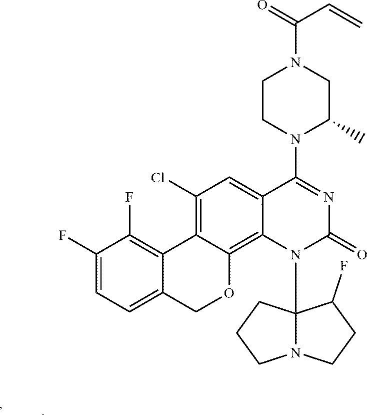 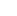 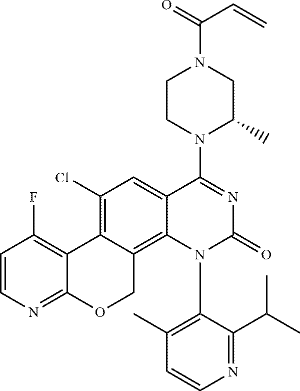 [SMILES: C=CC(=O)N1CCN(c2nc(=O)n(-c3c(C)ccnc3C(C)C)c3c4c(c(Cl)cc23)-c2c(F)ccnc2OC4)[C@@H](C)C1] 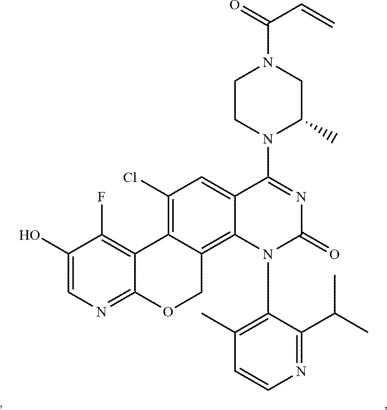 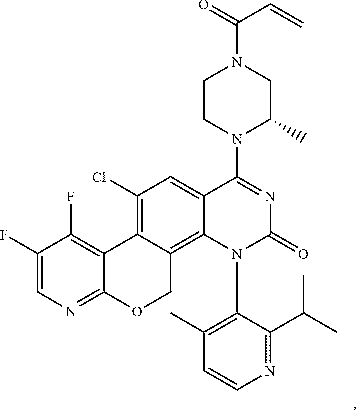 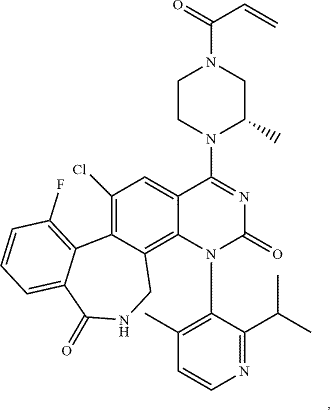 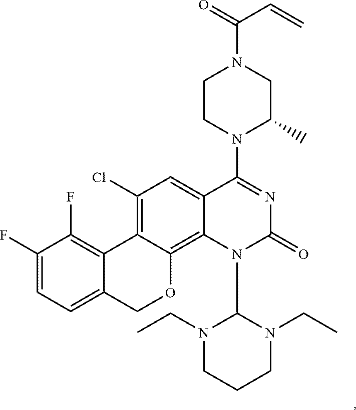 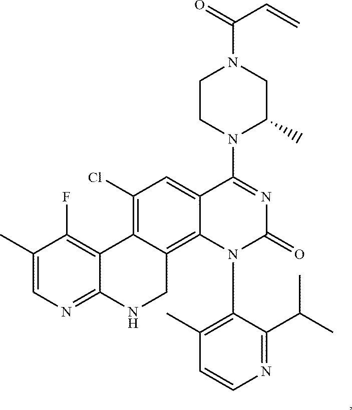 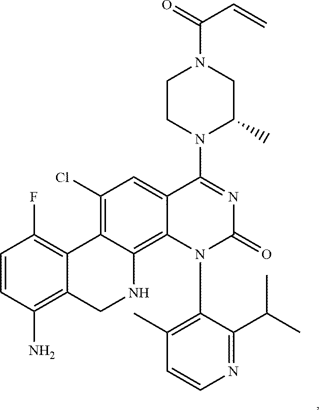 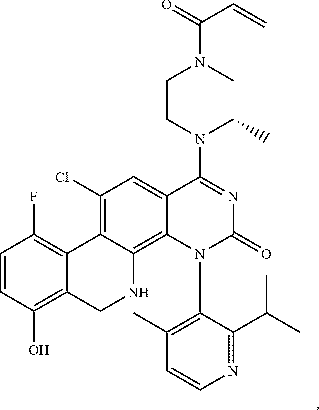 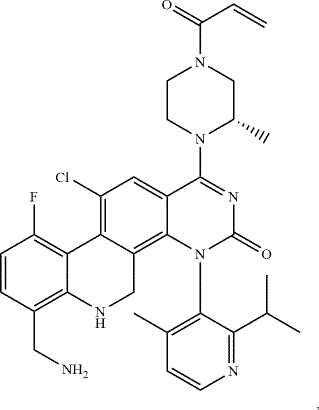 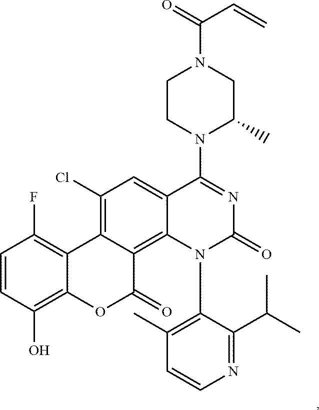 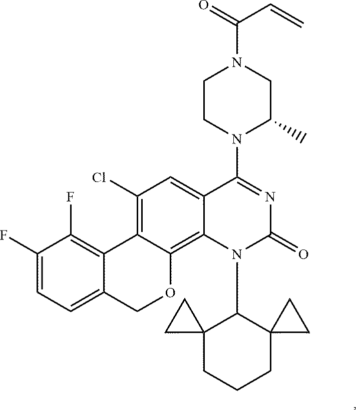 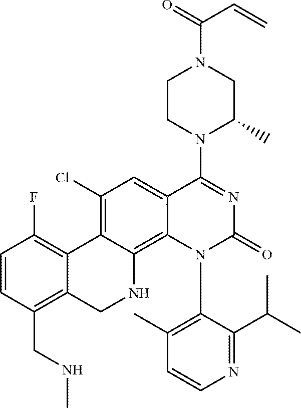 [SMILES: C=CC(=O)N1CCN(c2nc(=O)n(-c3c(C)ccnc3C(C)C)c3c4c(c(Cl)cc23)-c2c(F)ccc(CNC)c2CN4)[C@@H](C)C1] 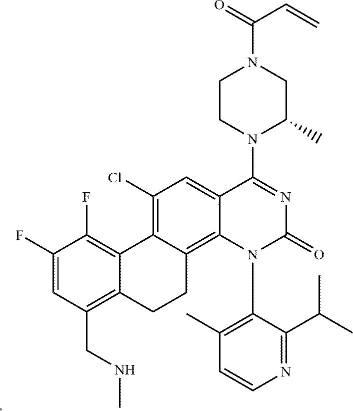 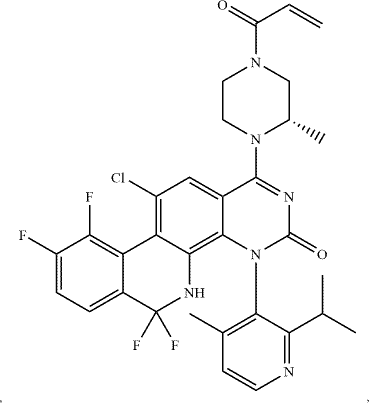 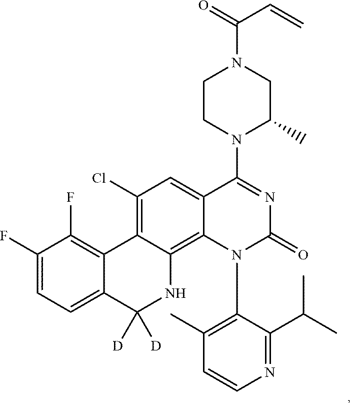 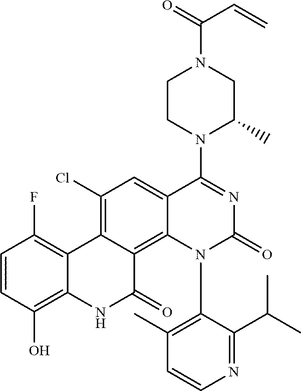 [SMILES: C=CC(=O)N1CCN(c2nc(=O)n(-c3c(C)ccnc3C(C)C)c3c2cc(Cl)c2c4c(F)ccc(O)c4[nH]c(=O)c23)[C@@H](C)C1] 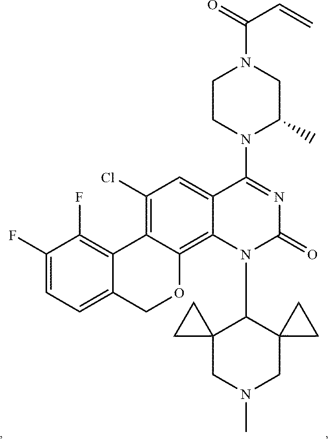 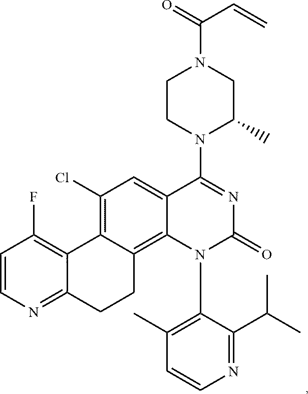 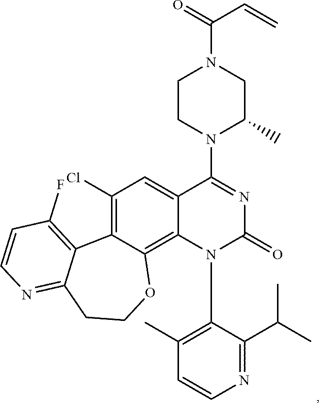 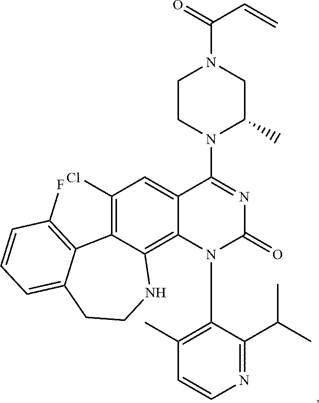 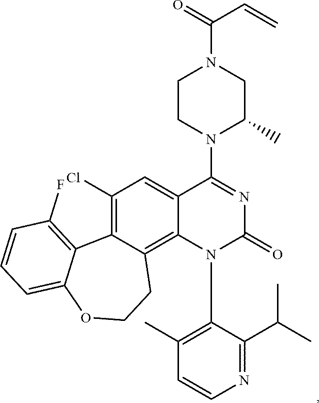 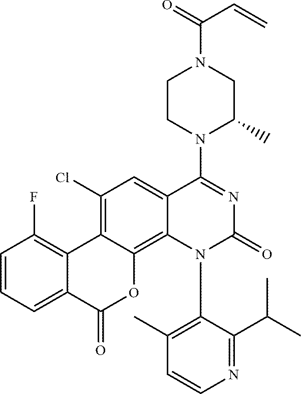 [SMILES: C=CC(=O)N1CCN(c2nc(=O)n(-c3c(C)ccnc3C(C)C)c3c2cc(Cl)c2c3oc(=O)c3cccc(F)c32)[C@@H](C)C1] 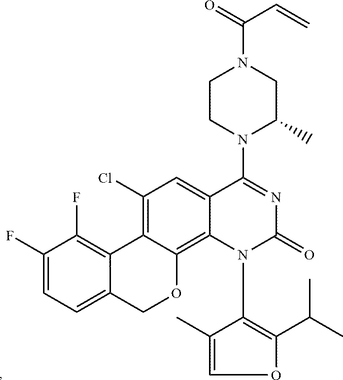 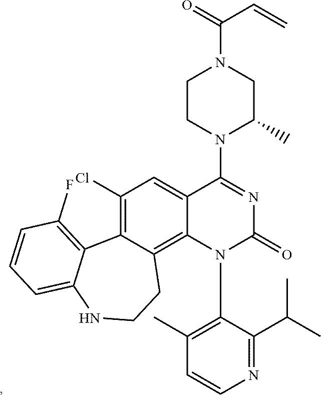 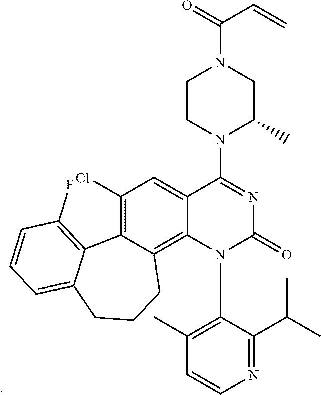 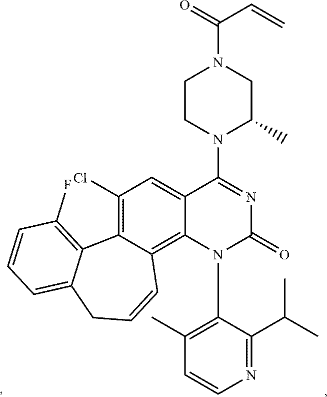 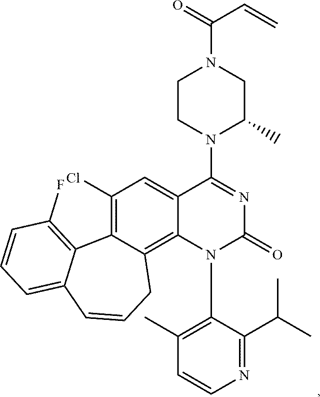 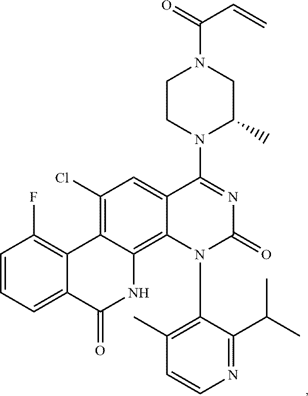 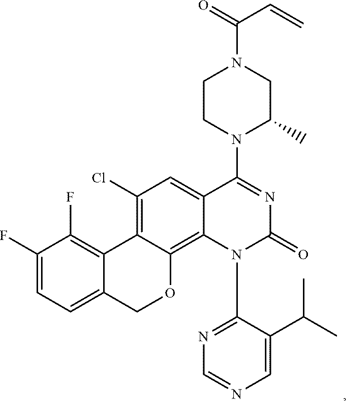 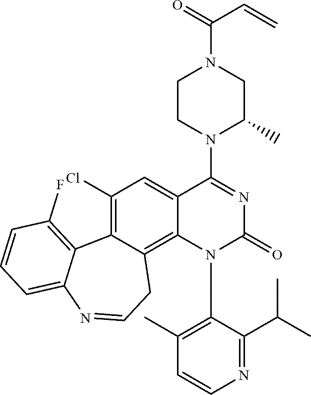 [SMILES: C=CC(=O)N1CCN(c2nc(=O)n(-c3c(C)ccnc3C(C)C)c3c4c(c(Cl)cc23)-c2c(F)cccc2N=CC4)[C@@H](C)C1] 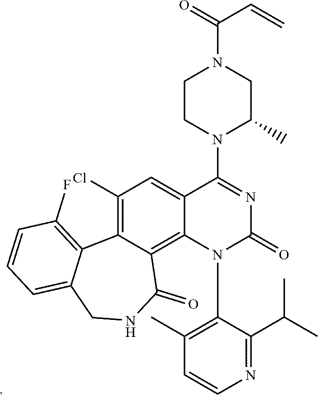 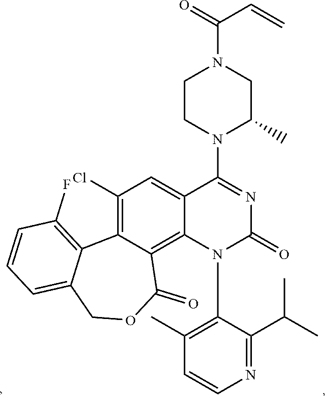 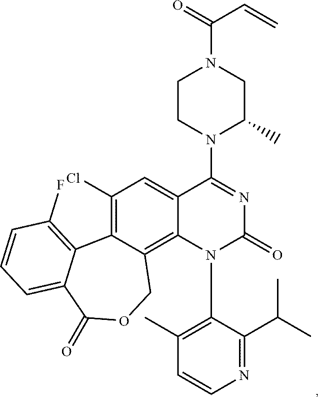 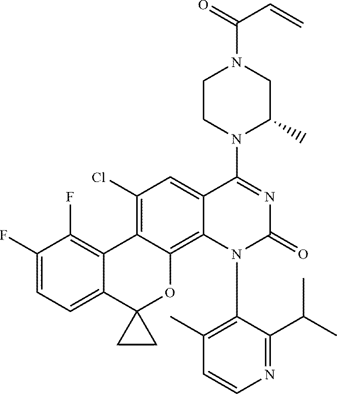 [SMILES: C=CC(=O)N1CCN(c2nc(=O)n(-c3c(C)ccnc3C(C)C)c3c4c(c(Cl)cc23)-c2c(ccc(F)c2F)C2(CC2)O4)[C@@H](C)C1] 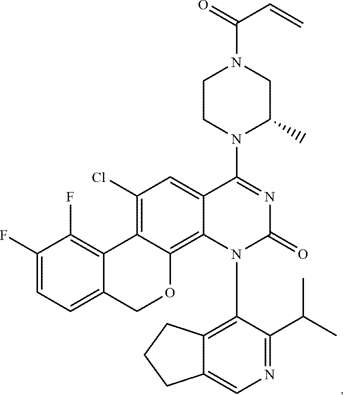 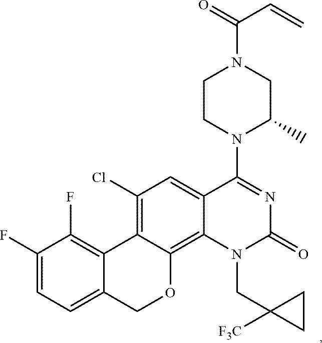 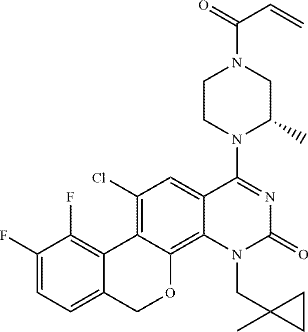 [SMILES: C=CC(=O)N1CCN(c2nc(=O)n(CC3(C)CC3)c3c4c(c(Cl)cc23)-c2c(ccc(F)c2F)CO4)[C@@H](C)C1] 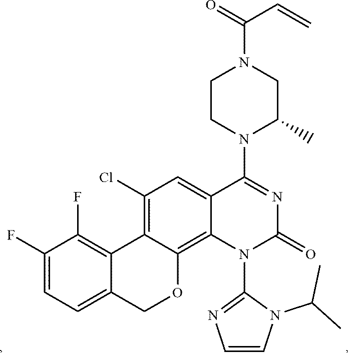 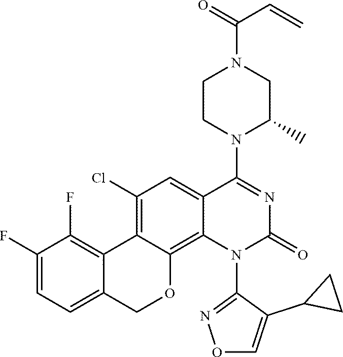 [SMILES: C=CC(=O)N1CCN(c2nc(=O)n(-c3nocc3C3CC3)c3c4c(c(Cl)cc23)-c2c(ccc(F)c2F)CO4)[C@@H](C)C1] 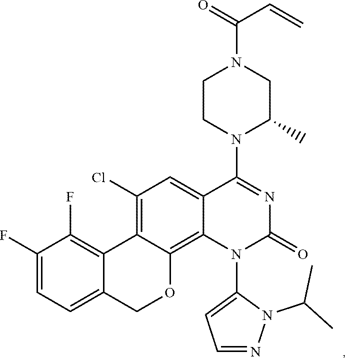 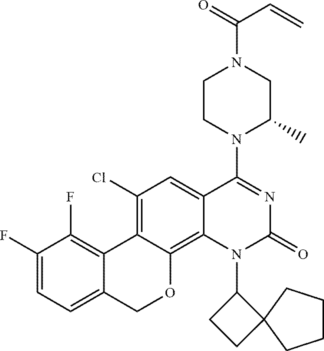 [SMILES: C=CC(=O)N1CCN(c2nc(=O)n(C3CCC34CCCC4)c3c4c(c(Cl)cc23)-c2c(ccc(F)c2F)CO4)[C@@H](C)C1] 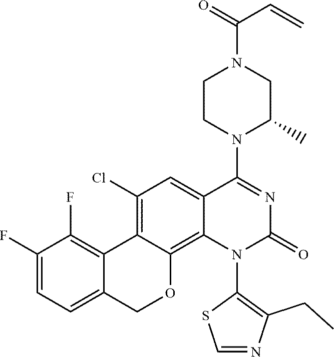 [SMILES: C=CC(=O)N1CCN(c2nc(=O)n(-c3scnc3CC)c3c4c(c(Cl)cc23)-c2c(ccc(F)c2F)CO4)[C@@H](C)C1] 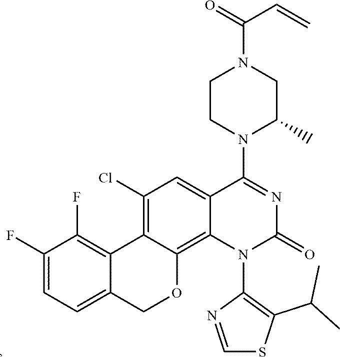 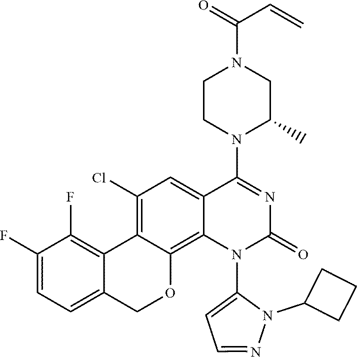 [SMILES: C=CC(=O)N1CCN(c2nc(=O)n(-c3ccnn3C3CCC3)c3c4c(c(Cl)cc23)-c2c(ccc(F)c2F)CO4)[C@@H](C)C1] 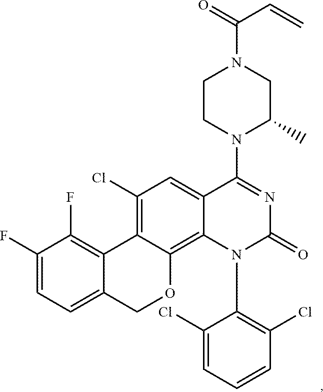 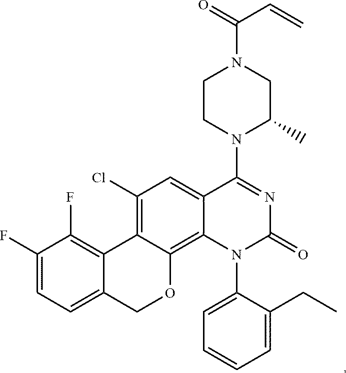 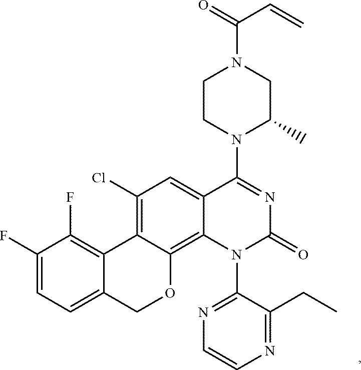 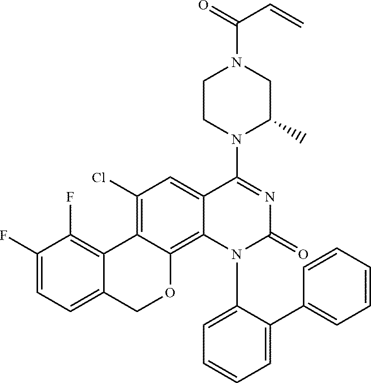 [SMILES: C=CC(=O)N1CCN(c2nc(=O)n(-c3ccccc3-c3ccccc3)c3c4c(c(Cl)cc23)-c2c(ccc(F)c2F)CO4)[C@@H](C)C1] 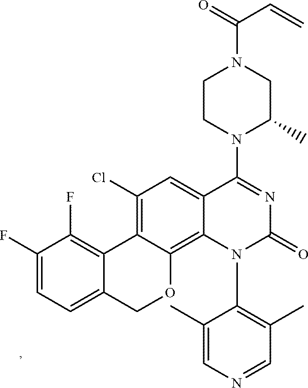 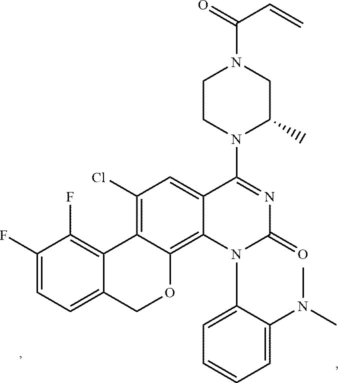 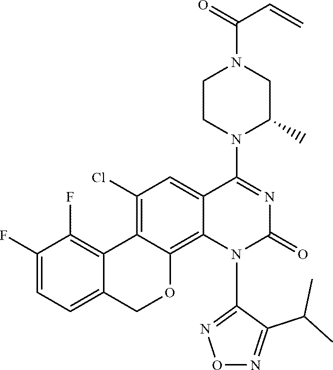 [SMILES: C=CC(=O)N1CCN(c2nc(=O)n(-c3nonc3C(C)C)c3c4c(c(Cl)cc23)-c2c(ccc(F)c2F)CO4)[C@@H](C)C1] 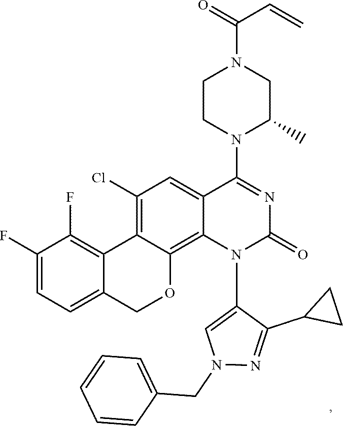 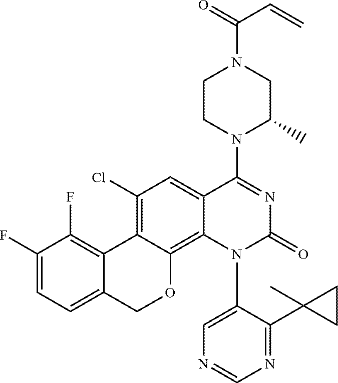 [SMILES: C=CC(=O)N1CCN(c2nc(=O)n(-c3cncnc3C3(C)CC3)c3c4c(c(Cl)cc23)-c2c(ccc(F)c2F)CO4)[C@@H](C)C1] 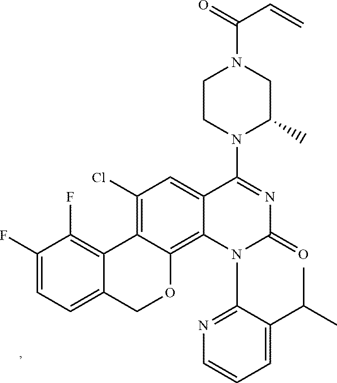 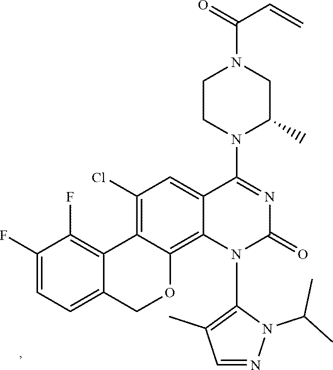 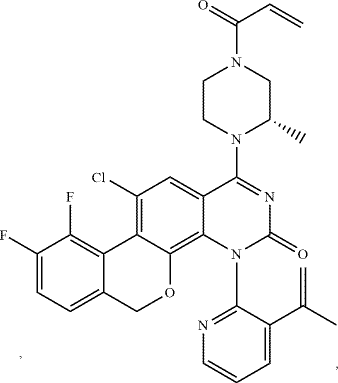 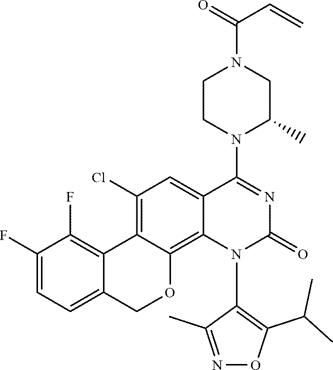 [SMILES: C=CC(=O)N1CCN(c2nc(=O)n(-c3c(C)noc3C(C)C)c3c4c(c(Cl)cc23)-c2c(ccc(F)c2F)CO4)[C@@H](C)C1] 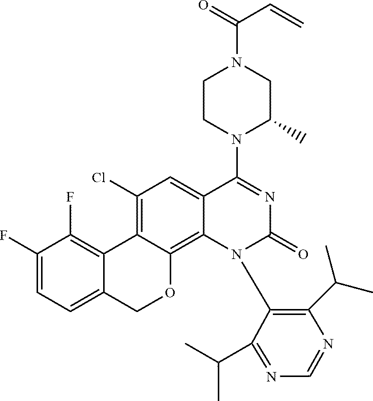 [SMILES: C=CC(=O)N1CCN(c2nc(=O)n(-c3c(C(C)C)ncnc3C(C)C)c3c4c(c(Cl)cc23)-c2c(ccc(F)c2F)CO4)[C@@H](C)C1] 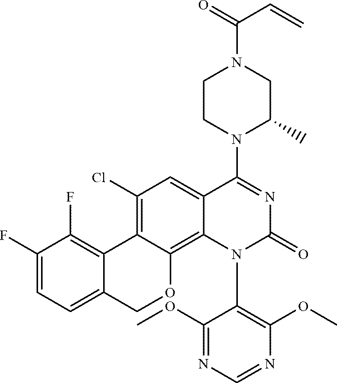 [SMILES: C=CC(=O)N1CCN(c2nc(=O)n(-c3c(OC)ncnc3OC)c3c4c(c(Cl)cc23)-c2c(ccc(F)c2F)CO4)[C@@H](C)C1] 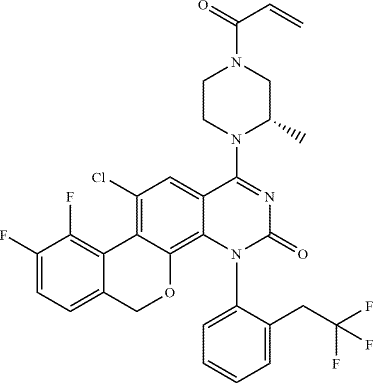 [SMILES: C=CC(=O)N1CCN(c2nc(=O)n(-c3ccccc3CC(F)(F)F)c3c4c(c(Cl)cc23)-c2c(ccc(F)c2F)CO4)[C@@H](C)C1] 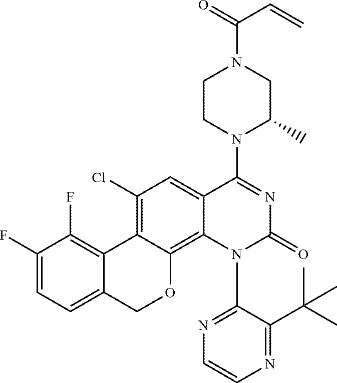 [SMILES: C=CC(=O)N1CCN(c2nc(=O)n(-c3nccnc3C(C)(C)C)c3c4c(c(Cl)cc23)-c2c(ccc(F)c2F)CO4)[C@@H](C)C1] 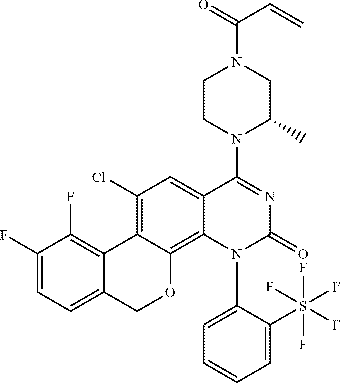 [SMILES: C=CC(=O)N1CCN(c2nc(=O)n(-c3ccccc3S(F)(F)(F)(F)F)c3c4c(c(Cl)cc23)-c2c(ccc(F)c2F)CO4)[C@@H](C)C1] 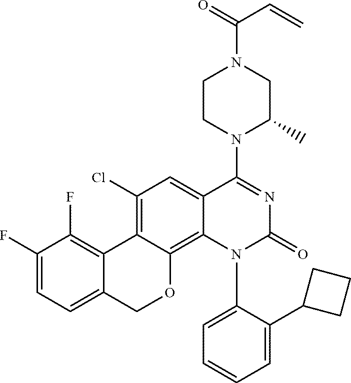 [SMILES: C=CC(=O)N1CCN(c2nc(=O)n(-c3ccccc3C3CCC3)c3c4c(c(Cl)cc23)-c2c(ccc(F)c2F)CO4)[C@@H](C)C1] 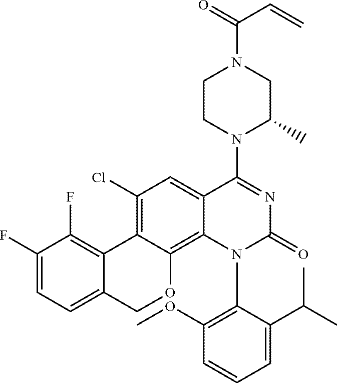 [SMILES: C=CC(=O)N1CCN(c2nc(=O)n(-c3c(OC)cccc3C(C)C)c3c4c(c(Cl)cc23)-c2c(ccc(F)c2F)CO4)[C@@H](C)C1] 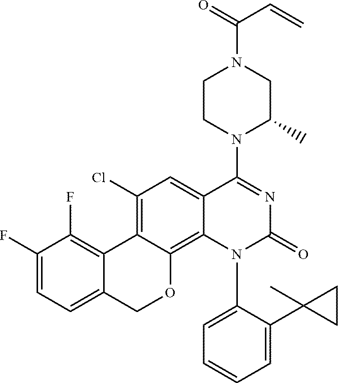 [SMILES: C=CC(=O)N1CCN(c2nc(=O)n(-c3ccccc3C3(C)CC3)c3c4c(c(Cl)cc23)-c2c(ccc(F)c2F)CO4)[C@@H](C)C1] 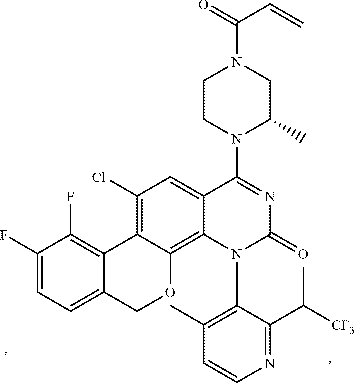 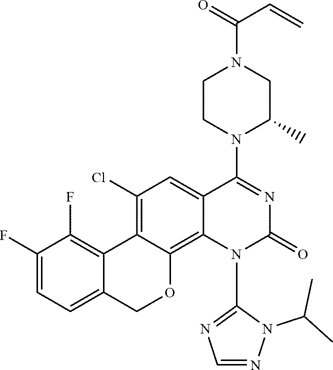 [SMILES: C=CC(=O)N1CCN(c2nc(=O)n(-c3ncnn3C(C)C)c3c4c(c(Cl)cc23)-c2c(ccc(F)c2F)CO4)[C@@H](C)C1] 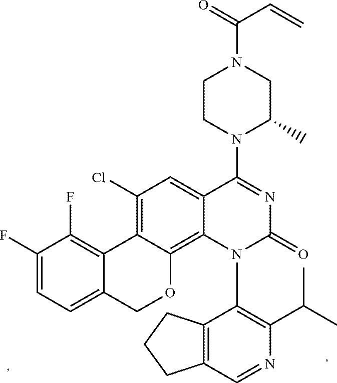 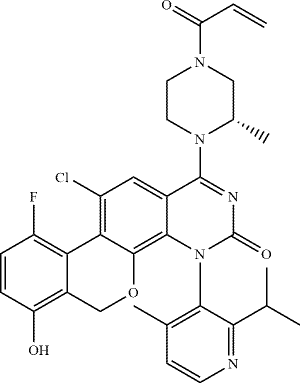 [SMILES: C=CC(=O)N1CCN(c2nc(=O)n(-c3c(C)ccnc3C(C)C)c3c4c(c(Cl)cc23)-c2c(F)ccc(O)c2CO4)[C@@H](C)C1] 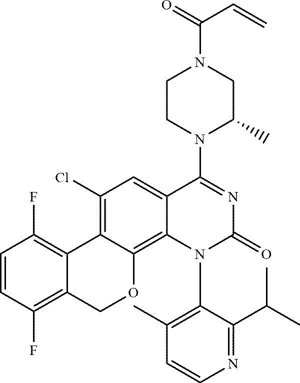 [SMILES: C=CC(=O)N1CCN(c2nc(=O)n(-c3c(C)ccnc3C(C)C)c3c4c(c(Cl)cc23)-c2c(F)ccc(F)c2CO4)[C@@H](C)C1] 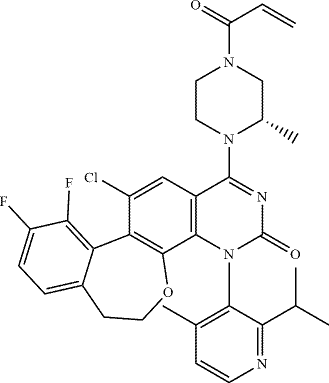 [SMILES: C=CC(=O)N1CCN(c2nc(=O)n(-c3c(C)ccnc3C(C)C)c3c4c(c(Cl)cc23)-c2c(ccc(F)c2F)CCO4)[C@@H](C)C1] 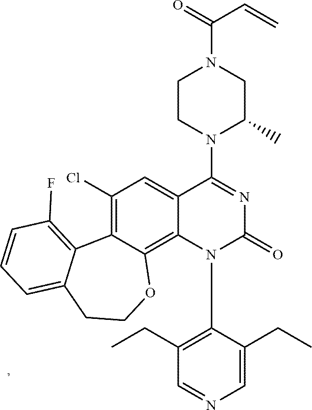 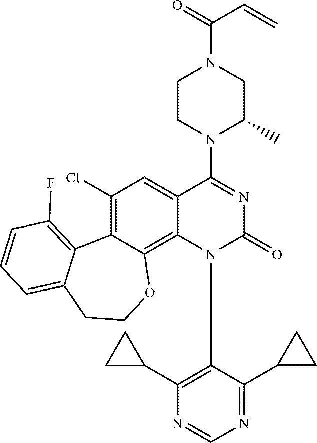 [SMILES: C=CC(=O)N1CCN(c2nc(=O)n(-c3c(C4CC4)ncnc3C3CC3)c3c4c(c(Cl)cc23)-c2c(F)cccc2CCO4)[C@@H](C)C1]
and 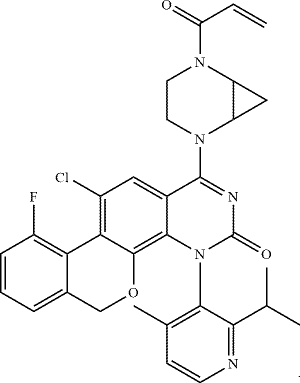
.

18-20. (canceled)

21. A pharmaceutical composition, wherein the pharmaceutical composition contains a therapeutically effective amount of the compound of claim 1 and at least one pharmaceutically acceptable excipient.

22. (canceled)

23. A method of treating and/or preventing a disease, comprising administering a therapeutically effective amount of the compound of claim 1 or a pharmaceutical composition containing a therapeutically effective amount of the compound of claim 1 and at least one pharmaceutically acceptable excipient to a subject.

24. A method for preparing a compound of Formula (I) of claim 1, wherein the method comprises following steps: 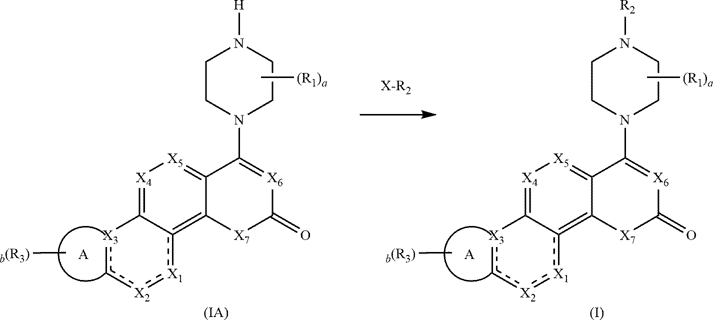
acylating the compound of Formula (IA) with a compound of X-R₂ under basic conditions, to obtain the compound of Formula (I), its tautomer, deuterated compound or pharmaceutically acceptable salt, wherein X is halogen; R₁-R₃, X₁-X₇, a and b are as defined in claim 1.
